(19) United States
(12) Patent Application Publication
(10) Pub. No.: US 2019/0201548 A1
(43) Pub. Date: Jul. 4, 2019

(54) GENE EDITING AND TARGETED TRANSCRIPTIONAL MODULATION FOR ENGINEERING ERYTHROID CELLS

(71) Applicant: RUBIUS THERAPEUTICS, INC., Cambridge, MA (US)

(72) Inventors: Avak Kahvejian, Lexington, MA (US); Jordi Mata-Fink, Somerville, MA (US); Billy Law, Brookline, MA (US)

(21) Appl. No.: 16/235,453

(22) Filed: Dec. 28, 2018

Related U.S. Application Data
(60) Provisional application No. 62/611,604, filed on Dec. 29, 2017.

(51) Int. Cl. A61K 48/00; C12N 5/078; A61P 37/06

(52) U.S. Cl. CPC A61K 48/005; C12N 5/0641; A61K 48/0091; A61K 48/0025; A61P 37/06

(57) ABSTRACT

The disclosure provides, e.g., modified enucleated erythroid cells having increased or decreased levels of particular endogenous proteins. For example, CD47-negative enucleated erythroid cells may be used to induce tolerance to an exogenous antigen.

### RELATED APPLICATIONS

This application claims priority to U.S. Ser. No. 62/611,604 filed Dec. 29, 2017, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Dec. 28, 2018, is named R2081-701910_SL.txt and is 67,204 bytes in size.

### BACKGROUND

Erythroid cells such as red blood cells can be engineered to include a wide variety of exogenous therapeutic proteins in order to treat a number of different diseases. Erythroid cells are a complex chassis for delivering therapeutic proteins, due to the extensive array of endogenous proteins in its cell membrane and in the interior. The endogenous proteins of an erythroid cell affect numerous cell properties, including half-life, immunogenicity, and differentiation. Furthermore, many protein products that are encoded by the genome are absent from fully differentiated erythroid cells, e.g., through lack of expression of the endogenous gene. Wild-type erythroid cells therefore only exhibit a small fraction of the functionality encoded in the genome.

There is a need in the art for new methods of fine-tuning the properties of erythroid cells, including modulating half-life, modulating immunogenicity, and providing new functionalities.

### SUMMARY OF THE INVENTION

This disclosure provides, among other things, erythroid cells (e.g., red blood cells) that have increased or decreased levels of an endogenous protein. For instance, an erythroid cell can have increased levels of a therapeutically useful endogenous protein, in order to administer the cell and therapeutic protein to a subject. As another example, an erythroid cell can have decreased levels of an endogenous immunogenic protein in order to reduce immunogenicity and increase the half-life of the cells in the subject. This regulation can be accomplished, e.g., by exploiting gene editing technology, as described below.

Levels of an endogenous protein can be upregulated or downregulated in several ways as provided herein. For example, Cas9, a TALEN, or a zinc finger protein can be introduced into an erythroid cell which is in the early stages of differentiation and still has a nucleus. The endogenous gene of interest is then edited, e.g., by changing its coding sequence or by increasing or decreasing the strength of its promoter. The cell is then differentiated until the nucleus is lost. At that point, the edited DNA is no longer present, but the impact on protein levels can persist.

As an alternative approach provided herein, levels of an endogenous protein can be changed by upregulating or downregulating transcription in an erythroid cell, without necessarily editing the genomic DNA encoding the protein. Specifically, a nuclease dead Cas9, TAL effector protein, or zinc finger protein can be fused to a transcriptional activator or repressor, creating a targeted transcriptional activator or repressor. The targeted transcriptional activator or repressor can be introduced into a nucleated erythroid cell in early stages of differentiation. The targeted transcriptional activator or repressor then binds to the promoter of an endogenous gene of interest to upregulate or downregulate its expression. The cell is then differentiated until the nucleus is lost. At that point, even if transcription is no longer occurring, the impact on protein levels can persist.

The disclosure provides, in certain aspects, enucleated erythroid cells lacking an endogenous protein or RNA, or having the endogenous protein or RNA present at a level less than about 50%, 40%, 30%, 35%, 20%, 15%, 10%, 9%, 8%, 7%, 6%, 5%, 4%, 3%, 2%, or 1% compared to an unmodified otherwise similar erythroid cell.

The disclosure also provides, in some aspects, an erythroid cell lacking an endogenous protein chosen from CD47, CD58, PLSCR1 (Scramblase), or Bim, or having the endogenous protein present at a level less than about 50%, 40%, 30%, 35%, 20%, 15%, 10%, 9%, 8%, 7%, 6%, 5%, 4%, 3%, 2%, or 1% compared to an unmodified otherwise similar erythroid cell.

The disclosure also provides, in some aspects, enucleated erythroid cell lacking an endogenous protein or RNA, or having the endogenous protein or RNA present at a level less than about 50%, 40%, 30%, 35%, 20%, 15%, 10%, 9%, 8%, 7%, 6%, 5%, 4%, 3%, 2%, or 1% compared to an unmodified otherwise similar erythroid cell, wherein one or more of:
a) the endogenous protein or RNA is present at a level of between 1% and 50% compared to an otherwise similar erythroid cell, e.g., between 1-2%, 2-5%, 5-10%, 10-20%, or 20-50%;
b) the endogenous protein or RNA is expressed by a gene of Table 1 or Table 2;
c) the enucleated erythroid cell comprises an exogenous site-specific DNA binding protein (e.g., a site-specific nuclease), wherein optionally the exogenous site-specific DNA binding protein is selected from a CRISPR polypeptide, e.g., Cas9 polypeptide (e.g., an enzymatically inactive Cas9 polypeptide), ZF polypeptide (e.g., ZFN), TALE polypeptide (e.g., TALEN),
d) the enucleated erythroid cell comprises an adeno-associated virus (AAV) vector;
e) the enucleated erythroid cell does not contain a detectable amount of one or more of: microRNA, siRNA, or shRNA;
f) the enucleated erythroid cell does not contain a detectable amount of active Dicer, RISC, or Ago2;
g) the endogenous protein is other than, or the RNA does not encode, a transcription factor, a growth factor, or a growth factor receptor, including but not limited to, e.g., GATA1, GATA2, CMyc, hTERT, p53, EPO, SCF, insulin, EPO-R, SCF-R, transferrin-R, insulin-R;
h) the enucleated erythroid cell is not from a subject having a hemoglobinopathy, e.g., sickle cell disease or sickle cell trait; or a thalassemia (e.g., alpha thalassemia or beta thalassemia); or
i) the enucleated erythroid cell has wild-type levels and/or sequence of all hemoglobins.

The disclosure also provides, in some aspects, an erythroid cell, e.g., an enucleated erythroid cell, comprising an exogenous site-specific DNA binding protein (e.g., a site-specific nuclease), e.g., at an amount of at least 100, 200, 500, 1,000, 2,000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 9000,000, or 1,000,000 copies per cell, or in an amount of 100-200, 200-500, 500-1,000, 1,000-2,000, 2,000-5,000, 5,000-10,000, 10,000-20,000, 20,000-30,000, 30,000-40,000, 40,000-50,000, 50,000-100,000, 100,000-200,000, 200,000-500,000, or 500,000-1,000,000 copies per cell.

The disclosure also provides, in some aspects, an erythroid cell, e.g., an enucleated erythroid cell, comprising a guide nucleic acid, e.g., a guide RNA, e.g., a single guide RNA (sgRNA).

The disclosure also provides, in certain aspects, a method of making or manufacturing a modified erythroid cell comprising:
a) providing an erythroid precursor cell comprising a first nucleic acid, e.g., endogenous nucleic acid, e.g., endogenous DNA, e.g., chromosomal nucleic acid, e.g., a cell comprising a nucleus;
b) contacting the erythroid precursor cell with an exogenous nucleic acid-binding protein that specifically binds the first nucleic acid and mediates a first sequence-specific alteration in gene expression, e.g., transcription (e.g., increase in transcription or decrease in transcription), or a second nucleic acid encoding said exogenous nucleic acid-binding protein, under conditions that allow for sequence-specific alteration in transcription, to provide a modified erythroid cell, thereby manufacturing a modified erythroid cell. In some embodiments, specific binding of the exogenous nucleic acid-binding protein to the first nucleic acid is mediated by a nucleic acid, e.g., a gRNA, e.g., a sgRNA. In some embodiments, specific binding of the exogenous nucleic acid-binding protein to the first nucleic acid comprises direct binding of the exogenous nucleic acid-binding protein to the first nucleic acid.

The disclosure also provides, in certain aspects, a method of making or manufacturing a modified erythroid cell comprising:
a) providing an erythroid precursor cell comprising a first nucleic acid, e.g., endogenous nucleic acid, e.g., endogenous DNA, e.g., chromosomal nucleic acid, e.g., a cell comprising a nucleus;
b) contacting the erythroid precursor cell with an exogenous nucleic acid-binding protein of less than 1,300 amino acids in length, which protein specifically binds the first nucleic acid and mediates a first sequence-specific alteration in a sequence of the first nucleic acid or in gene expression, e.g., transcription (e.g., increase in transcription or decrease in transcription), or a second nucleic acid encoding said exogenous nucleic acid-binding protein, under conditions that allow for sequence-specific alteration in transcription, to provide a modified erythroid cell, thereby manufacturing a modified erythroid cell.

The present disclosure also provides, in some aspects, a method of making or manufacturing a modified erythroid cell comprising:
a) providing an erythroid precursor cell comprising a first nucleic acid, e.g., endogenous nucleic acid, e.g., endogenous DNA, e.g., chromosomal nucleic acid, e.g., a cell comprising a nucleus;
b) contacting the erythroid precursor cell with an exogenous nucleic acid-binding protein, which protein specifically binds the first nucleic acid and mediates a first sequence-specific alteration in a sequence of the first nucleic acid or in gene expression, e.g., transcription (e.g., increase in transcription or decrease in transcription), or a second nucleic acid (e.g., RNA) encoding said exogenous nucleic acid-binding protein, under conditions that allow for sequence-specific alteration in transcription, to provide a modified erythroid cell,

wherein the contacting comprises performing transfection (e.g., LNP-mediated transfection, e.g., wherein the LNP is in a complex with the second nucleic acid, e.g., RNA) or electroporation;

thereby manufacturing a modified erythroid cell.

The disclosure also provides, in certain aspects, a modified erythroid cell produced by a method herein.

This disclosure also provides, in some aspects, an erythroid cell, e.g., enucleated erythroid cell, lacking an endogenous CD47 or CD47-encoding nucleic acid (e.g., DNA or RNA), or having the endogenous CD47 or CD47-encoding nucleic acid (e.g., DNA or RNA) present at a level less than about 50%, 40%, 30%, 20%, 10%, 5%, 2%, or 1% compared to an unmodified otherwise similar erythroid cell. This disclosure also provides, in some aspects, a method of inducing immune tolerance, reducing immune activation, or treating an autoimmune disease in a subject, comprising administering a cell or population of cells described herein (e.g., an erythroid cell with reduced endogenous CD47) to a subject in need thereof, thereby inducing immune tolerance, reducing immune activation, or treating the autoimmune disease in the subject. The disclosure also provides, in some aspects, a cell or population described herein (e.g., having reduced endogenous CD47) for use in inducing immune tolerance, reducing immune activation, or treating an autoimmune disease in a subject.

This disclosure also provides, in some aspects, an erythroid cell, e.g., enucleated erythroid cell, lacking an endogenous CD58 or CD58-encoding nucleic acid (e.g., DNA or RNA), or having the endogenous CD58 or CD58-encoding nucleic acid (e.g., DNA or RNA) present at a level less than about 50%, 40%, 30%, 20%, 10%, 5%, 2%, or 1% compared to an unmodified otherwise similar erythroid cell.

This disclosure also provides, in some aspects, an erythroid cell, e.g., enucleated erythroid cell, comprising the endogenous CD58 or CD58-encoding nucleic acid (e.g., DNA or RNA) at a level greater than about 10%, 20%, 30%, 40%, 50%, 60%, 80%, 2-fold, 3-fold, 4-fold, 5-fold, or 10-fold compared to an unmodified otherwise similar erythroid cell. This disclosure also provides, in some aspects, a method of inducing immune tolerance, reducing immune activation, or treating an autoimmune disease in a subject, comprising administering a cell described herein (e.g., an erythroid cell comprising increased endogenous CD58) to a subject in need thereof, thereby inducing immune tolerance, reducing immune activation, or treating the autoimmune disease in the subject.

This disclosure also provides, in some aspects, an erythroid cell, e.g., enucleated erythroid cell, comprising the endogenous CR1 or CR1-encoding nucleic acid (e.g., DNA or RNA) at a level greater than, or increased by, about 10%, 20%, 30%, 40%, 50%, 60%, 80%, 2-fold, 3-fold, 4-fold, 5-fold, or 10-fold compared to an unmodified otherwise similar erythroid cell. This disclosure also provides, in some aspects, a method of treating an autoimmune disease or reducing levels of an autoantibody in a subject, comprising administering a cell described herein (e.g., a cell with increased endogenous CR1) to a subject in need thereof, thereby treating the autoimmune disease or reducing levels of the autoantibody in the subject.

This disclosure also provides, in some aspects, an erythroid cell, e.g., enucleated erythroid cell, lacking an endogenous differentiation factor or nucleic acid (e.g., DNA or RNA) encoding the differentiation factor, or having the differentiation factor or nucleic acid (e.g., DNA or RNA) encoding the differentiation factor present at a level less than about 50%, 40%, 30%, 20%, 10%, 5%, 2%, or 1% compared to an unmodified otherwise similar erythroid cell.

This disclosure also provides, in some aspects, an erythroid cell, e.g., enucleated erythroid cell, comprising an endogenous differentiation factor or nucleic acid (e.g., DNA or RNA) encoding the differentiation factor at a level greater than, or increased by, about 10%, 20%, 30%, 40%, 50%, 60%, 80%, 2-fold, 3-fold, 4-fold, 5-fold, or 10-fold compared to an unmodified otherwise similar erythroid cell.

This disclosure also provides, in some aspects, an erythroid cell, e.g., enucleated erythroid cell, comprising an endogenous protein of Table 1, or nucleic acid (e.g., DNA or RNA) encoding the endogenous protein, at a level greater than, or increased by, about 10%, 20%, 30%, 40%, 50%, 60%, 80%, 2-fold, 3-fold, 4-fold, 5-fold, or 10-fold compared to an unmodified otherwise similar erythroid cell.

In certain aspects, the disclosure also provides an isolated population of cells comprising enucleated erythroid cells comprising increased levels of a protein of Table 1 or decreased levels of a protein of Table 2, e.g., wherein the cells comprise at least 50%, 60%, 70%, 80%, 85%, or 90% enucleated erythroid cells. In certain aspects, the disclosure also provides an isolated population of cells made by a method described herein, e.g., wherein the cells comprise at least 50%, 60%, 70%, 80%, 85%, or 90% enucleated erythroid cells.

In any of the aspects herein, e.g., the compositions and methods above, any of the embodiments below may apply.

In some embodiments, the enucleated erythroid cell is substantially purified. In some embodiments, the enucleated erythroid cell is ex vivo. In some embodiments, the enucleated erythroid cell is a reticulocyte or a mature red blood cell. In some embodiments, the enucleated erythroid cell further comprises an exogenous polypeptide. In some embodiments, the enucleated erythroid cell further comprises a second exogenous polypeptide. In some embodiments, the erythroid cell lacks one or more endogenous proteins. In some embodiments, the endogenous protein present at a level less than about 90%, 80%, 70%, 60%, 50%, 40%, 30%, 20%, 15%, 10%, 9%, 8%, 7%, 6%, 5%, 4%, 3%, 2%, or 1% as compared to an unmodified otherwise similar erythroid cell.

In some embodiments, the endogenous protein is selected from a transcription factor, red blood cell transmembrane protein, a phospholipid transportation enzyme (e.g., a scramblase), or a pro-apoptotic protein.

In some embodiments, the enucleated erythroid cell was produced using an exogenous site-specific DNA binding protein (e.g., a site-specific nuclease), wherein optionally the exogenous site-specific DNA binding protein is selected from a Cas9 polypeptide (e.g., a nuclease dead Cas9 polypeptide), ZF polypeptide (e.g., ZFN), TALE polypeptide (e.g., TALEN), or a viral vector component, e.g., a DNA or RNA viral vector component, e.g., an AAV vector, e.g., a self-complementary adeno-associated virus (scAAV) comprising double-stranded DNA vector component, e.g., wherein the viral vector component comprises an exogenous nucleic acid sequence, e.g., an exogenous nucleic acid sequence flanked by inverted terminal repeats (ITRs). In some embodiments, the Cas9 polypeptide comprises a nuclease-dead Cas9 polypeptide or a Cas9 polypeptide having nuclease activity. In some embodiments, the exogenous site-specific DNA binding protein is selected from a Cas9 polypeptide (e.g., a nuclease dead Cas9 polypeptide), ZF polypeptide (e.g., ZFN), or TALE polypeptide (e.g., TALEN). In some embodiments, the site-specific DNA binding protein further requires a nuclear localization sequence (NLS), e.g., at the N-terminus of the site-specific DNA binding protein.

In some embodiments, enucleated erythroid cell was produced using a nucleic acid inhibitor, e.g., siRNA or shRNA.

In some embodiments, the enucleated erythroid cell does not contain a detectable amount of an exogenous microRNA, or the enucleated erythroid cells contains less than 1,000, 100, or 10 copies per cell of an exogenous microRNA. In some embodiments, the enucleated erythroid cell does not contain a detectable amount of siRNA or the enucleated erythroid cells contains less than 1,000, 100, or 10 copies per cell of siRNA. In some embodiments, the enucleated erythroid cell does not contain a detectable amount of shRNA or the enucleated erythroid cells contains less than 1,000, 100, or 10 copies per cell of shRNA.

In some embodiments, the Cas9 polypeptide is derived from a microorganism, e.g., a bacterium, e.g. a proteobacterium (e.g., a gammaproteobacterium, a betaproteobacterium, alphaproteobacterium, a deltaproteobacterium, or an epsilonproteobacterium), a firmicute (e.g., a bacillus), or a spirochaete. In some embodiments, the Cas9 polypeptide is derived from a microorganism selected from the group consisting of: a Streptococcus sp. (e.g., Streptococcus pyogenes, Streptococcus thermophilus, Streptococcus pseudoporcinus, Streptococcus mutans, Streptococcus macacae, Streptococcus gallolyliticus, Streptococcus equinus, Streptococcus dysdalactiae, Streptococcus bovis, Streptococcus cmginosus, or Streptococcus agalactiae) a Staphylococcus sp. (e.g., Staphylococcus lugdunensis or Staphylococcus aureus), a Neisseria sp. (e.g., Neisseria meningitides), a Enterococcus sp. (e.g., Enerococcus italicus or Enterococcus faecium), an Acidovorax sp. (e.g., Acidovorax avenae), an Actinobacillus sp. (e.g., Actinobacillus pleuropneumoniae, Actinobacillus succinogenes, or Actinobacillus suis), an Actinomyces sp., a Cycliphilus sp. (e.g., Cycliphilus denitrificans), a Aminomonas sp. (e.g., Aminomonas paucivorans), a Bacillus sp. (Bacillus cereus, Bacillus smithii, or Bacillus thuringiensis), a Bacteroides sp., a Blastopirellula sp. (e.g., Blastopirellula marina), a Bradyrhizobium sp., a Brevibacillus sp. (Brevibacillus latemsporus), a Campylobacter sp. (e.g., Campylobacter coli, Campylobacter jejuni, or Campylobacter lad), a Candidatus sp. (e.g., Candidatus Puniceispirillum sp.), a Clostridium sp. (e.g., Clostridium cellulolyticum or Clostridium perfringens), a Corynebacterium sp. (e.g., Corynebacterium accolens, Corynebacterium diphtheria, or Corynebacterium matruchotii), a Dinoroseobacter sp. (e.g., Dinoroseobacter sliibae), a Eubacterium (e.g., Eubacterium dolichum), Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, a Listeria sp. (e.g., Listeria monocytogenes, Listeria innocua, or Listeria ivanovii), Listeriaceae bacterium, a Methylocystis sp., Methylosinus trichosporium, Mobiluncus mulieris, a Neisseria sp. (e.g., Neisseria meningitidis, Neisseria bacilliformis, Neisseria cinerea, Neisseria flavescens, Neisseria lactamica, or Neisseria wadsworthii), Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, a Sphingomonas sp., Sporolactobacillus vineae, a Subdoligranulum sp., Tistrella mobilis, a Treponema sp. (e.g., Treponema denticola), and Verminephrobacter eiseniae. In some embodiments, the Cas9 polypeptide is derived from Streptococcus pyogenes (e.g., strain SF370, MGAS 10270, MGAS 10750, MGAS2096, MGAS315, MGAS5005, MGAS6180, MGAS9429, NZ131, or SSI-1), S. thermophilus (e.g., strain LMD-9), S. pseudoporcinus (e.g., strain SPIN 20026), S. mutans (e.g., strain UA 159 or NN2025), S. macacae (e.g., strain NCTC1 1558), S. gallolyticus (e.g., strain UCN34, ATCC, or BAA-2069), S. equineus (e.g., strain ATCC 9812, MGCS 124), S. dysdalactiae (e.g., strain GGS 124), S. bovis (e.g., strain ATCC 700338), S. anginosus (e.g., strain F021 1), S. agalactiae (e.g., strain NEM316 or A909), Listeria monocytogenes (e.g., strain F6854), Listeria innocua (e.g., strain Clip 11262), Enterococcus italicus (e.g., strain DSM 15952), Enterococcus faecium (e.g., strain 1_231_408), Eubacterium ventriosum, Streptococcus pasteurianus, Lactobacillus farciminis, Sphaerochaeta globus, Azospirillum (e.g., B510), Roseburia intestinalis, Nitratifractor salsuginis (e.g., strain DSM 16511), or Campylobacter lari (e.g., CF89-12).

In some embodiments, the erythroid cell further comprises a guide nucleic acid, e.g., a guide RNA, e.g., a single guide RNA (sgRNA).

In some embodiments, e.g., embodiments, of the methods of making or manufacturing described herein, b) comprises contacting the erythroid precursor cell directly with the exogenous nucleic acid-binding protein (e.g., an exogenous Cas9). In some embodiments, b) comprises contacting the erythroid precursor cell with the nucleic acid (e.g., DNA or RNA, e.g., mRNA) encoding the exogenous nucleic acid-binding protein. In some embodiments, the exogenous nucleic acid-binding protein does not have nuclease activity e.g., comprises one or more mutated nuclease domains (e.g., a nickase Cas9 that cleaves one strand of a double-stranded nucleic acid)), e.g., one or both of a mutated RuvC (e.g., D10A) and a mutated HNH (e.g., H841A) domain. In some embodiments, the first nucleic acid is not cleaved.

In some embodiments, the exogenous nucleic acid-binding protein further comprises a second domain (e.g., is a fusion protein). In some embodiments, the second domain comprises a transcriptional repressor, e.g., one or more of KRAB, MXI1, SID4x, DNMT3a, or LSD1, or a fragment or variant thereof having transcriptional repression activity, or any combination thereof. In some embodiments, the second domain comprises a transcriptional activator, e.g., one or more of VP64, p65AD, p300, Tet1, or VPR, or a fragment or variant thereof having transcriptional activation activity, or any combination thereof.

In some embodiments, the method further comprises introducing a guide nucleic acid, e.g., a guide RNA, e.g., a sgRNA into the erythroid precursor cell. In some embodiments, the sgRNA further comprises one or more RNA aptamers, e.g., one or more RNA aptamer that binds a transcriptional repressor or transcriptional activator, e.g., one or more MS2 aptamers.

In some embodiments, the first nucleic acid comprises a nucleic acid sequence encoding a gene of Table 1 or Table 2.

In some embodiments, the first nucleic acid is within 1 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, or 1000 bp (e.g., upstream of downstream) of a nucleic acid sequence encoding a gene of Table 1 or Table 2. In some embodiments, the exogenous nucleic acid-binding protein that specifically binds the first nucleic acid mediates an increase in transcription of a gene of Table 1 or Table 2, and the first nucleic acid is within 1-1000, 1-800, 1-600, 1-400, or 1-200 nucleotides upstream of the transcription start site of the gene to be upregulated, e.g., a nucleic acid sequence encoding a gene of Table 1. In some embodiments, the exogenous nucleic acid-binding protein that specifically binds the first nucleic acid mediates a decrease in transcription of a gene of Table 1 or Table 2, and the first nucleic acid is within 1-1000, 1-800, 1-600, 1-400, or 1-200 nucleotides downstream of the transcription start site of the gene to be downregulated, e.g., a nucleic acid sequence encoding a gene of Table 2. In some embodiments, the first nucleic acid is a promoter of a gene of Table 1 or Table 2. In some embodiments, the first nucleic acid is a sequence within 1 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, or 1000 bp (e.g., upstream or downstream) of a promoter of a nucleic acid sequence encoding (e.g., a promoter that is operably linked to) a gene of Table 1 or Table 2.

In some embodiments, the exogenous nucleic acid-binding protein comprises a Cpf1 protein.

In some embodiments, the first exogenous nucleic acid-binding protein binds a first target site in the first nucleic acid, and the method further comprises contacting the erythroid cell with a second exogenous nucleic acid-binding protein that specifically binds a second target site, e.g., wherein the second target site is within 100, 200, 400, 600, 800, or 1,000 bp of the first target site. In some embodiments, the method further comprises contacting the erythroid cell with a third exogenous nucleic acid-binding protein that specifically binds a third target site, e.g., wherein the third target site is within 100, 200, 400, 600, 800, or 1,000 bp of the first target site. In some embodiments, the method further comprises contacting the erythroid cell with a fourth exogenous nucleic acid-binding protein that specifically binds a fourth target site, e.g., wherein the fourth target site is within 100, 200, 400, 600, 800, or 1,000 bp of the first target site.

In some embodiments, the exogenous nucleic acid-binding protein, e.g, Cas9 polypeptide, is less than 1,300, 1,200, 1,100, or 1,000 amino acids in length.

In some embodiments, the first sequence-specific alteration in gene expression comprises making a genetic alteration to a promoter (e.g., to an endogenous promoter of a gene provided in Table 1 or Table 2), e.g., adding a promoter or portion thereof, deleting a promoter or portion thereof, or replacing a promoter or portion thereof.

In some embodiments, a nucleated precursor or progenitor cell (e.g., a hematopoietic progenitor cells) is chosen from a CD34+ selected mobilized peripheral blood cell, a cord blood cell, an induced pluripotent stem cell (iPSC), an embryonic stem cell, or an immortalized erythroblastic cell.

In some embodiments, the method further comprises culturing the modified erythroid cell under conditions allowing for expansion of the modified erythroid cell. In some embodiments, the method further comprises culturing the modified erythroid cell under conditions allowing for differentiation of the modified erythroid cell. In some embodiments, the method further comprises culturing the modified erythroid cell under conditions allowing for maturation or enucleation to provide a modified enucleated erythroid cell or a population of modified enucleated erythroid cells. In some embodiments, the method further comprises processing the population of modified enucleated erythroid cells into a pharmaceutical composition.

In some embodiments, the erythroid cell, e.g., enucleated erythroid cell has a half life which is less than 90%, 80%, 70%, 60%, 50%, 40%, 30%, 20%, or 10% of the half life of an otherwise similar unmodified erythroid cell. In some embodiments, the half-life is measured by administering a population of the cells to a subject, measuring the level of cells at one or more timepoints after administration, and calculating the amount of time taken for the number of cells detectable to decline to half the original level. In some embodiments, the erythroid cell, e.g., enucleated erythroid cell has an immunogenicity which is at least 10%, 20%, 50%, 2-fold, 5-fold, or 10-fold greater than the immunogenicity of an otherwise similar unmodified erythroid cell, e.g., as measured in a T cell proliferation assay. In some embodiments, the erythroid cell further comprises an exogenous antigen, e.g., an autoimmune antigen, e.g., selected from Table 4 or an antigenic fragment thereof. In some embodiments, the antigen is intracellular. In some embodiments, the antigen is present at the surface of the erythroid cell, e.g., wherein the exogenous antigen is fused to a transmembrane domain. In some embodiments, the erythroid cell, e.g., enucleated erythroid cell has an immunogenicity which is less than 90%, 80%, 70%, 60%, 50%, 40%, 30%, 20%, or 10% of the immunogenicity of an otherwise similar unmodified erythroid cell, e.g., in a T cell proliferation assay. In some embodiments, the erythroid cell, e.g., enucleated erythroid cell binds to CD2 (e.g., CD2 expressed on a T cell) less than an otherwise similar unmodified erythroid cell binds to CD2 (e.g., CD2 expressed on a T cell).

In some embodiments, the erythroid cell substantially lacks endogenous CD47 or CD47-encoding RNA. In some embodiments, the cell was made by a process comprising genetically modifying the DNA locus encoding CD47, e.g., by deleting the gene for CD47 or a portion thereof. In some embodiments, the process comprises contacting an erythroid precursor cell with: i) an exogenous site-specific DNA binding protein, e.g., a site-specific nuclease, e.g., a Cas9 polypeptide, and ii) a guide RNA that binds a gene encoding CD47 or a region within 100, 200, 500, 1,000, or 2,000 nucleotides of the gene encoding CD47.

In some embodiments, the differentiation factor is one that promotes differentiation to a leukocyte lineage, promotes apoptosis, promotes a fetal phenotype (e.g., expression of fetal hemoglobin), or promotes self-renewal. In some embodiments, the differentiation factor is a transcription factor. In some embodiments, the differentiation factor is one that promotes differentiation to an erythrocyte lineage, promotes cell survival, promotes self-renewal, or promotes an adult phenotype (e.g., expression of adult hemoglobin).

In some embodiments, an erythroid cell (e.g., enucleated erythroid cell) described herein is not from a subject having a hemoglobinopathy, e.g., sickle cell disease or sickle cell trait; or a thalassemia (e.g., alpha thalassemia or beta thalassemia). In some embodiments, an erythroid cell (e.g., enucleated erythroid cell) described herein has wild-type levels and/or sequence of all hemoglobins. In some embodiments, an erythroid cell (e.g., enucleated erythroid cell) described herein is from a subject that does not have a blood disorder or a hematologic condition. In some embodiments, the subject does not have one or more of (e.g., any of) a hemophilia, a SCID (e.g., SCID-X1 or ADA-SCID), a tyrosinemia (e.g., hereditary tyrosinemia), a thalassemia (e.g., beta-thalassemia), Wiskott-Aldrich syndrome, an anemia (e.g., Fanconi anemia), a leukodystrophy (e.g., adrenoleukodystrophy, metachromatic leukodystrophy, or Krabbe disease), an immunodeficiency (e.g., HIV/AIDS), a lysosomal storage disorder, alpha-mannosidosis, a neoplastic disorder of the blood (e.g., polycythemia vera), or a thrombocythaemia (e.g., familial essential thrombocythaemia).

In some embodiments, an erythroid cell is made by a process described herein, e.g., a process comprising:

providing an erythroid precursor cell encoding CD47, CD58, CR1, or a differentiation factor, and

contacting the erythroid precursor cell with an siRNA, shRNA, miRNA, or nucleic acid encoding a Cas9 polypeptide (and optionally also a gRNA), ZF polypeptide (e.g., ZFN), or TALE polypeptide (e.g., TALEN).

Gene Editing Composition and Methods

The present disclosure provides, in some aspects, a method of making or manufacturing a modified erythroid cell comprising:
a) providing an erythroid precursor cell comprising nucleic acid, e.g., endogenous nucleic acid, e.g., chromosomal nucleic acid, e.g., a cell comprising a nucleus; and
b) contacting the erythroid precursor cell with a reagent that mediates a first sequence-specific modification of the nucleic acid under conditions that allow for sequence-specific modification of the nucleic acid, to provide a modified erythroid cell,

thereby manufacturing a modified erythroid cell.

The present disclosure also provides, in some aspects, a method of making or manufacturing a modified erythroid cell comprising:
a) providing an erythroid precursor cell comprising nucleic acid, e.g., endogenous nucleic acid, e.g., chromosomal nucleic acid, e.g., a cell comprising a nucleus; and
b) contacting the erythroid precursor cell with a reagent that mediates a first sequence-specific modification of an endogenous sequence of the nucleic acid under conditions that allow for sequence-specific modification of the nucleic acid, wherein the first sequence-specific modification results in an alteration in the expression (e.g., increased expression or decreased expression) of the endogenous sequence to provide a modified erythroid cell,

thereby manufacturing a modified erythroid cell.

The present disclosure also provides, in some aspects, a method of making or manufacturing a modified erythroid cell comprising:
a) providing an erythroid precursor cell comprising nucleic acid, e.g., endogenous nucleic acid, e.g., chromosomal nucleic acid, e.g., a cell comprising a nucleus; and
b) contacting the erythroid precursor cell with a reagent that mediates a first sequence-specific modification of an endogenous sequence of the nucleic acid under conditions that allow for sequence-specific modification of the nucleic acid, wherein the first sequence-specific modification results in an alteration in the structure of an endogenous protein or RNA sequence to provide a modified erythroid cell,

thereby manufacturing a modified erythroid cell.

The present disclosure also provides, in some aspects, an erythroid cell comprising a reagent that mediates a sequence-specific modification of a nucleic acid in the erythroid cell.

The present disclosure also provides, in some aspects, a reaction mixture comprising an erythroid cell and a reagent that mediates a sequence-specific modification of a nucleic acid in the erythroid cell, e.g., wherein the reagent is inside the erythroid cell or outside the erythroid cell.

The present disclosure also provides, in some aspects, an erythroid cell comprising a sequence-specific break, e.g., a single stranded or double stranded break, in a nucleic acid in the erythroid cell.

The present disclosure also provides, in some aspects, a population of erythroid cells, wherein a plurality of erythroid cells in the population (e.g., 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99%) comprise a sequence-specific break or a break in the same region of the genome, e.g., a single stranded or double stranded break, in a nucleic acid in the erythroid cell.

The present disclosure also provides, in some aspects, a population of erythroid cells comprising:
a) a plurality of nucleated erythroid precursor cells that comprises an alteration to an endogenous gene or a non-coding region that regulates expression of the gene; and
b) a plurality of enucleated erythroid cells having altered level or structure of a gene product (e.g., a protein) of the gene.

The present disclosure also provides, in some aspects, a method of supplying a product to a subject comprising,

administering a modified erythroid cell or a population thereof as described herein to the subject,

thereby supplying a product.

The present disclosure also provides, in some aspects, a pharmaceutical composition comprising a population of modified enucleated erythroid cells comprising nucleic acids that have been contacted with a reagent that mediates a first sequence-specific modification of the nucleic acid under conditions that allow for sequence-specific modification of the nucleic acid,

wherein the enucleated erythroid cell has an alteration in the expression (e.g., increased expression or decreased expression) of or the structure of the endogenous sequence compared to a cell that has not been contacted with the reagent.

The following embodiments can apply to any of the aspects herein, e.g., any of the compositions and methods herein above.

In some embodiments, e.g., embodiments of the methods of making or manufacturing a modified erythroid cell described herein, the reagent that mediates the first sequence-specific modification specifically binds a recognition sequence of at least 10, 12, 14, 16, 18, 20, 25, or 30 nucleotides in the nucleic acid, e.g., endogenous nucleic acid, e.g., chromosomal nucleic acid. In some embodiments, the reagent that mediates the first sequence-specific modification specifically binds a non-palindromic recognition sequence in the nucleic acid, e.g., endogenous nucleic acid, e.g., chromosomal nucleic acid. In some embodiments, the reagent that mediates the first sequence-specific modification is other than a restriction endonuclease.

Various culture conditions, e.g., for modified erythroid cells, are contemplated. In some embodiments, e.g., embodiments of the methods of making or manufacturing a modified erythroid cell described herein, the method further comprises culturing the modified erythroid cell under conditions allowing for expansion of the modified erythroid cell. In some embodiments, the method further comprises culturing the modified erythroid cell under conditions allowing for differentiation of the modified erythroid cell. In certain embodiments, the method further comprises culturing the modified erythroid cell under conditions allowing for maturation and/or enucleation to provide a population of modified enucleated erythroid cells. In certain embodiments, the method further comprises processing the population of modified enucleated erythroid cells into a pharmaceutical composition.

In some embodiments, e.g., embodiments of the methods of making or manufacturing a modified erythroid cell described herein,
a) the erythroid precursor cell is wild-type, e.g., is wild-type at all hemoglobin loci, e.g., does not carry a mutation for sickle cell disease, sickle cell trait or a thalassemia (e.g., alpha thalassemia or beta thalassemia);
b) the sequence-specific modification does not correct a mutation;
c) the sequence-specific modification confers a phenotype on the modified erythroid cell that is not present on an otherwise similar wild-type erythroid cell;
d) the sequence-specific modification is made to a wild-type endogenous sequence (e.g., coding region or promoter) in the erythroid precursor cell; and/or
e) the sequence-specific modification is made to an endogenous sequence other than a globin, albumin, CCR5, CXCR4, AAVS1, Rosa, HPRT, BCL11A, or KLF1.

In some embodiments, e.g., embodiments of the methods of making or manufacturing a modified erythroid cell described herein, the first sequence-specific modification is at a first site that comprises:
a) a sequence that encodes a transcribed or translated product, e.g., a sequence that encodes a polypeptide;
b) a sequence that modulates the expression or structure of a second sequence, e.g., the modification is in a control element, e.g., a promoter, an enhancer, or an insulator; or
c) a sequence that binds a polypeptide, e.g., a DNA-binding protein (e.g., a transcription factor or a histone) or an enzyme (e.g., a kinase, a phosphatase, a lyase, a hydrolase, a fucosyltransferase, or a glycosyltransferase).

In some embodiments, e.g., embodiments of the methods of making or manufacturing a modified erythroid cell described herein, the first sequence-specific modification:
a) alters an endogenous nucleic acid sequence (e.g., alters 1-2, 2-5, 5-10, 10-20, 20-50, 50-100, 100-200, 200-500, or 500-1000 nucleotides);
b) comprises a deletion of one or more nucleotides (e.g., of 1-2, 2-5, 5-10, 10-20, 20-50, 50-100, 100-200, 200-500, or 500-1000 nucleotides);
c) comprises an insertion of one or more nucleotides (e.g., of 1-2, 2-5, 5-10, 10-20, 20-50, 50-100, 100-200, 200-500, or 500-1000 nucleotides);
d) comprises an inversion of one or more nucleotides (e.g., of 1-2, 2-5, 5-10, 10-20, 20-50, 50-100, 100-200, 200-500, or 500-1000 nucleotides); or
e) comprises a substitution of one or more nucleotides (e.g., of 1-2, 2-5, 5-10, 10-20, 20-50, 50-100, 100-200, 200-500, or 500-1000 nucleotides).

In some embodiments, e.g., embodiments of the methods of making or manufacturing a modified erythroid cell described herein, the first sequence-specific modification:
a) modifies the expression of a sequence (e.g., a gene provided in Table 1 or Table 2), e.g., increases or decreases the expression of the sequence; and/or
b) alters the sequence of an expressed product, e.g., an RNA or polypeptide, e.g., by a point mutation, altering an activity of the product, truncating the product, or decreasing immunogenicity of the product.

In some embodiments, e.g., embodiments of the methods of making or manufacturing a modified erythroid cell described herein, the first sequence-specific modification results in an alteration in the expression of or the structure of a polypeptide, e.g., a cytokine, an enzyme (e.g., a kinase, a phosphatase, a lyase, a hydrolase, a fucosyltransferase, or a glycosyltransferase), a cytoplasmic protein (e.g., hemoglobin), a transmembrane protein (e.g., a type I transmembrane protein (e.g., EPO Receptor, glycophorin A (GPA), complement receptor 1 (CR1), CD45, or CD46), a type II transmembrane protein (e.g., CD71 or Ke11), type III transmembrane protein (e.g., CD47, CD117, glucose transporter type 1 (GLUT1), or flippase), a membrane transport protein (e.g., an ion channel or an anion exchanger (e.g., BAND III)), a soluble protein (e.g., a secreted or intracellular protein (e.g., CD59)), a membrane protein (e.g., CD55), an antigen (e.g., blood group antigen, e.g., an A, B, O, H, or Rh antigen), a cytoskeletal protein (e.g., spectrin), or an adaptor protein (e.g., an ankyrin).

In some embodiments, e.g., embodiments of the methods of making or manufacturing a modified erythroid cell described herein, the first sequence-specific modification results in an increase in the expression of a product, e.g., an RNA or polypeptide. In some embodiments, e.g., embodiments of the methods of making or manufacturing a modified erythroid cell described herein, the first sequence-specific modification results in a decrease in the expression of a product, e.g., an RNA or polypeptide.

In some embodiments, e.g., embodiments of the methods of making or manufacturing a modified erythroid cell described herein, the modification results in an alteration in the expression of, or the structure of, a polypeptide selected from Table 1 or Table 2.

Various modifications can be made to, e.g., the nucleic acids (e.g., the endogenous nucleic acids) described herein. In some embodiments, e.g., embodiments of the methods of making or manufacturing a modified erythroid cell described herein, the modification results in the expression of an exogenous product, e.g., an exogenous RNA or polypeptide. In some embodiments, e.g., embodiments of the methods of making or manufacturing a modified erythroid cell described herein, the modification does not result in the expression of an exogenous product, e.g., an exogenous RNA or polypeptide. In certain embodiments, the modification inserts an exogenous DNA into the erythroid precursor cell nucleus. In certain embodiments, the exogenous protein comprises a cytokine, an enzyme (e.g., a kinase, a phosphatase, a lyase, a hydrolase, a fucosyltransferase, or a glycosyltransferase), a cytoplasmic protein, a transmembrane protein (e.g., a type 1, a type II, or type III transmembrane protein), a membrane transport protein (e.g., an ion channel or an anion exchanger), a soluble protein (e.g., a secreted or intracellular protein), a membrane protein, an antigen, a cytoskeletal protein, or an adaptor protein.

In some embodiments, e.g., embodiments of the methods of making or manufacturing a modified erythroid cell described herein, the method further comprises making a second sequence-specific modification of the nucleic acid. In certain embodiments, the first sequence-specific modification is disposed in a first site, e.g., a first coding region, first promoter region, first enhancer region, first insulator region, or first chromosome, and the second sequence-specific modification is disposed in the first site. In certain embodiments, the first sequence-specific modification is disposed in a first site, e.g., a first coding region, first promoter region, first enhancer region, first insulator region, or first chromosome, and the second sequence-specific modification is disposed in a second site, e.g., a second coding region, second promoter region, second enhancer region, second insulator region, or second chromosome.

In certain embodiments, the second site comprises:
a) a sequence that encodes a transcribed or translated product, e.g., a sequence that encodes a polypeptide;
b) a sequence that modulates the expression or structure of a third sequence, e.g., the modification is in a control element, e.g., a promoter, enhancer, or insulator; or
c) a sequence that binds a polypeptide, e.g., a DNA-binding protein (e.g., a transcription factor or a histone) or an enzyme (e.g., a kinase, a phosphatase, a lyase, a hydrolase, a fucosyltransferase, or a glycosyltransferase).

In certain embodiments,
a) the first sequence-specific modification increases expression of a first sequence, e.g., a first endogenous sequence, and the second sequence-specific modification increases expression of a second sequence, e.g., a second endogenous sequence;
b) the first sequence-specific modification decreases expression of a first sequence, e.g., a first endogenous sequence, and the second sequence-specific modification decreases expression of a second sequence, e.g., a second endogenous sequence;
c) the first sequence-specific modification increases expression of a first sequence, e.g., a first endogenous sequence, and the second sequence-specific modification decreases expression of a second sequence, e.g., a second endogenous sequence; or
d) the first sequence-specific modification decreases expression of a first sequence, e.g., a first endogenous sequence, and the second sequence-specific modification increases expression of a second sequence, e.g., a second endogenous sequence.

In certain embodiments,
a) the first sequence-specific modification is disposed in a coding region and the second sequence-specific modification is disposed in a non-coding region, e.g., a promoter region, an enhancer region, or an insulator region; or
b) the first sequence-specific modification is disposed in a non-coding region, e.g., a promoter region, an enhancer region, or an insulator region, and the second sequence-specific modification is disposed in a coding region.

In certain embodiments, the second sequence-specific modification:
a) alters the endogenous sequence (e.g., alters 1-2, 2-5, 5-10, 10-20, 20-50, 50-100, 100-200, 200-500, or 500-1000 nucleotides);
b) comprises a deletion of one or more nucleotides (e.g., of 1-2, 2-5, 5-10, 10-20, 20-50, 50-100, 100-200, 200-500, or 500-1000 nucleotides);
c) comprises an insertion of one or more nucleotides (e.g., of 1-2, 2-5, 5-10, 10-20, 20-50, 50-100, 100-200, 200-500, or 500-1000 nucleotides);
d) comprises an inversion of one or more nucleotides (e.g., of 1-2, 2-5, 5-10, 10-20, 20-50, 50-100, 100-200, 200-500, or 500-1000 nucleotides); or
e) comprises a substitution of one or more nucleotides (e.g., of 1-2, 2-5, 5-10, 10-20, 20-50, 50-100, 100-200, 200-500, or 500-1000 nucleotides).

In certain embodiments, the second sequence-specific modification:
a) modifies the expression of a sequence, e.g., increases or decreases the expression of the sequence; and/or
b) alters the sequence of an expressed product, e.g., an RNA or polypeptide, e.g., by a point mutation, altering activity of the product, truncating the product, or decreasing immunogenicity of the product.

The second sequence-specific modification may, in some embodiments, result in a functional effect. In certain embodiments, the second sequence-specific modification results in an alteration in the expression of or the structure of a polypeptide, e.g., a cytokine, an enzyme (e.g., a kinase, a phosphatase, a lyase, a hydrolase, a fucosyltransferase, or a glycosyltransferase), a cytoplasmic protein (e.g., hemoglobin), a transmembrane protein (e.g., a type I transmembrane protein (e.g., EPO Receptor, glycophorin A (GPA), complement receptor 1 (CR1), CD45, or CD46), a type II transmembrane protein (e.g., CD71 or Ke11), type III transmembrane protein (e.g., CD47, CD117, glucose transporter type 1 (GLUT1), or flippase), a membrane transport protein (e.g., an ion channel or an anion exchanger (e.g., BAND III)), a soluble protein (e.g., a secreted or intracellular protein (e.g., CD59)), a membrane protein (e.g., CD55), an antigen (e.g., blood group antigen, e.g., an A, B, O, H, or Rh antigen), a cytoskeletal protein (e.g., spectrin), or an adaptor protein (e.g., an ankyrin). In certain embodiments, the second sequence-specific modification results in an increase in the expression of a product, e.g., an RNA or polypeptide. In certain embodiments, the second sequence-specific modification results in a decrease in the expression of a product, e.g., an RNA or polypeptide. In certain embodiments, the second sequence-specific modification results in an alteration in the expression of or the structure of a polypeptide selected from Table 1 or Table 2.

In some embodiments, e.g., embodiments of the methods of making or manufacturing a modified erythroid cell described herein, the reagent comprises a CRISPR component, e.g., a Cas9 polypeptide, e.g., a Cas9 polypeptide having nuclease activity or a catalytically inactive Cas9 polypeptide, and optionally a guide nucleic acid (e.g., a gRNA). Various CRISPR components are available. In certain embodiments, the CRISPR component covalently modifies the nucleic acid. In certain embodiments, the CRISPR component cleaves the nucleic acid. In certain embodiments, the CRISPR component attaches an atom to the nucleic acid or a protein bound thereto, e.g., methylates the nucleic acid or acetylates a histone bound to the nucleic acid. In certain embodiments, the CRISPR component comprises a guide nucleic acid, e.g., a guide RNA, e.g., a single guide RNA. In certain embodiments, the CRISPR component modifies a nucleic acid encoding a polypeptide, e.g., a polypeptide of Table 1 or Table 2, or a non-coding sequence (e.g., a promoter) that regulates expression of the polypeptide. In certain embodiments, the CRISPR component modifies an endogenous nucleic acid encoding a polypeptide selected from: a cytokine, an enzyme (e.g., a kinase, a phosphatase, a lyase, a hydrolase, a fucosyltransferase, or a glycosyltransferase), a cytoplasmic protein (e.g., hemoglobin), a transmembrane protein (e.g., a type I transmembrane protein (e.g., EPO Receptor, glycophorin A (GPA), complement receptor 1 (CR1), CD45, or CD46), a type II transmembrane protein (e.g., CD71 or Ke11), type III transmembrane protein (e.g., CD47, CD117, glucose transporter type 1 (GLUT1), or flippase), a membrane transport protein (e.g., an ion channel or an anion exchanger (e.g., BAND III)), a soluble protein (e.g., a secreted or intracellular protein (e.g., CD59)), a membrane protein (e.g., CD55), an antigen (e.g., blood group antigen, e.g., an A, B, O, H, or Rh antigen), a cytoskeletal protein (e.g., spectrin), and an adaptor protein (e.g., an ankyrin).

In some embodiments, e.g., embodiments of the methods of making or manufacturing a modified erythroid cell described herein, the reagent comprises a vector component, e.g., a viral vector component, e.g., a DNA or RNA viral vector component, e.g., an adeno-associated virus (AAV) vector component, e.g., a self-complementary adeno-associated virus (scAAV) comprising double-stranded DNA vector component. Various viral vector components are available. In certain embodiments, the viral vector component, e.g., AAV vector component, comprises an exogenous nucleic acid sequence, e.g., an exogenous nucleic acid sequence flanked by inverted terminal repeats (ITRs). In certain embodiments, the viral vector component, e.g., AAV vector component, further comprises one or more nucleic acid sequences encoding one or more Rep proteins and/or one or more nucleic acid sequences encoding one or more Cap proteins, e.g., in trans to the exogenous nucleic acid sequence. In certain embodiments, the exogenous nucleic acid sequence encodes a polypeptide selected from: a cytokine, an enzyme (e.g., a kinase, a phosphatase, a lyase, a hydrolase, a fucosyltransferase, or a glycosyltransferase), a cytoplasmic protein, a transmembrane protein (e.g., a type I, a type II, or type III transmembrane protein), a membrane transport protein (e.g., an ion channel or an anion exchanger), a soluble protein (e.g., a secreted or intracellular protein), a membrane protein, an antigen, a cytoskeletal protein, or an adaptor protein. In certain embodiments, the exogenous nucleic acid sequence encodes an expressed product selected from: a CRISPR component, e.g., Cas9 polypeptide and/or a guide nucleic acid, a Zinc Finger nuclease (ZFN) component, and a transcription activator-like effector nuclease (TALEN) component. In certain embodiments, the viral vector component, e.g., AAV vector component, replicates or is capable of replicating in the erythroid precursor cell nucleus. In certain embodiments, the viral vector component, e.g., AAV vector component, expresses or is capable of expressing the exogenous nucleic acid sequence in the erythroid precursor cell nucleus. In certain embodiments, the viral vector component, e.g., AAV vector component, inserts or is capable of inserting one or more nucleotides into the erythroid precursor cell nucleus, e.g., all nucleotides of the AAV integrate into the nucleic acid of the erythroid precursor cell nucleus. In certain embodiments, the viral vector component, e.g., AAV vector component, binds to a nucleic acid encoding a polypeptide, e.g., a polypeptide of Table 1, or a non-coding sequence (e.g., a promoter) that regulates expression of the polypeptide.

In some embodiments, e.g., embodiments of the methods of making or manufacturing a modified erythroid cell described herein, the reagent comprises a transcription activator-like effector nuclease (TALEN) component, e.g., a fusion protein comprising a TAL effector DNA binding domain and a DNA cleavage domain, e.g., a TALEN component having nuclease activity. In some embodiments, the DNA cleavage domain comprises an endonuclease, a bacterial endonuclease, a type II restriction endonuclease, or a type IIS restriction endonuclease e.g., FokI. Various TALEN components are available. In certain embodiments, the method further comprises contacting the erythroid precursor cell with a second TALEN component, e.g., a second fusion protein comprising a second TAL effector DNA binding domain and a second DNA cleavage domain. In certain embodiments, the TALEN component binds a first target DNA sequence and the second TALEN component binds a second target DNA sequence, e.g., wherein the first and second target DNA sequences are within 1, 2, 5, 10, 20, 50, or 100 nucleotides of each other. In certain embodiments, the DNA cleavage domain and the second DNA cleavage domain have increased nuclease activity when dimerized with each other. In certain embodiments, the method further comprises contacting the erythroid precursor cell with a nucleic acid, e.g., a nucleic acid comprising a first homology arm, a second homology arm, and a mutation relative to the erythroid precursor cell genome between the first homology arm and the second homology arm. In certain embodiments, the TAL effector DNA binding domain comprises a variable amino acid sequence, e.g., an amino acid sequence having specific nucleotide recognition, e.g., a Repeat Variable Diresidue (RVD). In certain embodiments, the TAL effector DNA binding domain binds to a nucleic acid encoding a polypeptide, e.g., a polypeptide of Table 1, or a non-coding sequence (e.g., a promoter) that regulates expression of the polypeptide. In certain embodiments, the DNA cleavage domain comprises one or more domains of an endonuclease, e.g., a dimer, e.g., a FokI endonuclease, e.g., a wild-type or mutated FokI endonuclease. In certain embodiments, the TALEN component cleaves the nucleic acid. In certain embodiments, the TALEN component mediates a break, e.g., a double-stranded break (DSB), in the nucleic acid. In certain embodiments, the TALEN component mediates a mutation, e.g., a frame shift mutation, in the nucleic acid. In certain embodiments, the TALEN component modifies a nucleic acid encoding a polypeptide, e.g., a polypeptide of Table 1, or a non-coding sequence (e.g., a promoter) that regulates expression of the polypeptide. In certain embodiments, the TALEN components modifies an endogenous nucleic acid encoding a polypeptide selected from: a cytokine, an enzyme (e.g., a kinase, a phosphatase, a lyase, a hydrolase, a fucosyltransferase, or a glycosyltransferase), a cytoplasmic protein (e.g., hemoglobin), a transmembrane protein (e.g., a type I transmembrane protein (e.g., EPO Receptor, glycophorin A (GPA), complement receptor 1 (CR1), CD45, or CD46), a type II transmembrane protein (e.g., CD71 or Ke11), type III transmembrane protein (e.g., CD47, CD117, glucose transporter type 1 (GLUT1), or flippase), a membrane transport protein (e.g., an ion channel or an anion exchanger (e.g., BAND III)), a soluble protein (e.g., a secreted or intracellular protein (e.g., CD59)), a membrane protein (e.g., CD55), an antigen (e.g., blood group antigen, e.g., an A, B, O, H, or Rh antigen), a cytoskeletal protein (e.g., spectrin), or an adaptor protein (e.g., an ankyrin). In certain embodiments, the TALEN component mediates the insertion of exogenous DNA into the erythroid precursor cell nucleus. In certain embodiments, the exogenous protein comprises a cytokine, an enzyme (e.g., a kinase, a phosphatase, a lyase, a hydrolase, a fucosyltransferase, or a glycosyltransferase), a cytoplasmic protein, a transmembrane protein (e.g., a type 1, a type II, or type III transmembrane protein), a membrane transport protein (e.g., an ion channel or an anion exchanger), a soluble protein (e.g., a secreted or intracellular protein), a membrane protein, an antigen, a cytoskeletal protein, or an adaptor protein.

In some embodiments, e.g., embodiments of the methods of making or manufacturing a modified erythroid cell described herein, the reagent comprises a Zinc Finger nuclease (ZFN) component, e.g., a fusion protein comprising a Zinc Finger (ZF) DNA binding domain and a DNA cleavage domain, e.g., a ZFN component having nuclease activity. Various ZFN components are available. In certain embodiments, the method further comprises contacting the erythroid precursor cell with a second ZFN component, e.g., a second fusion protein comprising a second ZF DNA binding domain and a second DNA cleavage domain. In certain embodiments, the ZFN component binds a first target DNA sequence and the second ZFN component binds a second target DNA sequence, e.g., wherein the first and second target DNA sequences are within 1, 2, 5, 10, 20, 50, or 100 nucleotides of each other. In certain embodiments, the DNA cleavage domain and the second DNA cleavage domain have increased nuclease activity when dimerized with each other. In certain embodiments, the method further comprises contacting the erythroid precursor cell with a nucleic acid, e.g., a nucleic acid comprising a first homology arm, a second homology arm, and a mutation relative to the erythroid precursor cell genome between the first homology arm and the second homology arm. In certain embodiments, the ZF DNA binding domain comprises one, two, three, four, five, six, seven, eight, nine, or more structural motifs, e.g., one or more of a Cys₂His₂, a Gag knuckle, a treble clef, a zinc ribbon, or a Zn₂/Cys₂ motif, and one or more zinc ions. In certain embodiments, the ZF DNA binding domain binds to a nucleic acid sequence of about 9 to about 18 base pairs. In certain embodiments, the ZF DNA binding domain binds to a nucleic acid encoding a polypeptide, e.g., a polypeptide of Table 1, or a non-coding sequence (e.g., a promoter) that regulates expression of the polypeptide. In certain embodiments, the DNA cleavage domain comprises one or more domains of an endonuclease, e.g., a dimer, e.g., a FokI endonuclease, e.g., a wild-type or mutated FokI endonuclease. In certain embodiments, the ZFN component cleaves the nucleic acid. In certain embodiments, the ZFN component mediates a break, e.g., a double-stranded break (DSB), in the nucleic acid. In certain embodiments, the ZFN component mediates a mutation, e.g., a frame shift mutation, in the nucleic acid. In certain embodiments, the ZFN component modifies a nucleic acid encoding a polypeptide, e.g., a polypeptide of Table 1, or a non-coding sequence (e.g., a promoter) that regulates expression of the polypeptide. In certain embodiments, the ZFN component modifies an endogenous nucleic acid encoding a polypeptide selected from: a cytokine, an enzyme (e.g., a kinase, a phosphatase, a lyase, a hydrolase, a fucosyltransferase, or a glycosyltransferase), a cytoplasmic protein (e.g., hemoglobin), a transmembrane protein (e.g., a type I transmembrane protein (e.g., EPO Receptor, glycophorin A (GPA), complement receptor 1 (CR1), CD45, or CD46), a type II transmembrane protein (e.g., CD71 or Ke11), type III transmembrane protein (e.g., CD47, CD117, glucose transporter type 1 (GLUT1), or flippase), a membrane transport protein (e.g., an ion channel or an anion exchanger (e.g., BAND III)), a soluble protein (e.g., a secreted or intracellular protein (e.g., CD59)), a membrane protein (e.g., CD55), an antigen (e.g., blood group antigen, e.g., an A, B, O, H, or Rh antigen), a cytoskeletal protein (e.g., spectrin), and an adaptor protein (e.g., an ankyrin). In certain embodiments, the TALEN component mediates the insertion of exogenous DNA into the erythroid precursor cell nucleus. In certain embodiments, the exogenous protein comprises a cytokine, an enzyme (e.g., a kinase, a phosphatase, a lyase, a hydrolase, a fucosyltransferase, or a glycosyltransferase), a cytoplasmic protein, a transmembrane protein (e.g., a type 1, a type II, or type III transmembrane protein), a membrane transport protein (e.g., an ion channel or an anion exchanger), a soluble protein (e.g., a secreted or intracellular protein), a membrane protein, an antigen, a cytoskeletal protein, or an adaptor protein. In certain embodiments, contacting the erythroid precursor cell with the reagent comprises performing lentiviral transduction, RNA electroporation, or RNA transfection (e.g., using lipid nanoparticles).

In some embodiments, the present disclosure provides a population of modified erythroid cells made, e.g., by any of the methods of making or manufacturing a modified erythroid cell described herein. In certain embodiments, the population comprises one or more enucleated erythroid cells. In certain embodiments, at least 60%, 65%, 70%, 75%, 80%, 85%, 90%, 91%, 92%, 93%, 94%, 95%, 96%, 97%, 98%, or 99% of the erythroid cells in the population are enucleated. In certain embodiments, the population comprises erythroid cells having modified levels, e.g., increased levels, e.g., increased as compared with an otherwise similar but non-modified cell, of a polypeptide selected from Table 1 or Table 2.

In some embodiments, e.g., embodiments of the reaction mixtures described herein, the reaction mixture further comprises a CRISPR component, e.g., a CRISPR component as described herein. In some embodiments, e.g., embodiments of the reaction mixtures described herein, the reaction mixture further comprises an AAV component, e.g., an AAV component as described herein. In some embodiments, e.g., embodiments of the reaction mixtures described herein, the reaction mixture further comprises a TALEN component, e.g., a TALEN component as described herein. In some embodiments, e.g., embodiments of the reaction mixtures described herein, the reaction mixture further comprises a ZFN component, e.g., a ZFN component as described herein.

In some embodiments, e.g., embodiments of the methods of supplying a product to a subject described herein, the modified erythroid cell is allogeneic or autologous to the subject.

The disclosure contemplates all combinations of any one or more of the foregoing aspects and/or embodiments, as well as combinations with any one or more of the embodiments set forth in the detailed description and examples.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Dec. 29, 2017. When one gene or protein references a plurality of sequence accession numbers, all of the sequence variants are encompassed.

### BRIEF DESCRIPTION OF THE DRAWINGS

FIG. 1 is a bar graph depicting CD47 surface staining of erythroid cells measured by fluorescence-activated cell sorting. The x axis indicates the sample (Cas9-only controls and experimental samples treated with Cas9 and CD47 sgRNA). The y axis indicates the percentage of cells in the sample that are positive for FITC staining, as a measure of CD47 surface expression. Black bars indicate cells 2 days after electroporation, and white bars indicate cells 7 days after electroporation. This figure indicates that CD47 knockout enucleated cells were generated.

FIGS. 2A and 2B are bar graphs depicting the level at which CD47+ or CD47− enucleated erythroid cells are adhered to and/or engulfed by macrophages. In both figures, the x axis indicates the sample (Cas9-only controls which are CD47+ and experimental samples treated with Cas9 and CD47 sgRNA which are CD47−). The y axis indicates the percent of enucleated erythroid cells associated with macrophages. FIG. 2A shows attachment and/or engulfment by macrophages that are not stimulated by LPS. The two asterisks indicate that CD47− enucleated erythroid cells are engulfed by the non-activated macrophages at a statistically significantly higher level (p<0.05) than CD47+ control cells. FIG. 2B shows attachment and/or engulfment by LPS-stimulated macrophages. The two asterisks indicate that CD47− enucleated erythroid cells are engulfed by the LPS-stimulated macrophages at a statistically significantly higher level (p<0.05) than CD47+ control cells. These figures indicate that CD47 knockout enucleated erythroid cells more readily attach and/or are engulfed by non-LPS-stimulated macrophages and by LPS-stimulated macrophages.

### DETAILED DESCRIPTION OF THE INVENTION

### Definitions

An “adeno-associated virus (AAV) vector” as used herein refers to a vector derived from an adeno-associated virus and capable of site-specific homologous recombination. In some embodiments, the AAV vector comprises, from 5′ to 3′, a first inverted terminal repeat, a first homology arm, a sequence of interest, a second homology arm, and a second inverted terminal repeat.

A “CRISPR component” as used herein refers to a CRISPR polypeptide (e.g., a Cas9 polypeptide) or a nucleic acid (e.g., DNA or RNA) encoding the same.

A “CRISPR polypeptide” as used herein refers to a CRISPR/Cas endonuclease or a derivative thereof (from class I, II, or III), e.g., a nuclease-dead derivative. In some embodiments, the CRISPR polypeptide is a Class I CRISPR/Cas endonuclease or a derivative thereof. In some embodiments, the CRISPR polypeptide is a type II CRISPR/Cas endonuclease, such as Cas9, or a derivative thereof. In some embodiments, the CRISPR polypeptide is a type III CRISPR/Cas endonuclease. In some embodiments, the CRISPR polypeptide is a Type III-B Cmr complex, e.g., a Type III-B Cmr complex derived from Pyrococcus furiosus, Sulfolobus solfataricus, or Thermus thermophilus. In some embodiments, the CRISPR polypeptide is a type V CRISPR/Cas endonuclease, such as Cpf1, or a derivative thereof. See, e.g., Hale, C. R. et al. Genes & Development, 2014, 28:2432-2443, and Makarova K. S. et al. Nature Reviews Microbiology, 2015, 13, 1-15.

A “Cas9 polypeptide” refers to a polypeptide which has sequence-specific nucleic acid binding activity in the presence of (e.g., complexed with) a guide RNA, and which comprises an amino acid sequence having at least 4 motifs which have at least 75%, 80%, 85%, 90%, 95%, 96%, 97%, 98%, or 99% amino acid sequence identity, or 100% percent amino acid sequence identity, to the motifs 1, 2, 3, and 4 of the Cas9 amino acid sequence of any of SEQ ID NOs:1-4 herein, or to the corresponding portions in any of the amino acid sequences set forth in SEQ ID NOs:1-829 of US Pat. App. Pub. 20170137801, which is herein incorporated by reference in its entirety. In some embodiments, the Cas9 polypeptide comprises an amino acid sequence having at least 75%, 80%, 85%, 90%, 95%, 96%, 97%, 98%, or 99% amino acid sequence identity, or 100% percent amino acid sequence identity, to the amino acids at positions 7 to 166 or 731 to 1003 of SEQ ID NO:8 of US Pat. App. Pub. 20170137801, or the corresponding amino acids of those set forth in SEQ ID NOs:1-7 or 9-829 of US Pat. App. Pub. 20170137801. In some embodiments, the Cas9 polypeptide is derived from S. aureus, S. pneumoniae, S. pyogenes, S. thermophilus, N. meningitidis or Acidovorax ebreus. In some embodiments, the Cas9 polypeptide comprises an HNH homing endonuclease domain and a split RuvC/RNaseH endonuclease domain whereby each Cas9 polypeptide shares 4 primary motifs: motifs 1, 2, and 4, which are RuvC like motifs and motif 3, which is an HNH motif. For Streptococcus pyogenes, motif 1 is SEQ ID NO:1 herein, motif 2 is SEQ ID NO:2 herein, motif 3 is SEQ ID NO:3 herein, and motif 4 is SEQ ID NO:4 herein. In some embodiments, the Cas9 polypeptide comprises a nuclear localization sequence (NLS).

“Derived from” as that term is used herein, indicates a relationship between a first and a second molecule. It generally refers to structural similarity between the first molecule and a second molecule and does not connote or include a process or source limitation on a first molecule that is derived from a second molecule.

As used herein, “differentiation factor” refers to a molecule (e.g., protein, gene, or RNA) that influences differentiation, e.g., that inhibits differentiation, or promotes differentiation along a lineage.

As used herein, “enucleated” refers to a cell, e.g., a reticulocyte or mature red blood cell, that lacks a nucleus. In some embodiments, an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In some embodiments, an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell.

“Erythroid cell” as used herein, includes a nucleated red blood cell, a red blood cell precursor, an enucleated mature red blood cell, and a reticulocyte. For example, any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al. (2014), Mol. Ther. 22(2): 451-463). In addition, the cells may be intended for autologous use or provide a source for allogeneic transfusion. In some embodiments, erythroid cells are cultured. In some embodiments, an erythroid cell is an enucleated red blood cell.

As used herein, the terms “endogenous polypeptide” and “endogenous protein” are used interchangeably to refer to a polypeptide that is produced by a wild-type cell of a given type or tissue.

As used herein, the term “endogenous DNA” refers to DNA that is present in the genome of a wild-type cell of a given type. The genome may be, e.g., the nuclear genome. In some embodiments, the endogenous DNA is transcribed in a cell of a given type or tissue, and in other embodiments, the endogenous DNA is not transcribed in a wild-type cell of that type or tissue.

As used herein, the term “endogenous RNA” refers to RNA that is expressed by a wild-type cell of a given type or tissue.

As used herein, the term “endogenous nucleic acid” refers to a nucleic acid that is present in a wild-type cell of a given type or tissue, e.g., an endogenous DNA or endogenous RNA as described herein.

As used herein, the term “exogenous polypeptide” and “exogenous protein” are used interchangeably to refer to a polypeptide that is not produced by a wild-type cell of a given type or is present at a lower level in a wild-type cell than in a cell containing the exogenous polypeptide. In some embodiments, an exogenous polypeptide is a polypeptide encoded by a nucleic acid that was introduced into the cell, which nucleic acid is optionally not retained by the cell. In some embodiments, an exogenous polypeptide is a polypeptide conjugated to the surface of the cell by chemical or enzymatic means.

“Genetically engineered cell,” as used herein, is a cell that comprises a nucleic acid sequence (e.g., DNA or RNA, e.g., mRNA) that is not present in, or is present at a different level than, an otherwise similar cell under similar conditions that is not engineered (an exogenous nucleic acid), or a cell that comprises a polypeptide expressed from said nucleic acid sequence. In some embodiments, a genetically engineered cell comprises an exogenous nucleic acid (e.g., DNA or RNA, e.g., mRNA). In some embodiments, a genetically engineered cell comprises an exogenous protein expressed from the exogenous nucleic acid that was present in a precursor of the cell, but did not retain some or all of the exogenous nucleic acid. For instance, in some embodiments, the exogenous nucleic acid is lost over the course of differentiation, e.g., due to enucleation or nucleic acid degradation. In some embodiments, the exogenous nucleic acid sequence comprises a chromosomal or extra-chromosomal exogenous nucleic acid sequence that comprises a sequence which is expressed as RNA, e.g., mRNA. In some embodiments, the exogenous nucleic acid sequence comprises a chromosomal or extra-chromosomal nucleic acid sequence that comprises a sequence which encodes a polypeptide or which is expressed as a polypeptide.

As used, herein, a “modified erythroid cell” refers to an erythroid cell that differs in at least one characteristic from an otherwise similar unmodified erythroid cell. In some embodiments, the modified erythroid cell comprises an elevated or reduced level of an endogenous protein or RNA compared to the otherwise similar unmodified erythroid cell. In some embodiments, the modified erythroid cell comprises an exogenous protein, RNA, or DNA.

As used herein, the term “otherwise similar”, when used comparing two cells, refers to two cells of the same tissue and stage of differentiation, from the same subject, cultured similarly.

As used herein, the terms “peptide,” “polypeptide,” and “protein” are used interchangeably to refer to any chain of two or more natural or unnatural amino acid residues, regardless of post-translational modification (e.g., glycosylation or phosphorylation), constituting all or part of a naturally-occurring or non-naturally occurring polypeptide or peptide.

A “guide RNA” refers to a polynucleotide sequence having sufficient complementarity with a target nucleic acid (e.g., an endogenous nucleic acid) sequence to hybridize with the target nucleic acid and direct sequence-specific binding of a CRISPR polypeptide to the target nucleic acid. In some embodiments, the guide RNA comprises at least 8, 9, 10, 11, 12, 13, 14, 15, 16, 17, 18, 19, 20, 21, 22, 23, 24, 25, 26, 27, 28, 29, or 30 nucleotides. In some embodiments, the guide RNA has at least 50%, 60%, 70%, 75%, 80%, 85%, 86%, 87%, 88%, 89%, 90%, 91%, 92%, 93%, 94%, 95%, 96%, 97%, 98%, or 99%, identity, or 100% identity, to the reverse complement of its target nucleic acid. In some embodiments, the guide RNA is a dual guide RNA, e.g., a first molecule that comprises a CRISPR RNA (crRNA) and a second molecule that comprises a trans-activating crRNA (tracrRNA). In other embodiments, the guide RNA is a single guide RNA (sgRNA).

A “single guide RNA” as used herein refers to a single polynucleotide that comprises (i) a portion with sufficient complementarity with a target nucleic acid to hybridize with the target nucleic acid and (ii) a portion that binds to and directs a CRISPR polypeptide (e.g., a Cas9 polypeptide or a Cpf1 polypeptide) to the target nucleic acid. See, e.g., Jinek et al., Science 2012; Briner et al., Mol. Cell 2014.

A “transcription activator-like effector (TALE) component” as used herein refers to a TALE polypeptide or a nucleic acid (e.g., DNA or RNA) encoding the same.

A “transcription activator-like effector (TALE) polypeptide” as used herein refers to a polypeptide comprising a site-specific transcription activator-like (TAL) effector domain. An example of a TALE polypeptide is a TALEN polypeptide. A TALE polypeptide may further comprise a transcriptional activator or repressor domain.

A “transcription activator-like effector nuclease (TALEN) component” as used herein refers to a TALEN polypeptide or a nucleic acid (e.g., DNA or RNA) encoding the same.

A “transcription activator-like effector nuclease (TALEN) polypeptide” as used herein is an artificial nuclease which comprises a site-specific transcription activator-like (TAL) effector domain and a nuclease domain. A TALEN polypeptide can be used to edit an endogenous nucleic acid of an enucleated erythroid cell.

As used herein, the term “variant” of a polypeptide refers to a polypeptide having at least one sequence difference compared to that polypeptide, e.g., one or more substitutions, insertions, or deletions. In some embodiments, the variant has at least 70%, 80%, 85%, 90%, 95%, 96%, 97%, 98%, or 99% identity to that polypeptide. A variant includes a fragment. In some embodiments, a fragment lacks up to 1, 2, 3, 4, 5, 10, 20, or 100 amino acids on the N-terminus, C-terminus, or both (each independently), compared to the full-length polypeptide.

A “Zinc Finger (ZF) component” as used herein refers to a ZF polypeptide or a nucleic acid (e.g., DNA or RNA) encoding the same.

A “ZF protein”, “ZF polypeptide” or “Zinc Finger protein” as used herein refers to a protein which comprises a site-specific zinc finger domain. An example of a ZF protein is a ZFN protein. A ZF protein may further comprise a transcriptional activator or repressor domain.

A “Zinc Finger nuclease (ZFN) component” as used herein refers to a ZFN polypeptide or a nucleic acid (e.g., DNA or RNA) encoding the same.

A “ZFN” or “Zinc Finger Nuclease” is artificial nuclease which comprises a site-specific zinc finger domain and a nuclease domain. A ZFN can be used to edit the endogenous gene of an erythroid cell.

### Exemplary Polypeptides and Uses Thereof

An erythroid cell described herein can have increased or decreased levels of an endogenous protein or nucleic acid (e.g., DNA or RNA) described herein. In some embodiments, a nucleated erythroid cell has decreased levels of the endogenous protein, DNA, or RNA as compared to an otherwise similar unmodified erythroid cell. In some embodiments, an enucleated erythroid cell has decreased levels of the endogenous protein or RNA as compared to an otherwise similar unmodified erythroid cell. In some embodiments, one or more of the endogenous proteins or RNA is encoded by a gene selected from Table 1 or Table 2. In some embodiments, the endogenous protein or RNA is a naturally occurring allele or polymorph of a sequence described in Table 1 or Table 2. In some embodiments, the endogenous protein is greater than 1,000, 1,500, 2,000, 2,500, 3,000, 4,000, 5,000, 10,000, 15,000, 20,000, 25,000, or 30,000 amino acids in length. In some embodiments, the endogenous protein is one that is difficult to express as a transgene, e.g., due to its large size.

The erythroid cell may further comprise an exogenous protein. An exemplary exogenous protein, e.g., a protein of Table 3 or 4 or a variant thereof, includes:
a) a naturally occurring form of the protein;
b) the protein having a sequence appearing in a database, e.g., GenBank database, on Dec. 29, 2017;
c) a protein having a sequence that differs by no more than 1, 2, 3, 4, 5 or 10 amino acid residues from a sequence of a) or b);
d) a protein having a sequence that differs at no more than 1, 2, 3, 4, 5 or 10% its amino acids residues from a sequence of a) or b);
e) a protein having a sequence that does not differ substantially from a sequence of a) or b); or
f) a protein having a sequence of c), d), or e) that does not differ substantially in a biological activity, e.g., an enzymatic activity (e.g., specificity or turnover) or binding activity (e.g., binding specificity or affinity) from a protein having the sequence of a) or b).

In some embodiments, the protein comprises a protein or fragment thereof, e.g., all or a fragment of a protein of a), b), c), d), e), or f) of the preceding paragraph. In some embodiments, the protein comprises a protein of Table 3 or 4, or an active protein having an amino acid sequence identity of at least 70%, 75%, 80%, 85%, 90%, 95%, 96%, 97%, 98%, or 99% identity thereto.

In some embodiments, an exogenous polypeptide described herein is at least 200, 300, 400, 500, 600, 700, or 800 amino acids in length. In some embodiments, the exogenous polypeptide is between 200-300, 300-400, 400-500, 500-600, 600-700, or 700-800 amino acids in length.

In some embodiments, an erythroid cell, e.g., an enucleated erythroid cell, comprises at least 1,000, 5,000, 10,000, 15,000, 20,000, 25,000, 30,000, 50,000, 100,000, 200,000, or 500,000 copies of an exogenous polypeptide described herein, e.g., of Table 3 or 4.

In some embodiments, an exogenous polypeptide described herein is a fusion protein, e.g., a fusion with an endogenous red blood cell protein or fragment thereof, e.g., an intracellular protein or a transmembrane protein, e.g., GPA, Kell, CD71, or a transmembrane fragment thereof. In some embodiments, the transmembrane protein is a type-1 transmembrane protein, a type-2 transmembrane protein, or a type-3 transmembrane protein. In some embodiments, the transmembrane protein or fragment thereof has an extracellular N-terminus, and in other embodiments, the transmembrane protein or fragment thereof has an extracellular C-terminus. In some embodiments, one or more of the exogenous polypeptides is not a fusion protein. In some embodiments, one or more of the exogenous polypeptides is not fused to an endogenous erythrocyte protein or fragment thereof.

In some embodiments, an exogenous polypeptide described herein (e.g., a protein comprising an antigen, e.g., an autoimmune antigen) is intracellular.

In some embodiments, the exogenous protein described herein comprises a leader sequence (e.g., a naturally-occurring leader sequence or a synthetic leader sequence). In some embodiments, the exogenous protein lacks a leader sequence (e.g., is genetically modified to remove a naturally-occurring leader sequence). In some embodiments, the exogenous protein comprises an N-terminal methionine residue. In some embodiments, the exogenous protein lacks an N-terminal methionine residue.

While many of the compositions and methods herein involve modulation of endogenous gene expression, it is understood that the disclosure also contemplates erythroid cells comprising an exogenous polypeptide described herein, e.g., an exogenous polypeptide of Table 1. In some embodiments, the erythroid cell has increased or decreased expression of an endogenous gene, and in other embodiments, the erythroid cell does not have increased or decreased expression of any endogenous genes. In some embodiments, the exogenous polypeptide comprises CD58, CR1, CD47, ATP11C, CD55, CD59, Bcl-xL, VKORC1, GGCX, CYB5R3, SOD1, GPX1, GPX4, or PRDX2, or a protein having at least 70%, 75%, 80%, 85%, 90%, 95%, 96%, 97%, 98%, or 99% amino acid sequence identity thereto. In some embodiments, the exogenous polypeptide comprises a phospholipid transportation enzyme or P-IV ATPase family protein, e.g., ATP11C, or a protein having at least 70%, 75%, 80%, 85%, 90%, 95%, 96%, 97%, 98%, or 99% amino acid sequence identity thereto. In some embodiments, the exogenous polypeptide comprises a protein that post-translationally modifies a clotting factor (e.g., VKORC1 or GGCX), or a protein having at least 70%, 75%, 80%, 85%, 90%, 95%, 96%, 97%, 98%, or 99% amino acid sequence identity thereto. In some embodiments, the exogenous polypeptide comprises a protein that increased redox capacity of a cell (e.g., CYB5R3, SOD1, GPX1, GPX4, or PRDX2) or a protein having at least 70%, 75%, 80%, 85%, 90%, 95%, 96%, 97%, 98%, or 99% amino acid sequence identity thereto.

Exemplary Endogenous Polypeptides and Nucleic Acids (e.g., DNA or RNA) that can be Regulated Using the Methods Herein

### CD58 Protein and Nucleic Acid

In some embodiments, the endogenous protein comprises endogenous CD58. In some embodiments, the endogenous nucleic acid (e.g., DNA or RNA) is a CD58-encoding DNA or RNA. In some embodiments, the endogenous CD58 protein is encoded by a nucleic acid comprising the sequence of Genbank Accession No. Y14780.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous CD58-encoding nucleic acid is a naturally occurring allele or polymorph of the sequence of Genbank Accession No. Y14780, or a CD58 protein encoded by the same.

In some embodiments, an erythroid cell described herein has increased CD58 as compared to an otherwise similar unmodified cell. In some embodiments, an increase in a level of endogenous CD58 or CD58-encoding nucleic acid (e.g., DNA or RNA) in an erythroid cell, e.g., an enucleated erythroid cell, results in increased immunogenicity. In some embodiments, the immunogenicity of the erythroid cell, e.g., the enucleated erythroid cell, is increased relative to an unmodified otherwise similar erythroid cell. In some embodiments, an increase in the level of endogenous CD58 or CD58-encoding nucleic acid (e.g., DNA or RNA) in the erythroid cell, e.g., the enucleated erythroid cell, is greater than about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell with an increased level of CD58, has activity selected from one, two, or more (e.g., all) of stimulating T cells (e.g., proliferation of T cells (e.g., CD4⁺ or CD8⁺ T cells), e.g., in the presence of an anti-CD3 antibody), altering T cell phenotype (e.g., decreased level (e.g., expression) or activity of an immune checkpoint inhibitor (e.g., PD-1) or increased level (e.g., expression) or activity of a T cell memory marker, e.g., an interleukin receptor (e.g., IL7R) on the T cell), and stimulating cytokine release (e.g., one, two, three, or more (e.g., all) of IL-2, IL-4, IL-10, and IFNγ by the T cell). In some embodiments, erythroid cells having an increased level of CD58 promote increased proliferation of T cells, e.g., CD8+ T cells (see, e.g., Leitner et al “CD58/CD2 Is the Primary Costimulatory Pathway in Human CD28-CD8+ T Cells” J Immunol 2015, 195 (2) 477-487. In some embodiments, erythroid cells having an increased level of CD58 promote T cells (e.g., CD4+ T cells) to adopt a more regulatory phenotype, e.g., a Tr1 phenotype, e.g., to promote tolerance (see, e.g., Bullens et al. (2001) International Immunology 13(2): 181-191.) PD-1 and IL7R levels can be measured, e.g., using an assay as described in McKinney et al. (2015) Nature 523(7562): 612-616, which is herein incorporated by reference in its entirety. An increase in immunogenicity, e.g., proliferation of T cells, can be measured, e.g., using an assay as described in Wakkach A et al. (2001) J. Immunol. 167,3107-3113, which is herein incorporated by reference in its entirety.

In other embodiments, erythroid cells comprising decreased levels of endogenous CD58 are provided. In some embodiments, a decrease in a level of endogenous CD58 or CD58-encoding nucleic acid (e.g., DNA or RNA) in an erythroid cell, e.g., an enucleated erythroid cell, results in decreased immunogenicity. In some embodiments, the immunogenicity of the erythroid cell, e.g., the enucleated erythroid cell having decreased CD58 levels, is decreased relative to an unmodified otherwise similar erythroid cell. In some embodiments, a decrease in the level of endogenous CD58 or CD58-encoding nucleic acid (e.g., DNA or RNA) in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell with decreased levels of CD58, has activity selected from one, two, or more (e.g., all) of decreased T cell stimulation (e.g., decreased proliferation of T cells (e.g., CD4⁺ or CD8⁺ T cells), e.g., in the presence of an anti-CD3 antibody), altering T cell phenotype (e.g., increased level (e.g., expression) or activity of an immune checkpoint inhibitor (e.g., PD-1) or decreased level (e.g., expression) or activity of a T cell memory marker, e.g., an interleukin receptor (e.g., IL7R)), and decreased cytokine release (e.g., one, two, three, or more (e.g., all) of IL-2, IL-4, IL-10, and IFNγ by the T cell). A decrease in immunogenicity can be measured, e.g., using an assay as described in Wakkach A et al. (2001) J. Immunol. 167: 3107-3113, which is herein incorporated by reference in its entirety.

### CR1 Protein and Nucleic Acid

In some embodiments, the endogenous protein comprises endogenous CR1. In some embodiments, the endogenous nucleic acid (e.g., DNA or RNA) is a CR1-encoding DNA or RNA. In some embodiments, the endogenous CR1 protein is encoded by a nucleic acid comprising the sequence of Genbank Accession No. NP_000564.2, NP_000642.3, or AL137789.11, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous CR1-encoding nucleic acid is a naturally occurring allele or polymorph of the sequence of Genbank Accession No. NP_000564.2, NP_000642.3, or AL137789.11, or a CR1 protein encoded by the same.

In some embodiments, an erythroid cell described herein has increased CR1 as compared to an otherwise similar unmodified cell. In some embodiments, an increase in a level of endogenous CR1 or CR1-encoding nucleic acid (e.g., DNA or RNA) in an erythroid cell, e.g., an enucleated erythroid cell, results in one or both of binding to immune complexes and clearing immune complexes from circulation, e.g., to treat one or both of an autoimmune disease or an infectious disease. In some embodiments, the one or both of binding immune complexes and clearing the immune complexes from circulation is increased relative to an unmodified otherwise similar erythroid cell. In some embodiments, an increase in the level of endogenous CR1 or CR1-encoding nucleic acid (e.g., DNA or RNA) in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., the enucleated erythroid cell, has activity of one, two, or more (e.g., all) of binding immune complexes, delivery of the immune complexes to macrophages (e.g., in the liver and spleen), and phagocytosis of the immune bound to the erythroid cells by macrophages. Immune complex binding of CR1-expressing cells and immune complex transfer to macrophages can be measured, e.g., using an assay described in Example 14 of International Application WO2015/073587, which is herein incorporated by reference in its entirety.

### CD47 Protein and Nucleic Acid

CD47 is a membrane protein. In some embodiments, CD47 interacts with the myeloid inhibitory immunoreceptor SIRPα (also termed CD172a or SHPS-1) that is present, e.g., on macrophages. In some embodiments, engagement of SIRPα by CD47 provides a down-regulatory signal that inhibits host cell phagocytosis. For example, high levels of CD47 allow cancer cells to avoid phagocytosis despite the presence pro-phagocytic signals, such as high levels of calreticulin. CD47 also has further roles in cell adhesion, e.g., by acting as an adhesion receptor for THBS1 on platelets and in the modulation of integrins. In some embodiments, CD47 interaction with SIRPα further prevents maturation of immature dendritic cells, inhibits cytokine production by mature dendritic cells. CD47 interaction with SIRPγ mediates cell-cell adhesion, enhances superantigen-dependent T-cell-mediated proliferation and co-stimulates T-cell activation.

CD47 is typically a 50 kDa membrane receptor that has an extracellular N-terminal IgV domain, five transmembrane domains, and a short C-terminal intracellular tail. There are four alternatively spliced isoforms of CD47 that differ only in the length of their cytoplasmic tail. The cytoplasmic tail can be found as four different splice isoforms ranging from 4 to 36 amino acids. The 16 amino acid form 2 is typically expressed in all cells of hematopoietic origin and in endothelial and epithelial cells. The 36 amino acid form 4 is expressed primarily in neurons, intestine, and testis. The 4 amino acid form 1 is found in epithelial and endothelial cells. The expression pattern of the 23 amino acid form 3 resembles that of form 4.

In some embodiments, the endogenous protein comprises endogenous CD47. In some embodiments, the endogenous nucleic acid (e.g., DNA or RNA) is a CD47-encoding DNA or RNA. In some embodiments, the endogenous CD47 protein is encoded by a nucleic acid comprising the sequence of Genbank Accession No. AK289813.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous CD47-encoding nucleic acid is a naturally occurring allele or polymorph of the sequence of Genbank Accession No. AK289813.1, or a CD47 protein encoded by the same.

In some embodiments, an erythroid cell described herein has reduced CD47 compared to an otherwise similar unmodified cell. In some embodiments, a decrease in a level of endogenous CD47 or CD47-encoding nucleic acid (e.g., DNA or RNA) in an erythroid cell, e.g., an enucleated erythroid cell, results in decreased circulation time after administration to a subject, as compared to an unmodified otherwise similar erythroid cell. In some embodiments, a decrease in a level of endogenous CD47 or CD47-encoding nucleic acid (e.g., DNA or RNA) in an erythroid cell, e.g., an enucleated erythroid cell, results in increased engulfment by macrophages, as compared to an unmodified otherwise similar erythroid cell. In some embodiments, a decrease in the level of endogenous CD47 or CD47-encoding nucleic acid (e.g., DNA or RNA) in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, it can be useful to decrease CD47 levels. In some embodiments, a decrease in a level of endogenous CD47 or CD47-encoding nucleic acid (e.g., DNA or RNA) in an erythroid cell, e.g., an enucleated erythroid cell, results in one or both of decreased circulation time and decreased tolerogenic potential, e.g., using signals that negatively regulate phagocytes. In some embodiments, one or both of circulation time and tolerogenic potential of the erythroid cell, e.g., the enucleated erythroid cell, is decreased relative to the circulation time and tolerogenic potential of an unmodified otherwise similar erythroid cell. In some embodiments, a decrease in the level of endogenous CD47 or CD47-encoding nucleic acid (e.g., DNA or RNA) in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

Accordingly, the disclosure provides, for instance, an erythroid cell e.g., enucleated erythroid cell, lacking an endogenous CD47 or CD47-encoding nucleic acid (e.g., DNA or RNA), or having the endogenous CD47 or CD47-encoding nucleic acid (e.g., DNA or RNA) present at a level less than about 50%, 40%, 30%, 20%, 10%, 5%, 2%, or 1% compared to an unmodified otherwise similar erythroid cell. Similarly, in some embodiments, the disclosure provides an erythroid cell lacking an endogenous CD47 or CD47-encoding nucleic acid (e.g., DNA or RNA), or having the endogenous CD47 or CD47-encoding nucleic acid (e.g., DNA or RNA) present at a level less than about 50%, 40%, 30%, 20%, 10%, 5%, 2%, or 1% compared to an unmodified otherwise similar erythroid cell.

In some embodiments, the erythroid cell is capable of exhibiting increased adherence to a macrophage, increased engulfment by a macrophage, or both of increased adherence to and increased engulfment by a macrophage, e.g., a non-LPS-stimulated macrophage or an LPS-stimulated macrophage, e.g., in a flow cytometry assay, e.g., an assay of Example 4. In some embodiments, the erythroid cell further comprises an exogenous polypeptide, e.g., an antigen, e.g., an autoimmune antigen. In some embodiments, the antigen is intracellular. In some embodiments, the antigen is present at the surface of the erythroid cell, e.g., wherein the exogenous antigen is fused to a transmembrane domain.

In some embodiments (e.g., embodiments involving a plurality of erythroid cells lacking CD47 or having reduced levels of CD47), the population of cells exhibits increased adherence to, engulfment by, or both of adherence to and engulfment by macrophages as compared to a population of otherwise similar unmodified erythroid cells. In some embodiments, the number of cells in the population that adhere to, are engulfed by, or both of adhere to and are engulfed by macrophages is at least about 105%, 110%, 113%, 115%, 120%, 125%, 130%, 140%, or more of the number of cells in the population of otherwise similar unmodified erythroid cells that adhere to, are engulfed by, or both adhere and are engulfed, by similar macrophages, e.g., in an assay of Example 4. In some embodiments, at least 80%, 85%, 90%, 95%, 96%, 97%, 97.5%, 98%, 99%, 99.2%, or 99.5% of the enucleated erythroid cells in the population adhere to macrophages (e.g., LPS-stimulated macrophages), e.g., in an assay of Example 4. In some embodiments, less than 50%, 45%, 41.6%, 40%, 35%, 30%, 28.1%, 25%, 20%, 15%, 10%, 5%, 1%, or less of cells in the population are CD47-positive, e.g., as measured in an assay of Example 3.

In some embodiments, upon administration to a subject, the enucleated erythroid cells in the population have a half-life which is less than 90%, 80%, 70%, 60%, 50%, 40%, 30%, 20%, or 10% of the half-life of a population of otherwise similar unmodified enucleated erythroid cells.

While not wishing to be bound by theory, in some embodiments, knocking out CD47 in a nucleated erythroid cell precursor abrogates the production of mRNA encoding CD47. Over subsequent cell divisions, mRNA encoding CD47 and CD47 polypeptide are subject to the cellular degradation machinery, and/or are diluted during cellular division. In some embodiments, upon enucleation, only trace amounts of CD47 remain due to these degradation and/or dilution processes. In some embodiments, a population of erythroid cells comprises CD47− cells and CD47+ cells. In some embodiments, less than 50%, 45%, 41.6%, 40%, 35%, 30%, 28.1%, 25%, 20%, 15%, 10%, or 5% of cells in the population have wild-type CD47 levels, e.g., are CD47-positive in an assay of Example 3. The proportion of CD47-negative cells in a population can be measured, e.g., using one or more (e.g., all) of the following steps: i) staining the cells with fluorescein isothiocyanate (FITC)-conjugated anti-human CD47 antibody (BIOLEGEND), and ii) performing fluorescence-activated cell sorting (FACS) to detect FITC levels. In some embodiments, the CD47− enucleated erythroid cell is descended from (i.e., the progeny of) an erythroid progenitor cell that was null for the CD47 gene.

In some embodiments, a cell population comprising CD47-positive erythroid cells and CD47-negative erythroid cells can be enriched for a desired cell type, e.g., CD47-negative erythroid cells. For instance, the cell population can be contacted with an agent that binds CD47 (e.g., an anti-CD47 antibody, e.g., an antibody comprising a fluorescent or magnetic label) under conditions that allow binding of the antibody to cells comprising CD47. The cells can then be sorted (e.g., using FACS or MACS) to enrich for CD47-negative cells, or cells comprising CD47 can be removed from the population (e.g., by mechanical means). In some embodiments, a population of nucleated erythroid cells is enriched to yield a population of nucleated erythroid cells enriched for CD47− negative cells; the nucleated CD47− negative population can then be differentiated to yield enucleated CD47− negative cells. In some embodiments, a population of enucleated erythroid cells is enriched to yield a population of enucleated erythroid cells enriched for CD47− negative cells. In some embodiments, the enriched population comprises less than 50%, 45%, 41.6%, 40%, 35%, 30%, 28.1%, 25%, 20%, 15%, 10%, or 5% CD47-positive cells. In some embodiments, the enriched cell population comprises at least 60%, 65%, 70%, 75%, 80%, 85%, 90%, or 95% CD47-negative cells.

In some embodiments, a population of enucleated erythroid cells having reduced CD47 levels (e.g., substantially lacking CD47), or lacking CD47, exhibit increased adherence and/or engulfment by macrophages. Adherence and/or engulfment of a CD47− cell can be assayed, e.g., using a flow cytometry, e.g., as described in Example 4. For example, the assay can comprise one or more of, e.g., all of: i) labeling enucleated erythroid cells with CellTrace™ Far Red (CTFR) (THERMOFISHER) according to manufacturer's instructions, ii) admixing 2×10⁵ lipopolysaccharide (LPS)-stimulated or 2×10⁵ non-stimulated adherently-cultured macrophages with either 2×10⁶ CD47 knockout enucleated erythroid cells or 2×10⁶ control enucleated erythroid cells, and iii) incubated the admixed cells for 16 hours at 37° C., iv) removing supernatant, v) washing cells with 500 μL phosphate buffered saline (PBS), vi) detaching cells from the culture plates, vii) staining macrophages with Pacific Blue-labelled anti-CD14 antibody (BIOLEGEND), viii) counting CD14-positive/CTFR-positive cells as macrophages that were adhered to or had engulfed an erythroid cell, and ix) counting CD14-negative/CTFR-positive cells as erythroid cells that had not been adhered to or engulfed.

In some embodiments, in a population of enucleated erythroid cells having reduced CD47 levels (e.g., substantially lacking CD47), at least 80%, 85%, 90%, 95%, 96%, 97%, 97.5%, 98%, 99%, 99.2%, or 99.5% of the enucleated erythroid cells adhere to or are engulfed by macrophages, e.g., in the assay described above. In some embodiments, number of enucleated erythroid cells having reduced CD47 levels (e.g., CD47− cells) that adhere to or are engulfed by macrophages is at least 100%, 105%, 100%, 113%, 115%, 120%, 125%, 130%, or 140% of the number of otherwise similar unmodified enucleated erythroid cells (e.g., CD47+ cells) engulfed by macrophages, e.g., in the assay described above.

In some embodiments, the erythroid cell, e.g., an enucleated erythroid cell, with reduced CD47 levels has activity of one or both of increasing phagocytosis, e.g., by macrophages (e.g., liver macrophages) or the THP-1 monocytic cell line, or decreasing persistence, e.g., half-life, in circulation. Increased phagocytosis by macrophages or THP-1 cells can be measured, e.g., using an assay as described in Burger, P et al. (2012) Blood 119(23): 5512-5521,” which is herein incorporated by reference in its entirety.

In some embodiments, one or more of (e.g., all of) CD47 form 1, from 2, form 3, or form 4 of CD47 (or the RNA encoding these forms of CD47) is absent from the erythroid cell, or is present at a lower level than an otherwise similar wild-type erythroid cell.

In some embodiments, the level of CD47 in the erythroid cell (e.g., enucleated erythroid cell) is such that the erythroid cell resides in circulation after administration to a subject for less than about 15 days, about 21 days, about 30 days, about 45 days, about 60 days, about 100 days, or about 120 days. In some embodiments, the level of CD47 in a plurality of erythroid cells (e.g., enucleated erythroid cells) is such that the plurality of erythroid cells has a half-life in circulation for less than 15 days, 21 days, 30 days, 45 days, 60 days, 100 days, 120 days.

In some embodiments, an erythroid cell described herein has increased CD47 as compared to an otherwise similar unmodified cell. In some embodiments, an increase in a level of endogenous CD47 or CD47-encoding RNA in an erythroid cell, e.g., an enucleated erythroid cell, results in one or both of increased circulation time after administration to a subject and/or increased tolerogenic potential, e.g., using signals that negatively regulate phagocytes (sometimes called a “don't eat me” signal). In some embodiments, one or both of circulation time and tolerogenic potential of the erythroid cell, e.g., the enucleated erythroid cell, is increased relative to the circulation time and tolerogenic potential of an unmodified otherwise similar erythroid cell. In some embodiments, an increase in the level of endogenous CD47 or CD47-encoding nucleic acid (e.g., DNA or RNA) in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

### Exogenous Antigens

In some embodiments, an erythroid cell described herein, e.g., an erythroid cell having decreased levels of CD47, or lacking CD47, further comprises an antigen. In some embodiments, the cell is made by a process involving introducing the protein antigen directly into the cell, e.g., using hypotonic loading. In some embodiments, the cell is made by a process comprising introducing a nucleic acid encoding the antigen into the cell or a precursor thereof, under conditions that allow for expression of the antigen. The antigen can be, for example, a protein of Table 4 herein, or an antigenic fragment thereof. In some embodiments, the antigenic fragment has a length of 10-20, 20-30, 30-40, 40-50, or 50-100 amino acids. In some embodiments, the protein comprising the antigen has no more than 1, 2, 3, 4, 5, or 10 amino acid differences relative to the corresponding wild-type protein of Table 4. In some embodiments, the antigenic fragment has no more than 1, 2, 3, 4, 5, or 10 amino acid differences relative to the corresponding region of the corresponding wild-type protein of Table 4. In some embodiments, the antigen is part of a fusion or chimeric protein, e.g., a fusion protein that comprises a transmembrane domain. In some embodiments, the antigen is present at a copy number of at least 100, 200, 500, 1,000, 2,000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 9000,000, or 1,000,000 copies per cell, or in an amount of 100-200, 200-500, 500-1,000, 1,000-2,000, 2,000-5,000, 5,000-10,000, 10,000-20,000, 20,000-30,000, 30,000-40,000, 40,000-50,000, 50,000-100,000, 100,000-200,000, 200,000-500,000, or 500,000-1,000,000 copies per cell.

In some embodiments, an enucleated erythroid cell (e.g., a CD47-negative cell) comprising an antigen of Table 4 is used to treat an autoimmune disease of Table 4.

While not wishing to be limited to any particular mechanism, it is believed that peripheral immunological tolerance can be induced by self-antigens from apoptosing cells (Griffith and Ferguson, Immunity 2011; Green et al., Nat Rev Immunol 2009). Therapeutic strategies that harness the tolerogenic potential of apoptosing cells to induce peripheral immune tolerance are under investigation. These strategies typically involve the chemical coupling of antigens of interest to the surface of cells. In studies in mice, rat, and guinea pigs, a variety of protein antigens are chemically attached to the surface of splenocytes and leukocytes. See, e.g., Miller et al., J Exp Med 1979; Braley-Mullen et al., Cell Immunol 1980; Luo et al., PNAS 2008; Smarr et al., J Immunol 2011.

A large number of erythrocytes are cleared after apoptosis-like programmed cell death, called eryptosis, each day (more than 1% per day in humans, approximately 1×10¹¹ cells). Although the exact triggers of erythrocyte clearance remain unclear, eryptotic erythrocytes are characterized by phosphatidylserine asymmetry, membrane heterogeneity, and annexin-V binding, analogous to apoptotic nucleated cells.

According to the non-limiting theory herein, an enucleated erythroid cell comprising an exogenous antigen can be engulfed by a macrophage, allowing the macrophage to display the antigen on its cell surface, thereby inducing tolerance to the antigen. As shown in working Example 3 herein, reducing CD47 expression in enucleated erythroid cells increases erythroid cell adherence to and/or engulfment by macrophages. Thus, erythroid cells with reduced CD47 expression that comprise an exogenous antigen are expected to increase the amount of antigen delivered to macrophages, thereby increasing the induction of immune tolerance to the antigen.

### ATP11C (Flippase) Protein and Nucleic Acid

In some embodiments, an erythroid cell described herein has increased ATP11C compared to an otherwise similar unmodified cell. In some embodiments, the endogenous protein comprises endogenous phospholipid transportation enzyme or P-IV ATPase family protein, e.g., ATP11C (flippase). In some embodiments, the endogenous nucleic acid (e.g., DNA or RNA) encodes a phospholipid transportation enzymes or P-IV ATPase family protein, e.g., is an ATP11C-encoding RNA. In some embodiments, the endogenous ATP11C protein is encoded by a nucleic acid comprising the sequence of Genbank Accession No. AL161777.4 or NM_173694.4, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous ATP11C -encoding nucleic acid is a naturally occurring allele or polymorph of the sequence of Genbank Accession No. AL161777.4 or NM_173694.4, or an ATP11C protein encoded by the same.

In some embodiments, an increase in a level of endogenous ATP11C or ATP11C-encoding nucleic acid (e.g., DNA or RNA) in an erythroid cell, e.g., an enucleated erythroid cell, results in one or both of increased internalization of phosphatidylserine (PS) to the inner membrane leaflet, decreased PS surface exposure, or increased circulation time after administration to a subject. In some embodiments, circulation time is determined by measuring for how much time after administration a cell is detectable in the subject. In some embodiments, one or both of internalization of PS to the inner membrane and circulation time of the erythroid cell, e.g., the enucleated erythroid cell, is increased relative to an unmodified otherwise similar erythroid cell. In some embodiments, PS surface exposure in the membrane of the enucleated erythroid cell, is decreased relative to an unmodified otherwise similar erythroid cell. In some embodiments, an increase in the level of endogenous ATP11C or ATP11C-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of internalizing PS to the inner membrane leaflet and increased circulation time. Internalization of PS to the inner membrane leaflet can be measured, e.g., using a fluorescent PS (NBD-PS) internalization assay, e.g., by an assay as described in Arashiki et al. (2016), Haematologica 101(5): 559-565, which is herein incorporated by reference in its entirety. Surface PS levels can be measured, e.g., by Ca²⁺-dependent binding of fluorescently labeled annexin V, e.g., as described in Arashiki et al. supra. The circulation time can be measured, e.g., by in vivo life span measurements using carboxyfluorescein diacetate succinimidyl ester (CFSE) in conjunction with mathematical modeling, e.g., using an assay as described in Yabas et al. (2014). J Biol. Chem., 289(28): 19531-19537, which is herein incorporated by reference in its entirety.

### PLSCR1 (Scramblase) Protein and Nucleic Acid

In some embodiments, an erythroid cell described herein has decreased Scramblase compared to an otherwise similar unmodified cell. In some embodiments, the endogenous protein comprises endogenous PLSCR1 (Scramblase). In some embodiments, the endogenous nucleic acid (e.g., DNA or RNA) is a PLSCR1-encoding DNA or RNA. In some embodiments, the endogenous PLSCR1 protein is encoded by a nucleic acid comprising the sequence of Genbank Accession No. AK300181.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous PLSCR1-encoding nucleic acid is a naturally occurring allele or polymorph of the sequence of Genbank Accession No. AK300181.1, or a PLSCR1 protein encoded by the same.

In some embodiments, a decrease in a level of endogenous PLSCR1 or PLSCR1-encoding nucleic acid (e.g., DNA or RNA) in an erythroid cell, e.g., an enucleated erythroid cell, results in one or both of decreased phosphatidylserine (PS) surface exposure in the membrane or increased circulation time. In some embodiments, PS surface exposure in the membrane of the enucleated erythroid cell, is decreased relative to an unmodified otherwise similar erythroid cell. In some embodiments, circulation time of the erythroid cell, e.g., the enucleated erythroid cell, is increased relative to the circulation time of an unmodified otherwise similar erythroid cell. In some embodiments, a decrease in the level of endogenous PLSCR1 or PLSCR1-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of decreased surface exposure of PS to the outer membrane leaflet and increased circulation. Surface PS levels can be measured, e.g., by Ca²⁺-dependent binding of fluorescently labeled annexin V, e.g., as described in Arashiki et al. supra. The circulation time can be measured, e.g., by an assay as described in Yabas, M et al. supra.

### CD55 Protein and Nucleic Acid

In some embodiments, an erythroid cell described herein has increased CD55 compared to an otherwise similar unmodified cell. In some embodiments, the endogenous protein comprises endogenous CD55 (decay-accelerating factor). In some embodiments, the endogenous nucleic acid (e.g., DNA or RNA) is a CD55-encoding DNA or RNA. In some embodiments, the endogenous CD55 protein is encoded by a nucleic acid comprising the sequence of Genbank Accession No. AB240567.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous CD55-encoding nucleic acid is a naturally occurring allele or polymorph of the sequence of Genbank Accession No. AB240567.1, or a CD55 protein encoded by the same.

In some embodiments, an increase in a level of endogenous CD55 or CD55-encoding nucleic acid (e.g., DNA or RNA) in an erythroid cell, e.g., an enucleated erythroid cell, results in one or both of increased complement inhibition or increased circulation. In some embodiments, one or both of complement inhibition and circulation of the erythroid cell, e.g., the enucleated erythroid cell, is increased relative to an unmodified otherwise similar erythroid cell. In some embodiments, an increase in the level of endogenous CD55 or CD55-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one, two, three, or more (e.g., all) of decreased susceptibility to hemolysis, decreased C3 deposition, decreased activation of the alternative complement pathway (APC), and increased persistence. Hemolysis of enucleated erythroid cells comprising CD55 can be measured, e.g., by measuring free hemoglobin after incubation in acidified serum, e.g., as described in Wilcox et al. (1991) Blood 78(3): 820-829, which is herein incorporated by reference in its entirety. Activation of the APC and deposition of activated C3 on the membrane surface of the enucleated erythroid cell can be measured, e.g., by assays of zymosan activation and C3 fixation as described in Xiujun Sun et al. (1999) Proc. Nat'l. Acad. Sci. USA 96: 628-633. Persistence of the enucleated erythroid cell can be determined, e.g., as described herein.

### CD59 (MEMBRANE Inhibitor of Reactive Lysis (MIRL)) Protein and Nucleic Acid

In some embodiments, an erythroid cell described herein has increased CD59 compared to an otherwise similar unmodified cell. In some embodiments, the endogenous protein comprises endogenous CD59 (Membrane inhibitor of reactive lysis (MIRL)). In some embodiments, the endogenous CD59 protein is encoded by a nucleic acid comprising the sequence of Genbank Accession No. X17198.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous MIRL-encoding nucleic acid is a naturally occurring allele or polymorph of the sequence of Genbank Accession No. X17198.1, or a MIRL protein encoded by the same.

In some embodiments, an increase in a level of endogenous CD59 or CD59-encoding nucleic acid (e.g., DNA or RNA) in an erythroid cell, e.g., an enucleated erythroid cell, results in one or both of increased complement inhibition or increased circulation time after administration to a subject. In some embodiments, one or both of complement inhibition and circulation of the erythroid cell, e.g., the enucleated erythroid cell, is increased relative to an unmodified otherwise similar erythroid cell. In some embodiments, an increase in the level of endogenous CD59 or CD59-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one, two, three, or more (e.g., all) of decreased susceptibility to hemolysis, decreased C3 deposition, decreased activation of the alternative complement pathway (APC), and increased persistence in circulation, after administration to a subject. Hemolysis of enucleated erythroid cells comprising CD59 can be measured, e.g., by measuring free hemoglobin after incubation in acidified serum, e.g., as described in Wilcox et al. supra. Activation of the APC and deposition of activated C3 on the membrane surface of the enucleated erythroid cell can be measured, e.g., by assays of zymosan activation and C3 fixation as described in Xiujun Sun et al. supra. Persistence of the enucleated erythroid cell can be determined, e.g., as described herein.

### Bcl-xL Protein and Nucleic Acid

In some embodiments, an erythroid cell described herein has increased Bcl-xL compared to an otherwise similar unmodified cell. In some embodiments, the endogenous protein comprises a pro-apoptotic protein e.g., endogenous Bcl-xL. In some embodiments, the endogenous nucleic acid (e.g., DNA or RNA) is a Bcl-xL-encoding DNA or RNA. In some embodiments, the endogenous Bcl-xL protein is encoded by a nucleic acid comprising the sequence of Genbank Accession No. CR936637.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous Bc1-xL-encoding nucleic acid is a naturally occurring allele or polymorph of the sequence of Genbank Accession No. CR936637.1, or a Bcl-xL protein encoded by the same.

In some embodiments, an increase in a level of endogenous Bcl-xL or Bcl-xL-encoding nucleic acid (e.g., DNA or RNA) in an erythroid cell, e.g., an enucleated erythroid cell, results in increased cell survival, e.g., during one or both of erythropoiesis and/or during manufacturing (e.g., cell culture, expansion and differentiation in vitro). In some embodiments, the survival of the erythroid cell, e.g., the enucleated erythroid cell, is increased relative to an unmodified otherwise similar erythroid cell. In some embodiments, an increase in the level of endogenous Bc1-xL or Bc1-xL-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased survival during erythropoiesis after administration to a subject or increased survival during manufacturing (e.g., cell culture, expansion and differentiation in vitro). Survival of the enucleated erythroid cell comprising Bcl-xL can be measured, e.g., using an assay as described in Koulinis (2012) Blood 119(5): 1228-39, which is herein incorporated by reference in its entirety. Fold expansion and cell yield can be measured following ex vivo differentiation of CD34⁺ progenitor cells to enucleated erythroid cells.

### Vitamin K Epoxide Reductase Complex (VKORC1) Protein and Nucleic Acid

In some embodiments, an erythroid cell described herein has increased VKORC1 compared to an otherwise similar unmodified cell. In some embodiments, the endogenous protein comprises endogenous Vitamin K Epoxide Reductase Complex (VKORC1). In some embodiments, the endogenous nucleic acid (e.g., DNA or RNA) is a VKORC1-encoding DNA or RNA. In some embodiments, the endogenous VKORC1 protein is encoded by a nucleic acid comprising the sequence of Genbank Accession No. AY466113.1, or a sequence with at least 70%, 75%, 80%, 85%, 90%, 95%, 96%, 97%, 98%, or 99% identity thereto, or a sequence with no more than 10, 9, 8, 7, 6, 5, 4, 3, 2, or 1 nucleic acid alterations relative thereto, e.g., substitutions, insertions, or deletions. In some embodiments, the endogenous VKORC1-encoding nucleic acid is a naturally occurring allele or polymorph of the sequence of Genbank Accession No. AY466113.1, or a VKORC1 protein encoded by the same.

In some embodiments, an increase in a level of endogenous VKORC1 or VKORC1-encoding nucleic acid (e.g., DNA or RNA) in an erythroid cell, e.g., an enucleated erythroid cell, results in one or both of increased production of active coagulation factors (e.g., factor IX) or post-translation modification of coagulation factors to include γ-carboxyglutamic acid (Gla). In some embodiments, one or both of the production of coagulation factors or post-translational modification of coagulation factors to include Gla in the erythroid cell, e.g., the enucleated erythroid cell, is increased relative to an unmodified otherwise similar erythroid cell. In some embodiments, an increase in the level of endogenous VKORC1 or VKORC1-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased production of active coagulation factors or post-translation modification of coagulation factors to include Gla. Production of active coagulation factors or post-translation modification of coagulation factors to include Gla can be measured, e.g., using an assay as described in Wajih (2005) J. Biol. Chem. 280: 31603-31607, which is herein incorporated by reference in its entirety. Expression of coagulation factor can be measured in the enucleated erythroid cell comprising VKORC1 (e.g., by Western blot or FACS). Gla domain carboxylation can be measured with an anti-Gla antibody. Coagulation factor function can be assessed using a thrombin generation assay or a fluorometric factor specific assay, as described in Hansson and Stenflo (2005) Journal of Thrombosis and Haemostasis 3: 2633-2648, which is herein incorporated by reference in its entirety.

### γ-Carboxylase (GGCX) Protein and Nucleic Acid

In some embodiments, an erythroid cell described herein has increased GGCX compared to an otherwise similar unmodified cell. In some embodiments, the endogenous protein comprises endogenous γ-carboxylase (GGCX). In some embodiments, the endogenous nucleic acid (e.g., DNA or RNA) is a GGCX-encoding DNA or RNA. In some embodiments, the endogenous GGCX protein is encoded by a nucleic acid comprising the sequence of Genbank Accession No. AK297397.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous GGCX -encoding nucleic acid is a naturally occurring allele or polymorph of the sequence of Genbank Accession No. AY466113.1, or a GGCX protein encoded by the same.

In some embodiments, an increase in a level of endogenous GGCX or GGCX-encoding nucleic acid (e.g., DNA or RNA) in an erythroid cell, e.g., an enucleated erythroid cell, results in one or both of increased production of active coagulation factors or post-translation modification of coagulation factors to include γ-carboxyglutamic acid (Gla). In some embodiments, one or both of the production of active coagulation factors or post-translation modification of coagulation factors to include Gla in the erythroid cell, e.g., the enucleated erythroid cell, is increased relative to an unmodified otherwise similar erythroid cell. In some embodiments, an increase in the level of endogenous GGCX or GGCX-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased production of active coagulation factors or post-translation modification of coagulation factors to include Gla. Coagulation factor function can be assessed using a thrombin generation assay or a fluorometric factor specific assay, as described in Hansson, K. and Stenflo, J. (2005), Post-translational modifications in proteins involved in blood coagulation. Journal of Thrombosis and Haemostasis, 3: 2633-264, which is herein incorporated by reference in its entirety.

### NADH-cytochrome b5 Reductase (CYB5R3) Protein and Nucleic Acid

In some embodiments, an erythroid cell described herein has increased CYB5R3 compared to an otherwise similar unmodified cell. In some embodiments, the endogenous protein comprises endogenous NADH-cytochrome b5 reductase (CYB5R3). In some embodiments, the endogenous nucleic acid (e.g., DNA or RNA) is a CYB5R3-encoding DNA or RNA. In some embodiments, the endogenous CYB5R3 protein is encoded by a nucleic acid comprising the sequence of Genbank Accession No. AJ010116.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous CYB5R3-encoding nucleic acid is a naturally occurring allele or polymorph of the sequence of Genbank Accession No. AJ010116.1, or a CYB5R3 protein encoded by the same.

In some embodiments, an increase in a level of endogenous CYB5R3 or CYB5R3-encoding nucleic acid (e.g., DNA or RNA) in an erythroid cell, e.g., an enucleated erythroid cell, results in one or both of increased redox capacity or increased reduction of methemoglobin to hemoglobin. In some embodiments, an increase in a level of endogenous CYB5R3 or CYB5R3-encoding RNA results in improved storage, reduced aging, or improved circulation time after administration to a subject, of the erythroid cell, e.g., the enucleated erythroid cell. In some embodiments, one or both of redox capacity and reduction of methemoglobin to hemoglobin, in the erythroid cell, e.g., the enucleated erythroid cell, is increased relative to an unmodified otherwise similar erythroid cell. In some embodiments, an increase in the level of endogenous CYB5R3 or CYB5R3-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell. In some embodiments, an erythroid cell having increased expression of CYBR3 has improved storage characteristics. In some embodiments, an improved storage characteristic comprises one or more of increased stability when refrigerated (e.g., at 4° C., 3-5° C., 2-6° C., or 1-7° C.). In some embodiments, reduced oxidation of the erythroid cells occurs during storage. In some embodiments, the redox state of the cells is substantially unchanged after refrigeration as compared to the redox state before refrigeration, or is within 5%, 10%, or 20% greater of lower than the pre-refrigeration state, e.g., by an assay of Nagababu et al. or Uchiyama et al. described herein. In some embodiments, an erythroid cell having increased expression of CYBR3 has an increased circulation time or half-life in vivo (e.g., after administration to a subject).

In some embodiments, the erythroid cell, e.g., an enucleated erythroid cell, has activity of one or both of increased redox capacity or increased reduction of methemoglobin to hemoglobin. Measuring the redox state of cells can comprise measuring fluorescent degradation products after treatment with H₂O₂ in engineered erythroid cells vs unmodified erythroid cells, e.g., using a method described in Nagababu et al. (2003) Biochimica et Biophysica Acta 1620(1-3): 211-217, which is incorporated herein by reference in its entirety. Measuring the redox state of cells can comprise measuring the lipid peroxidation of erythroid cells using a thiobarbituric acid test, e.g., as described in Uchiyama et al. (1978) Analytical Biochemistry 86(1): 271-278, which is incorporated herein by reference in its entirety.

### Superoxide Dismutase (SOD1) Protein and Nucleic Acid

In some embodiments, an erythroid cell described herein has increased SOD1 compared to an otherwise similar unmodified cell. In some embodiments, the endogenous protein comprises endogenous superoxide dismutase (SOD1). In some embodiments, the endogenous nucleic acid (e.g., DNA or RNA) is a SOD1-encoding DNA or RNA. In some embodiments, the endogenous SOD1 protein is encoded by a nucleic acid comprising the sequence of Genbank Accession No. X01780.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous SOD1-encoding nucleic acid is a naturally occurring allele or polymorph of the sequence of Genbank Accession No. X01780.1, or a SOD1 protein encoded by the same.

In some embodiments, an increase in a level of endogenous SOD1 or SOD1-encoding RNA in an erythroid cell, e.g., an enucleated erythroid cell, results in one or both of increased redox capacity or increased scavenging of superoxide anions. In some embodiments, an increase in a level of endogenous SOD1 or SOD1-encoding RNA results in improved storage, aging, or circulation time of the erythroid cell. In some embodiments, one or both of redox capacity or scavenging of superoxide anions in the erythroid cell, e.g., the enucleated erythroid cell, is increased relative to an unmodified otherwise similar erythroid cell. In some embodiments, an increase in the level of endogenous SOD1 or SOD1-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased redox capacity or increased scavenging of superoxide anions. Assays for determining the redox state of cells are described in Nagababu et al. supra and Uchiyama et al. supra.

### Peroxiredoxin 2 (PRDX2) Protein and Nucleic Acid

In some embodiments, an erythroid cell described herein has increased PRDX2 compared to an otherwise similar unmodified cell. In some embodiments, the endogenous protein comprises endogenous peroxiredoxin 2 (PRDX2). In some embodiments, the endogenous nucleic acid (e.g., DNA or RNA) is a PRDX2-encoding DNA or RNA. In some embodiments, the endogenous PRDX2 protein is encoded by a nucleic acid comprising the sequence of Genbank Accession No. CR541789.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous PRDX2-encoding nucleic acid is a naturally occurring allele or polymorph of the sequence of Genbank Accession No. CR541789.1, or a PRDX2 protein encoded by the same.

In some embodiments, an increase in a level of endogenous PRDX2 or PRDX2-encoding nucleic acid (e.g., DNA or RNA) in an erythroid cell, e.g., an enucleated erythroid cell, results in one or both increased redox capacity or increased scavenging of peroxides and/or peroxynitrite (PN). In some embodiments, an increase in a level of endogenous PRDX2 or PRDX2-encoding nucleic acid (e.g., DNA or RNA) results in improved storage, aging, or circulation time of the erythroid cell. In some embodiments, one or both of redox capacity or scavenging of peroxides and/or PN in the erythroid cell, e.g., the enucleated erythroid cell, is increased relative to an unmodified otherwise similar erythroid cell. In some embodiments, an increase in the level of endogenous PRDX2 or PRDX2-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased redox capacity or scavenging of peroxides and PN. Assays for determining the redox state of cells are described in Nagababu et al. supra and Uchiyama et al. supra.

### Glutathione Peroxidase 1 (GPX1) Protein and Nucleic Acid

In some embodiments, an erythroid cell described herein has increased GPX1 compared to an otherwise similar unmodified cell. In some embodiments, the endogenous protein comprises endogenous glutathione peroxidase 4 (GPX1). In some embodiments, the endogenous nucleic acid (e.g., DNA or RNA) is a GPX1-encoding nucleic acid (e.g., DNA or RNA). In some embodiments, the endogenous GPX1 protein is encoded by a nucleic acid comprising the sequence of Genbank Accession No. X13710.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous GPX1-encoding nucleic acid is a naturally occurring allele or polymorph of the sequence of Genbank Accession No. X13710.1, or a GPX1protein encoded by the same.

In some embodiments, an increase in a level of endogenous GPX1 or GPX1-encoding nucleic acid (e.g., DNA or RNA) in an erythroid cell, e.g., an enucleated erythroid cell, results in one or both increased redox capacity or increased scavenging of peroxides and peroxynitrite (PN). In some embodiments, an increase in a level of endogenous GPX1 or GPX1-encoding nucleic acid (e.g., DNA or RNA) results in improved storage, aging, or circulation time of the enucleated erythroid cell. In some embodiments, one or both of redox capacity or scavenging of peroxides and PN in the erythroid cell, e.g., the enucleated erythroid cell, is increased relative to an unmodified otherwise similar erythroid cell. In some embodiments, an increase in the level of endogenous GPX1 or GPX1-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased redox capacity or scavenging of peroxides and PN. Assays for determining the redox state of cells are described in Nagababu et al. supra and Uchiyama et al. supra.

### Glutathione Peroxidase 4 (GPX4) Protein and Nucleic Acid

In some embodiments, an erythroid cell described herein has increased GPX4 compared to an otherwise similar unmodified cell. In some embodiments, the endogenous protein comprises endogenous glutathione peroxidase 4 (GPX4). In some embodiments, the endogenous nucleic acid (e.g., DNA or RNA) is a GPX4-encoding DNA or RNA. In some embodiments, the endogenous GPX4 protein is encoded by a nucleic acid comprising the sequence of Genbank Accession No. NM_002085.4, NM_001039847.2, or NM_001039848.3, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous GPX4-encoding nucleic acid is a naturally occurring allele or polymorph of the sequence of Genbank Accession No. NM_002085.4, NM_001039847.2, or NM_001039848.3, or a GPX4 protein encoded by the same.

In some embodiments, an increase in a level of endogenous GPX4 or GPX4-encoding nucleic acid (e.g., DNA or RNA) in an erythroid cell, e.g., an enucleated erythroid cell, results in one or both increased redox capacity or increased scavenging of peroxides and PN. In some embodiments, an increase in a level of endogenous GPX4 or GPX4-encoding nucleic acid (e.g., DNA or RNA) results in improved storage, aging, or circulation time of the erythroid cell. In some embodiments, one or both of redox capacity or scavenging of peroxides and PN in the erythroid cell, e.g., the enucleated erythroid cell, is increased relative to an unmodified otherwise similar erythroid cell. In some embodiments, an increase in the level of endogenous GPX4 or GPX4-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased redox capacity or scavenging of peroxides and PN. Assays for determining the redox state of cells are described, for example, in Rifkind & Nagababu (2013) 18(17), 2274-2283.

### Bim Protein and Nucleic Acid

In some embodiments, an erythroid cell described herein has decreased Bim compared to an otherwise similar unmodified cell. In some embodiments, the endogenous protein comprises endogenous Bim. In some embodiments, the endogenous nucleic acid (e.g., DNA or RNA) is a Bim-encoding DNA or RNA. In some embodiments, the endogenous Bim protein is encoded by a nucleic acid comprising the sequence of Genbank Accession No. AB071196.1, or an endogenous sequence with at least 70%, 75%, 80%, 85%, 90%, 95%, 96%, 97%, 98%, or 99% identity thereto, or an endogenous sequence with no more than 10, 9, 8, 7, 6, 5, 4, 3, 2, or 1 nucleic acid alterations relative thereto, e.g., substitutions, insertions, or deletions. In some embodiments, the endogenous Bim -encoding nucleic acid is a naturally occurring allele or polymorph of the sequence of Genbank Accession No. AB071196.1, or a Bim protein encoded by the same.

In some embodiments, a decrease in a level of endogenous Bim or Bim-encoding nucleic acid (e.g., DNA or RNA) in an erythroid cell, e.g., an enucleated erythroid cell, results in increased cell survival, e.g., during erythropoiesis and manufacturing (e.g., cell culture, expansion and differentiation in vitro). In some embodiments, the survival of the erythroid cell, e.g., the enucleated erythroid cell, is increased relative to an unmodified otherwise similar erythroid cell. In some embodiments, a decrease in the level of endogenous Bim or Bim-encoding RNA in the erythroid cell, e.g., the enucleated erythroid cell, is about 10%, 20%, 30%, 40%, 50%, 60%, 80%, 2-fold, 3-fold, 4-fold, 5-fold, or 10-fold compared to an unmodified otherwise similar erythroid cell.

In some embodiments, the erythroid cell, e.g., an enucleated erythroid cell, has activity of one or both of increased survival during erythropoiesis or increased survival during manufacturing (e.g., cell culture, expansion and differentiation in vitro). Survival of the enucleated erythroid cell comprising Bim can be measured, e.g., using an assay as described in Koulinis, M. (2012) Blood 119(5):1228-39, which is herein incorporated by reference in its entirety. Fold expansion and cell yield can be measured following ex vivo differentiation of CD34⁺ progenitor cells to enucleated erythroid cells.

### Proteins and Nucleic Acids (e.g., DNA or RNA) for Modulating Differentiation

In some embodiments, the endogenous protein is a protein, e.g., a transcription factor, that regulates self-renewal or differentiation of erythroid cells. In some embodiments, in order to promote self-renewal during an expansion phase of culturing, an erythroid precursor cell is treated with an agent (e.g., an exogenous nucleic acid-binding protein) that upregulates an endogenous gene that promotes self-renewal of erythroid precursor cells, or an agent that downregulates an endogenous gene that promotes differentiation. In some embodiments, in order to promote differentiation or maturation during a later stage in culture, the erythroid cell is treated with an agent (e.g., an exogenous nucleic acid-binding protein) that downregulates an endogenous gene that promotes self-renewal of erythroid precursor cells, or an agent that upregulates an endogenous gene that promotes differentiation. In some embodiments, the endogenous gene is selected from TERT (e.g., upregulated to promote self-renewal), Axl (e.g., upregulated to promote differentiation from erythroid progenitors, e.g., to pro-erythroblasts), EKLF/KLF1 (e.g., to modulate maturation, see Yang et al. (2017) Stem Cells 35(4): 886-97), BCL-XL (e.g., upregulated to inhibit apoptosis), c-MYC (e.g., upregulated to promote expansion and/or immortalization), SOX2 (e.g., upregulated to promote expansion and/or immortalization), TP53 (e.g., upregulated to promote expansion and/or immortalization), BMI1 (e.g., upregulated to promote expansion and/or immortalization), SH2B3 (e.g., upregulated to promote expansion, see Felix C. Giani, et al. (2016) Cell Stem Cell 18(1): 73-78), GATA1 (e.g., upregulated to promote erythroid differentiation and/or lineage commitment), GATA2 (e.g., upregulated to promote erythroid differentiation and/or lineage commitment), TAL1 (e.g., upregulated to promote erythroid differentiation and/or lineage commitment), or LMO2 (e.g., upregulated to promote erythroid differentiation and/or lineage commitment).

### Methods of Mediating Sequence-Specific Alterations in Gene Expression of Erythroid Cells

### CRISPR to Mediate a Sequence-Specific Alteration in Gene Expression of an Erythroid Cell

Artificial CRISPR polypeptide systems can be generated that mediate a sequence-specific alteration in gene expression of an endogenous protein in an erythroid cell, e.g., using technology described in U.S. Patent Publication No. 2014/0068797, and Cong et al. (2013) Science 339: 819-823, each of which is hereby incorporated by reference in its entirety. Other artificial CRISPR polypeptide systems may also be used to alter gene expression of an endogenous protein in an erythroid cell, e.g., as described in Tsai (2014) Nature Biotechnol., 32:6 569-576, and U.S. Pat. Nos. 8,871,445; 8,865,406; 8,795,965; 8,771,945; and 8,697,359, each of which is hereby incorporated by reference in its entirety.

A CRISPR polypeptide for use in an erythroid cell as described herein can be a naturally-occurring CRISPR polypeptide, e.g., from a eubacteria genome or an archaeal genome, as described in Grissa et al. (2007) BMC Bioinformatics 8: 172, which is hereby incorporated by reference in its entirety. CRISPR polypeptides are also described in Wiedenheft et al. (2012) Nature 482: 331-8, which is hereby incorporated by reference in its entirety.

RNA from the CRISPR locus is constitutively expressed and processed by Cas proteins into small RNAs comprising a spacer flanked by a repeat sequence. The small RNAs guide other Cas proteins, e.g., to silence exogenous genetic elements at the RNA or DNA level, as described in Horvath et al. (2010) Science 327: 167-170; Makarova et al. (2006) Biology Direct 1: 7, which is hereby incorporated by reference in its entirety. Thus, the spacers can serve as templates for RNA molecules in the erythroid cell, as described in Pennisi (2013) Science 341: 833-836, which is hereby incorporated by reference in its entirety.

The exact arrangements of the CRISPR and structure, function, and number of Cas genes and products thereof can differ from species to species (e.g., bacterial species), as described in Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340, each of which is hereby incorporated by reference in its entirety. For example, the Cse (Cas subtype, E. coli) proteins (e.g., CasA) can form a functional complex, Cascade, that processes CRISPR RNA transcripts into spacer-repeat units retaining Cascade, as described in Brouns et al. (2008) Science 321: 960-964, which is hereby incorporated by reference in its entirety. In other prokaryotes, Cas6 processes the CRISPR transcript. In some embodiments, the CRISPR-based phage inactivation in E. coli requires Cascade and Cas3, but does not Cas1 or Cas2. For example, the Cmr (Cas RAMP module) proteins in, e.g., Pyrococcus furiosus, form a functional complex with small CRISPR RNAs to recognize and cleave complementary target RNAs.

For example, a simpler CRISPR system relies on the protein Cas9. In some embodiments, a Cas9 protein is a nuclease with two active cutting sites to target each strand of double stranded DNA. Combining Cas9 and modified CRISPR locus RNA can be used for gene editing of, e.g., an erythroid cell, as described in Pennisi (2013) Science 341: 833-836, which is hereby incorporated by reference in its entirety.

In some embodiments, a S. pyogenes Cas9 polypeptide comprises motif 1 (Ile Gly Leu Asp Ile Gly Thr Asn Ser Val Gly Trp Ala Val Ile; SEQ ID NO: 1), motif 2 (Be Val Ile Glu Met Ala Arg Glu; SEQ ID NO: 2), motif 3 (Asp Val Asp His Be Val Pro Gln Ser Phe Leu Lys Asp Asp Ser Ile Asp Asn Lys Val Leu Thr Arg Ser Asp Lys Asn; SEQ ID NO: 3), and motif 4 (His His Ala His Asp Ala Tyr Leu; SEQ ID NO: 4). In some embodiments, motifs 1, 2, and 4 are RuvC like motifs and motif 3 is an HNH motif.

In some embodiments, the Cas9 protein is selected from: S. pyogenes Cas9 (wild type) (e.g., SEQ ID NO:848 of US Pat. App. Pub. 2017/0137801), S. pyogenes Cas9-mutation M1C (e.g., SEQ ID NO:849 of US Pat. App. Pub. 2017/0137801), S. pyogenes Cas9-mutation M1C & C80S (e.g., SEQ ID NOs:850 of US Pat. App. Pub. 2017/0137801), S. pyogenes Cas9 nickase-mutation D10A (e.g., SEQ ID NO:851 of US Pat. App. Pub. 2017/0137801), S. pyogenes Cas9 nickase-mutation H840A (e.g., SEQ ID NO:852 of US Pat. App. Pub. 2017/0137801), S. pyogenes Cas9 nickase-mutations E923P & T924P (SEQ ID NO:853 of US Pat. App. Pub. 2017/0137801), Acidovorax ebreus Cas9 (e.g., SEQ ID NO:854 of US Pat. App. Pub. 2017/0137801), Acid mine drainage bacteria Ga0052161_JGI Cas9 (e.g., SEQ ID NO:855 of US Pat. App. Pub. 2017/0137801), S. pyogenes Cas9 null-mutation D10A & H840A (SEQ ID NO:1027 of US Pat. App. Pub. 2017/0137801), and Uranium mine bacteria FW106_JGI Cas9 (SEQ ID NO:856 of US Pat. App. Pub. 2017/0137801) or a protein having at least 80%, 85%, 90%, 95%, 96%, 97%, 98%, or 99% identity thereto; each of said sequences is herein incorporated by reference in its entirety.

In some embodiments, the CRISPR protein is a Cpf1 protein. A Cpf1 protein may be derived from, e.g., Acidaminococcus sp., Lachnospiraceae bacterium, Porphyromonas macacae, or Prevotella disiens. In some embodiments, the Cpf1 protein is selected from, but is not limited to, Acidaminococcus sp Cpf1 (e.g., SEQ ID NO:857 of US Pat. App. Pub. 2017/0137801), L. bacterium Cpf1 (e.g., SEQ ID NO:858 of US Pat. App. Pub. 2017/0137801), P. macacae Cpf1 (e.g., SEQ ID NO:859 of US Pat. App. Pub. 2017/0137801), or P. disiens Cpf1 (e.g., SEQ ID NO:860 of US Pat. App. Pub. 2017/0137801) or a protein having at least 80%, 85%, 90%, 95%, 96%, 97%, 98%, or 99% identity thereto; each of said sequences is herein incorporated by reference in its entirety.

In some embodiments, the CRISPR polypeptide is less than 1,300, 1,200, 1,100, or 1,000 amino acids in length. Short Cas9 proteins include a Staphylococcus aureus Cas9 (SaCas9) and other Cas9 proteins, e.g., as described in Ann Ran et al. (2015) Nature 520: 186-191 and US Pat. Appl. Pub. No. 2017/0349914, each of which is herein incorporated by reference in its entirety.

Split CRISPR peptides can also be used. These can be used, e.g., to circumvent size restrictions in delivery vectors, or to induce activity at a desired time. As an example, a split Cas9 inducible with rapamycin can feature a first fusion protein comprising a first fragment of Cas9 with FRB, and a second fusion protein comprising a second fragment of Cas9 with FKBP. Addition of rapamycin promotes Cas9 activity, e.g., by promoting dimerization of the FRB and FKBP domains. As another example, a split Cas9 inducible with light can feature a first fusion protein comprising a first fragment of Cas9 with CIB1, and a second fusion protein comprising a second fragment of Cas9 with CRY2. Exposure of the protein to blue light promotes Cas9 activity, e.g., by promoting dimerization of the CIB1 and CRY2 domains. In some embodiments, a split Cas9 is introduced into a nucleated erythroid precursor cell, and then an inducer (e.g., rapamycin or light) is provided at a desired time, e.g., during expansion, differentiation, or maturation.

A CRISPR polypeptide can thus be used to edit an endogenous gene or promoter present in an erythroid cell (e.g., by adding or deleting a base pair), or introducing a premature stop codon, thereby decreasing expression of an endogenous gene. In other embodiments, a CRISPR polypeptide can edit the genome to upregulate expression of an endogenous gene, e.g., by inserting a strong promoter such that it is operably linked to (e.g., precedes the start) of the gene, e.g., using homologous recombination based on a template carrying the promoter of interest.

The CRISPR/Cas system can alternatively be used to upregulate or downregulate expression of an endogenous gene of the enucleated erythroid cell in a reversible fashion. For example, the RNA can guide the Cas protein to a promoter of an endogenous gene of interest in an erythroid cell, thereby sterically blocking RNA polymerases. As an alternative, the CRISPR protein, e.g., a catalytically inactive CRISPR protein, can be fused to a transcriptional activator or repressor domain, e.g., in order to upregulate or downregulate transcription at the locus it binds.

In some embodiments, the CRISPR polypeptide is co-expressed with (e.g., forms a complex with) a guide RNA, e.g., a guide RNA or a single guide RNA, e.g., as described herein. Without wishing to be bound by theory, in some embodiments, a guide RNA confers specificity to a protein such as Cas9, by the gRNA binding to a DNA sequence of interest and also binding to the Cas9 protein. Thus, in some embodiments, methods herein comprise introducing a gRNA or DNA encoding the gRNA into a cell, and also introducing into the cell a nucleic acid encoding a CRISPR polypeptide. Methods for designing guide RNAs (e.g., gRNAs or sgRNAs) are known in the art. Methods for selection and validation of a target sequence present on an endogenous gene, as well as off-target analyses, are described, e.g., in Mali et al. (2013) Science 339(6121): 823-826; Hsu et al. (2013) Nat. Biotechol. 31(9): 827-32; Fu et al. (2014) Nat. Biotechol. 32(3): 279-284; Heigwer et al. (2014) Nat. Methods 11(2):122-3; and Bae et al. (2014) Bioinformatics 30(10): 1473-5. For example, software tools can be used to optimize the choice of a guide RNA (e.g., gRNA) within a user's target sequence on an endogenous gene, e.g., to minimize total off-target activity across the genome.

### TALE Proteins and TALEN to Mediate a Sequence-Specific Alteration in Gene Expression of an Erythroid Cell

In some embodiments, TALENs for use in an erythroid cell as described herein can be produced artificially by fusing a TAL effector DNA binding domain to a DNA cleavage domain. The transcription activator-like effects (TALEs) can be engineered to bind any desired DNA sequence such as a portion of the gene encoding the endogenous protein of the erythroid cell. By combining an engineered TALE with a DNA cleavage domain, a restriction enzyme can be produced which is specific to any desired DNA sequence (e.g., an endogenous DNA) of the erythroid cell. These can then be introduced into a cell for genome editing, e.g., using methods described in Boch (2011) Nature Biotech. 29: 135-6; and Boch et al. (2009) Science 326: 1509-12; Moscou et al. (2009) Science 326: 3501, each of which is hereby incorporated by reference in its entirety.

TALEs are proteins secreted by Xanthomonas bacteria. The DNA binding domain contains a repeated, highly conserved 33-34 amino acid sequence except for the twelfth and thirteenth amino acids, which are highly variable and exhibit specific nucleotide recognition. Thus, TALEs can be engineered to bind to a desired endogenous DNA sequence in an erythroid cell.

To produce a TALEN, a TALE protein can be fused to a nuclease (N), e.g., a wild-type or mutated FokI endonuclease. Mutations to FokI for use in TALENs, e.g., mutations to improve cleavage specificity or activity, are described in Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96, each of which is hereby incorporated by reference in its entirety.

The FokI domain functions as a dimer and often involves two constructs with unique DNA binding domains to target sites in the genome of the erythroid cell with proper orientation and spacing. For example, the number of amino acid residues between the TALE DNA binding domain and the FokI cleavage domain and the number of bases between the two individual TALEN binding sites may be important to achieve high levels of activity, e.g., in the erythroid cell, as described in Miller et al. (2011) Nature Biotech. 29: 143-8.

A TALEN targeting an endogenous gene of an erythroid cell can be used in the erythroid cell to produce a double-stranded break (DSB). For example a mutation can be introduced at the double-stranded break site by the repair mechanisms improperly repairing the break via non-homologous end joining, e.g., improper repair may introduce a frame shift mutation or a deletion within a promoter region. Alternatively, foreign DNA can be introduced into the erythroid cell along with the TALEN, which can be used to alter the sequence of an endogenous nucleic acid, e.g., a promoter or coding region, thereby altering expression of the endogenous protein of the erythroid cell.

TALE proteins can alternatively be used to upregulate or downregulate expression of an endogenous gene of the enucleated erythroid cell in a reversible fashion. For example, the TALE protein can bind to a promoter of an endogenous gene of interest in an erythroid cell, thereby sterically blocking RNA polymerases. As an alternative, the TALE protein can be fused to a transcriptional activator or repressor domain, e.g., in order to upregulate or downregulate transcription at the locus it binds.

TALENs specific to nucleic acid sequences of the endogenous proteins described herein can be constructed using any suitable method, including those described in Zhang et al. (2011) Nature Biotech. 29: 149-53; Geibler et al. (2011) PLoS ONE 6: e19509, each of which is hereby incorporated by reference in its entirety.

### ZF Proteins and ZFN to Mediate a Sequence-Specific Alteration in Gene Expression of an Erythroid Cell

A ZFN for use in an erythroid cell can comprise a FokI nuclease domain (or derivative thereof) fused to a DNA-binding domain, which comprises one or more zinc fingers, as described in Carroll et al. (2011) Genetics Society of America 188: 773-782; and Kim et al. (1996) Proc. Natl. Acad. Sci. USA 93: 1156-1160, each of which is hereby incorporated by reference in its entirety.

A ZF is a small protein structural motif stabilized by one or more zinc ions, which can comprise, e.g., Cys₂His₂, and can recognize an approximately 3-bp nucleic acid sequence. Various zinc fingers of known specificity can be combined to produce multi-finger polypeptides that recognize nucleic acid sequences of about 6, 9, 12, 15 or 18-bp. For example, selection and modular assembly techniques available to generate ZFs recognizing specific sequences of an erythroid cell include the following: phage display, yeast one-hybrid systems, bacterial one-hybrid and two-hybrid systems, mammalian cells, and combinations thereof.

A pair of ZFNs can be used to target non-palindromic DNA sites because a ZFN typically dimerizes to cleave the gene of an erythroid cell. The two individual ZFNs bind opposite strands of the DNA with proper spacing of the nucleases, as described in Bitinaite et al. (1998) Proc. Natl. Acad. Sci. USA 95: 10570-5, which is hereby incorporated by reference in its entirety.

A ZFN targeting an endogenous gene of an erythroid cell can be used to produce a DSB in the DNA of the erythroid cell. For example, a double-stranded break in the DNA can create a frame-shift mutation due to improper repair of the DNA, thereby decreasing expression of the endogenous protein in the erythroid cell. ZFNs can also be used with homologous recombination to mutate the endogenous gene of the erythroid cell.

ZF proteins can alternatively be used to upregulate or downregulate expression of an endogenous gene of the enucleated erythroid cell in a reversible fashion. For example, the ZF protein can bind to a promoter of an endogenous gene of interest in an erythroid cell, thereby sterically blocking RNA polymerases. As an alternative, the ZF protein can be fused to a transcriptional activator or repressor domain, e.g., in order to upregulate or downregulate transcription at the locus it binds.

ZFNs specific to sequences of endogenous proteins of the erythroid cell described herein can be constructed, e.g., as described in Provasi (2011) Nature Med. 18: 807-815; Torikai (2013) Blood 122: 1341-1349; Cathomen et al. (2008) Mol. Ther. 16: 1200-7; Guo et al. (2010) J. Mol. Biol. 400: 96; U.S. Patent Publication 2011/0158957; and U.S. Patent Publication 2012/0060230, each of which is hereby incorporated by reference in its entirety.

### Adeno-Associated Virus (AAV) Vectors to Mediate a Sequence-Specific Alteration in Gene Expression of an Erythroid Cell

Adeno-associated virus uses homologous recombination, directed by a pair of homology arms, to replace an endogenous nucleic acid sequence with the nucleic acid sequence between the homology arms of the AAV vector. Thus, an AAV vector can be used to insert, delete, or replace an endogenous nucleic acid sequence.

In some embodiments, the AAV vector is single stranded linear DNA. In some embodiments, the AAV vector comprises a selectable marker, e.g., to allow selection of cells that successfully underwent homologous recombination. In some embodiments, the selectable marker is flanked by loxP sites, e.g., to allow removal of the marker after selection is complete. In some embodiments, the AAV comprises an inverted terminal repeat at each end of the vector, e.g., to allow replication. In some embodiments, the AAV lacks rep and/or cap genes.

AAV can also be used in combination with a site-specific nuclease (e.g., a TALEN polypeptide, a ZFN polypeptide, or a CRISPR polypeptide). In some embodiments, the site-specific nuclease creates a double stranded break in the target sequence, and the AAV serves as a template for homology-directed repair of the break.

In some embodiments, AAV is used to modify gene expression in an erythroid precursor cell as described herein, e.g., by removing or replacing a promoter. In some embodiments, AAV is used as described herein to alter a coding sequence.

AAV, as a stand-alone technology and in combination with site-specific nucleases, is described, e.g., in Gaj et al. (2016) Mol Ther. 24(3): 458-464, which is herein incorporated by reference in its entirety.

### Tuning Transcriptional Activation or Repression

The timing of gene regulation can be controlled as described herein. For instance, a nucleic acid (e.g., mRNA) encoding a site-specific DNA binding protein can be introduced into an erythroid cell at a desired time, e.g., during expansion, differentiation, or maturation. The level and timing of administration can be chosen such that expression is temporally limited. As an example, an mRNA encoding a factor that promotes self-renewal can be introduced into an erythroid cell during expansion phase. Alternatively or in combination, an mRNA encoding a factor that promotes differentiation can be introduced into an erythroid cell during or just before differentiation phase.

As another approach to temporal regulation, an inducible site-specific DNA binding protein can be used. Exemplary split Cas9 proteins are described in the section herein entitled “CRISPR to mediate a sequence-specific alteration in gene expression of an erythroid cell”. It is understood that embodiments which call for a site-specific DNA binding protein can also use a pair of proteins which complex when an inducer is present. In some embodiments, the inducer is added during expansion, differentiation, or maturation phase. Concentration of the inducing molecule can be modulated to achieve a desired gene expression level.

### Physical Characteristics of Enucleated Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an enucleated erythroid cell that expresses an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the enucleated erythroid cell comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the enucleated erythroid cell exhibits substantially the same osmotic membrane fragility as an isolated, uncultured erythroid cell that does not comprise an exogenous polypeptide. In some embodiments, the population of enucleated erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the enucleated erythroid cell has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the enucleated erythroid cell has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the enucleated erythroid cell has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of International Patent Publication No. WO 2015/073587, which is herein incorporated by reference in its entirety.

Other Characteristics

In some embodiments, an erythroid cell (e.g., enucleated erythroid cell), or a population of erythroid cells comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA⁺ (i.e., CD235a⁺) cells. In some embodiments, the population of enucleated erythroid cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA⁺ cells. The presence of GPA is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD47⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD36⁻ (CD36-negative) cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD36⁻ (CD36-negative) cells. The presence of CD36 is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD34⁻ (CD34-negative) cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD34⁻ (CD34-negative) cells. The presence of CD34 is detected, in some embodiments, using FACS.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%, CD235a⁺/CD47⁺/CD233⁺ cells.

In some embodiments, the population of enucleated erythroid cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺/CD34⁻/CD36⁻ cells. In some embodiments, the population of enucleated erythroid cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/CD34⁻/CD36⁻ cells.

In some embodiments, a population of enucleated erythroid cells comprising erythroid cells comprises less than about 10%, 9%, 8%, 7%, 6%, 5%, 4%, 3%, 2%, or 1% echinocytes.

In some embodiments, a population of enucleated erythroid cells comprises less than about 10%, 9%, 8%, 7%, 6%, 5%, 4%, 3%, 2%, or 1% pyrenocytes.

In some embodiments, the erythroid cells have a half-life of at least 0.5, 1, 2, 7, 14, 30, 45, or 90 days in a subject.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell, e.g., an erythroid cell, contains a nucleus that is non-functional, e.g., has been inactivated.

### Universal Donor Erythroid Cells

In some embodiments, erythroid cells described herein are autologous or allogeneic to the subject to which the cells will be administered. For example, erythroid cells allogeneic to the subject include one or more of blood type specific erythroid cells (e.g., the cells can be of the same blood type as the subject) or one or more universal donor erythroid cells. In some embodiments, the enucleated erythroid cells described herein have reduced immunogenicity compared to a reference cell, e.g., have lowered levels of one or more blood group antigens.

Where allogeneic cells are used for transfusion, a compatible ABO blood group can be chosen to prevent an acute intravascular hemolytic transfusion reaction. The ABO blood types are defined based on the presence or absence of the blood type antigens A and B, monosaccharide carbohydrate structures that are found at the termini of oligosaccharide chains associated with glycoproteins and glycolipids on the surface of the erythrocytes (reviewed in Liu et al., Nat. Biotech. 25:454-464 (2007)). Because group O erythrocytes contain neither A nor B antigens, they can be safely transfused into recipients of any ABO blood group, e.g., group A, B, AB, or O recipients. Group O erythrocytes are considered universal and may be used in all blood transfusions. Thus, in some embodiments, an erythroid cell described herein is type O. In contrast, group A erythroid cells may be given to group A and AB recipients, group B erythroid cells may be given to group B and AB recipients, and group AB erythroid cells may be given to AB recipients.

In some instances, it may be beneficial to convert a non-group O erythroid cell to a universal blood type. Enzymatic removal of the immunodominant monosaccharides on the surface of group A and group B erythrocytes may be used to generate a population of group O-like erythroid cells (See, e.g., Liu et al., Nat. Biotech. 25:454-464 (2007)). Group B erythroid cells may be converted using an α-galactosidase derived from green coffee beans. Alternatively or in addition, α-N-acetylgalactosaminidase and α-galactosidase enzymatic activities derived from E. meningosepticum bacteria may be used to respectively remove the immunodominant A and B antigens (Liu et al., Nat. Biotech. 25:454-464 (2007)), if present on the erythroid cells. In one example, packed erythroid cells isolated as described herein, are incubated in 200 mM glycine (pH 6.8) and 3 mM NaCl in the presence of either α-N-acetylgalactosaminidase and α-galactosidase (about 300 μg/ml packed erythroid cells) for 60 min at 26° C. After treatment, the erythroid cells are washed by 3-4 rinses in saline with centrifugation and ABO-typed according to standard blood banking techniques.

While the ABO blood group system is the most important in transfusion and transplantation, in some embodiments it can be useful to match other blood groups between the erythroid cells to be administered and the recipient, or to select or make erythroid cells that are universal for one or more other (e.g., minor) blood groups. A second blood group is the Rh system, wherein an individual can be Rh+ or Rh−. Thus, in some embodiments, an erythroid cell described herein is Rh−. In some embodiments, the erythroid cell is Type O and Rh−.

In some embodiments, an erythroid cell described herein is negative for one or more minor blood group antigens, e.g., Le(a−b−) (for Lewis antigen system), Fy(a−b−) (for Duffy system), Jk(a−b−) (for Kidd system), M−N− (for MNS system), K−k− (for Kell system), Lu(a−b−) (for Lutheran system), and H-antigen negative (Bombay phenotype), or any combination thereof. In some embodiments, the erythroid cell is also Type O and/or Rh−. Minor blood groups are described, e.g., in Agarwal et al “Blood group phenotype frequencies in blood donors from a tertiary care hospital in north India” Blood Res. 2013 March; 48(1): 51-54 and Mitra et al “Blood groups systems” Indian J Anaesth. 2014 September-October; 58(5): 524-528, each of which is incorporated herein by reference in its entirety.

### Methods of Manufacturing Enucleated Erythroid Cells

Methods of manufacturing enucleated erythroid cells comprising (e.g., expressing) an exogenous agent (e.g., polypeptides) are described, e.g., in International Patent Publication Nos. WO2015/073587 and WO2015/153102, each of which is incorporated by reference in its entirety.

In some embodiments, hematopoietic progenitor cells, e.g., CD34+ hematopoietic progenitor cells, are contacted with a nucleic acid or nucleic acids encoding one or more exogenous polypeptides, and the cells are allowed to expand and differentiate in culture. In some embodiments, the CD34+ cells are immortalized, e.g., comprise a human papilloma virus (HPV; e.g., HPV type 16) E6 and/or E7 genes. In some embodiments, the immortalized CD34+ hematopoietic progenitor cell is a BEL-A cell line cell (see Trakarnasanga et al. (2017) Nat Commun. 8: 14750). Additional immortalized CD34+ hematopoietic progenitor cells are described in U.S. Pat. Nos. 9,951,350, and 8,975,072. In some embodiments, an immortalized CD34+ hematopoietic progenitor cell is contacted with a nucleic acid or nucleic acids encoding one or more exogenous polypeptides, and the cells are allowed to expand and differentiate in culture.

In some embodiments, the erythroid cells described herein are made by a method comprising contacting a nucleated erythroid cell, or precursor thereof, with an exogenous nucleic acid. The exogenous nucleic acid may be a nucleic acid that is not produced by a wild-type cell of that type or is present at a lower level in a wild-type cell than in a cell containing the exogenous nucleic acid. In some embodiments, the exogenous nucleic acid is codon-optimized. For instance, the exogenous nucleic acid may comprise one or more codons that differ from the wild-type codons in a way that does not change the amino acid encoded by that codon, but that increases translation of the nucleic acid, e.g., by using a codon preferred by the host cell, e.g., a mammalian cell, e.g., an erythroid cell.

The method may further comprise culturing the nucleated erythroid cell, or precursor thereof, under conditions suitable for expression of the exogenous protein and/or for enucleation.

In some embodiments, the two or more polypeptides are encoded in a single nucleic acid, e.g. a single vector. In some embodiments, the single vector has a separate promoter for each gene, has two proteins that are initially transcribed into a single polypeptide having a protease cleavage site in the middle, so that subsequent proteolytic processing yields two proteins, or any other suitable configuration. In some embodiments, the two or more polypeptides are encoded in two or more nucleic acids, e.g., each vector encodes one of the polypeptides.

The nucleic acid may be, e.g., DNA or RNA. A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, the exogenous nucleic acid is operatively linked to a constitutive promoter. In some embodiments, a constitutive promoter is used to drive expression of the targeting moiety.

In some embodiments, the exogenous nucleic acid is operatively linked to an inducible or repressible promoter, e.g., to drive expression of the amino acid degradative enzyme. For instance, the promoter may be doxycycline-inducible, e.g., a P-TRE3GS promoter or active fragment or variant thereof. Examples of inducible promoters include, but are not limited to a metallothionine-inducible promoter, a glucocorticoid-inducible promoter, a progesterone-inducible promoter, and a tetracycline-inducible promoter (which may also be doxycycline-inducible). In some embodiments, the inducer is added to culture media comprising cells that comprise the inducible promoter, e.g., at a specific stage of cell differentiation. In some embodiments, the inducer (e.g., doxycycline) is added at an amount of about 1-5, 2-4, or 3 μg/mL. In some embodiments, a repressor is withdrawn from to culture media comprising cells that comprise the repressible promoter, e.g., at a specific stage of cell differentiation. In some embodiments, the inducer is added, or the repressor is withdrawn, during maturation phase, e.g., between days 1-10, 2-9, 3-8, 4-6, or about day 5 of maturation phase. In some embodiments, the inducer is present, or the repressor is absent, between day 1, 2, 3, 4, 5, 6, 7,8, 9 or 10 of maturation and enucleation. In some embodiments, the inducer is present, or the repressor is absent, for at least 1, 2, 3, 4, 5, 6, 7, 8, 9, or 10 days. In some embodiments, the inducer is present, or the repressor is absent, from maturation day 5 to the end of differentiation. In some embodiments, the inducer is present, or the repressor is absent at maturation day 9. In some embodiments, the inducer is added, or the repressor is withdrawn, when the population of erythroid cells comprises a plurality of normoblasts (e.g., basophilic, polychromatic, or orthochromatic normoblasts or a combination thereof), e.g., when 10-20%, 20-30%, 30-40%, 40-50%, 50-60%, 60-70%, or 70-80% of the cells in the population are normoblasts. In some embodiments, the inducer is added, or the repressor is withdrawn, when the population of erythroid cells comprises a plurality of pro-erythroblasts, e.g., when 10-20%, 20-30%, 30-40%, 40-50%, 50-60%, 60-70%, or 70-80% of the cells in the population are pro-erythroblasts. In some embodiments, the inducer is added, or the repressor is withdrawn, when the population of erythroid cells comprises a plurality of erythroblasts at terminal differentiation e.g., when 10-20%, 20-30%, 30-40%, 40-50%, 50-60%, 60-70%, or 70-80% of the cells in the population are erythroblasts at terminal differentiation. In some embodiments, the erythroid cell or population of erythroid cells comprises an additional exogenous protein, e.g., a transactivator, e.g., a Tet-inducible transactivator (e.g., a Tet-on-3G transactivator).

In some embodiments, the inducer is added, or the repressor is withdrawn, when the population of erythroid cells comprises one or more of (e.g., all of) endogenous GPA, band3, or alpha4 integrin. In some embodiments, the inducer is added, or the repressor is withdrawn, during a time when about 84-100%, 85-100%, 90-100%, or 95-100% of the cells in the population are GPA-positive (e.g., when the population first reaches that level); during a time when 50-100%, 60-100%, 70-100%, 80-100%, 90-100%, 95-100%, or 98-100% of the cells in the population are band3-positive (e.g., when the population first reaches that level); and/or during a time when about 70-100%, 80-90%, or about 85% of the cells in the population are alpha4 integrin-positive (e.g., when the population first reaches that level).

GPA, band3, and alpha4 integrin can be detected, e.g., by a flow cytometry assay, e.g., a flow cytometry assay of Example 10 of International Application Publication No. WO2018/009838, which is incorporated herein by reference in its entirety.

In some embodiments, the cells are produced using conjugation, e.g., sortagging, e.g., as described in WO2014/183071 or WO2014/183066, each of which is incorporated by reference in its entirety. In some embodiments, the cells are made by a method that does not comprise sortase-mediated conjunction.

In some embodiments, the cells are made by a method that does not comprise hypotonic loading. In some embodiments, the cells are made by a method that does not comprise a hypotonic dialysis step.

In some embodiments, a site-specific DNA binding protein described herein is introduced into a cell as a purified protein, e.g., using electroporation or as part of an LNP.

In some embodiments, the erythroid cells are expanded at least 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, the population of erythroid cells comprises at least about 30%, 35%, 40%, 45%, 50%, 55%, 60%, 65%, 70%, 75%, 80%, 85%, 90%, 95%, 96% or 98% (and optionally up to about 80%, 90%, or 100%) enucleated erythroid cells. In some embodiments, the population of erythroid cells comprises 70%400%, 75%-100%, 80%-100%, 85%-100%, or 90%-100% enucleated cells. In some embodiments, the population of erythroid cells contains less than 1% live nucleated cells, e.g., contains no detectable live nucleated cells. Enucleation is measured, in some embodiments, by FACS using a nuclear stain. In some embodiments, at least about 30%, 35%, 40%, 45%, 50%, 55%, 60%, 65%, 70%, 75%, 80%, 85%, 90%, 95%, 96% or 98% (and optionally up to about 70%, 80%, 90%, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least about 30%, 35%, 40%, 45%, 50%, 55%, 60%, 65%, 70%, 75%, 80%, 85%, 90%, 95%, 96% or 98% (and optionally up to about 70%, 80%, 90%, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹- 1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, an enucleated cell provided herein is a platelet. Methods of manufacturing platelets in vitro are known in the art (see, e.g., Wang and Zheng (2016) Springerplus 5(1): 787, and U.S. Pat. No. 9,574,178). Methods of manufacturing platelets including an exogenous polypeptide are described, e.g., in International Patent Application Publication Nos. WO2015/073587 and WO2015/153102, each of which is incorporated by reference in its entirety. Platelet production is in part regulated by signaling mechanisms induced by interaction between thrombopoietin (TPO) and its cellular receptor TPOR/MPUc-MPL. In addition, multiple cytokines (e.g., stem cell factor (SCF), IL-1, IL-3, IL-6, IL-11, leukemia inhibiting factor (LIF), G-CSF, GM-CSF, M-CSF, erythropoietin (EPO), kit ligand, and interferon) have been shown to possess thrombocytopoietic activity.

In some embodiments, platelets are generated from hematopoietic progenitor cells, such as CD34⁺ hematopoietic stem cells, induced pluripotent stem cells or embryonic stem cells. In some embodiments, platelets are produced by contacting the progenitor cells with defined factors in a multi-step culture process. In some embodiments, the multi-step culture process comprises: culturing a population of hematopoietic progenitor cells under conditions suitable to produce a population of megakaryocyte progenitor cells, and culturing the population of megakaryocyte progenitor cells under conditions suitable to produce platelets. Cocktails of growth and differentiation factors that are suitable to expand and differentiate progenitor cells and produce platelets are known in the art. Examples of suitable expansion and differentiation factors include, but are not limited to, stem cell factor (SCF), Flt-3/Flk-2 ligand (FL), TPO, IL-11, IL-3, IL-6, and IL-9. For instance, in some embodiments, platelets may be produced by seeding CD34⁺ HSCs in a serum-free medium at 2-4×10⁴ cells/mL, and refreshing the medium on culture day 4 by adding an equal volume of media. On culture day 6, cells are counted and analyzed: 1.5×10⁵ cells are washed and placed in 1 mL of the same medium supplemented with a cytokine cocktail comprising TPO (30 ng/mL), SCF (1 ng/mL), IL-6 (7.5 ng/mL), and IL-9 (13.5 ng/mL) to induce megakaryocyte differentiation. At culture day 10, from about one quarter to about half of the suspension culture is replaced with fresh media. The cells are cultured in a humidified atmosphere (10% CO₂) at 39° C. for the first 6 culture days, and at 37° C. for the last 8 culture days. Viable nucleated cells are counted with a hemocytometer following trypan blue staining. The differentiation state of platelets in culture can be assessed by flow cytometry or quantitative PCR as described in Examples 44 and 45 of in International Patent Application Publication No. WO2015/073587, incorporated herein by reference.

### Vehicles for Polypeptides Described Herein

While in many embodiments herein, the one or more exogenous polypeptides are situated on or in an enucleated erythroid cell, it is understood that any polypeptide or combination of exogenous polypeptides described herein can also be situated on or in another vehicle. The vehicle can comprise, e.g., a cell, an erythroid cell, a platelet, a corpuscle, a nanoparticle, a micelle, a liposome, or an exosome. For instance, in some aspects, the present disclosure provides a vehicle (e.g., a cell, an erythroid cell, a platelet, a corpuscle, a nanoparticle, a micelle, a liposome, or an exosome) comprising, e.g., on its surface, one or more exogenous polypeptides described herein. In some embodiments, the one or more exogenous polypeptides comprise a polypeptide described in any of Tables 1-3 or a fragment or variant thereof, or an antibody molecule thereto.

In some embodiments, the vehicle comprises an erythroid cell. In some embodiments, the erythroid cell is a nucleated red blood cell, red blood cell precursor, or enucleated red blood cell. In some embodiments, the erythroid cell is a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, or a combination thereof. In some embodiments, the erythroid cells are immortal or immortalized cells.

### Cells Encapsulated in a Membrane

In some embodiments, enucleated erythroid cells or other vehicles described herein are encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. In some embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

Methods of Treatment with Compositions Herein, e.g., Enucleated Erythroid Cells

Methods of administering enucleated erythroid cells (e.g., reticulocytes) comprising (e.g., expressing) exogenous agent (e.g., polypeptides) are described, e.g., in WO2015/073587 and WO2015/153102, each of which is incorporated by reference in its entirety.

In some embodiments, the enucleated erythroid cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications.

In some embodiments, the erythroid cells are administered to a subject every 1, 2, 3, 4, 5, or 6 months.

In some embodiments, a dose of erythroid cells comprises about 1×10⁹-2×10⁹, 2×10⁹2×10⁹-5×10⁹, 5×10⁹-1×10¹⁰, 1×10¹⁰-2×10¹⁰, 2×10¹⁰5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, the erythroid cells are administered to a subject in a dosing regimen (dose and periodicity of administration) sufficient to maintain function of the administered erythroid cells in the bloodstream of the subject over a period of 2 weeks to a year, e.g., one month to one year or longer, e.g., at least 2 weeks, 4 weeks, 6 weeks, 8 weeks, 3 months, 6 months, a year, 2 years.

In some embodiments, enucleated erythroid cells described herein are administered to treat an autoimmune disease. In some embodiments, enucleated erythroid cells described herein are administered to treat a disease listed in Table 4. In some embodiments, an erythroid cell having reduced CD47 levels (e.g., substantially lacking CD47), or lacking CD47, and comprising an exogenous antigen of Table 4 is administered to treat a disease of Table 4. In some embodiments, an erythroid cell having reduced CD47 levels (e.g., substantially lacking CD47) and comprising an exogenous antigen of Table 4 is administered to induce immune tolerance to the antigen (e.g., in a subject having a disease of Table 4).

In some embodiments, the erythroid cell comprises 4-1BBL, anti-CD20, TRAIL, anti-PD-L1, or asparaginase, and can be used to treat cancer in a subject in need thereof. In some embodiments, the erythroid cells comprises phenylalanine ammonia lyase (PAL) and can be used to reduce phenylalanine levels, e.g., treat phenylketonuria (PKU) or hyperphenylalaninemia, in a subject in need thereof. In some embodiments, the erythroid cells comprises clotting factor X and are used to treat a clotting disease in a subject in need thereof.

### TABLES

TABLE 1
Exemplary endogenous genes to upregulate in erythroid cells

| | Exemplary | |
|---|---|---|
| | Genbank | |
| Gene name | Accession No. | Exemplary effects of upregulation |
| CD58 | Y14780.1 | Increase immunogenicity, e.g., by preventing |
| | | CD8+ T cell exhaustion. |
| CR1 | AL137789.11 | Bind and clear immune complexes from |
| | | circulation, e.g., to treat autoimmune disease |
| CD47 | AK289813.1 | Negatively regulate phagocytosis; prolong |
| | | circulating time; increase tolerogenic potential |
| ATP11C (Flippase) | AL161777.4 | Reduce PS exposure in membrane; increase |
| | | circulating time |
| CD55 | AB240567.1 | Inhibit complement activation; increase |
| | | circulating time |
| CD59 | X17198.1 | Inhibit complement activation; increase |
| | | circulating time |
| Bcl-xL | CR936637.1 | Increase cell survival, e.g., during erythropoiesis |
| | | and manufacturing |
| VKORC1 (Vitamin K | AY466113.1 | Increase gla domain posttranslational |
| Epoxide Reductase | | modification of coagulation factors |
| Complex) | | |
| GGCX (Gamma- | AK297397.1 | Increase gla domain posttranslational |
| carboxylase) | | modification of coagulation factors |
| CYB5R3 | AJ010116.1 | Increase redox capacity of cells, e.g., improve |
| | | storage, reduce cell aging, and increase |
| | | circulation time |
| SOD1 | X01780.1 | Increase redox capacity of cells, e.g., improve |
| | | storage, reduce cell aging, and increase |
| | | circulation time |
| GPX1 | X13710.1 | Increase redox capacity of cells, e.g., improve |
| | | storage, reduce cell aging, and increase |
| | | circulation time |
| GPX4 (glutathione | NM_002085.4 | Increase redox capacity of cells, e.g., improve |
| peroxidase) | | storage, reduce cell aging, and increase |
| | | circulation time |
| PRDX2 | CR541789.1 | Increase redox capacity of cells, e.g., improve |
| (peroxiredoxin) | | storage, reduce cell aging, and increase |
| | | circulation time |

TABLE 2
Exemplary endogenous genes to downregulate in erythroid cells

| | Exemplary | |
|---|---|---|
| | Genbank | |
| Gene name | Accession No. | Exemplary effects of downregulation |
| CD58 | Y14780.1 | Decrease immunogenicity |
| CD47 | AK289813.1 | Drive clearance; target to macrophages |
| PLSCR1 | AK300181.1 | Reduce PS exposure in membrane; |
| (Scramblase) | | increase circulating time |
| Bim | AB071196.1 | Reduce apoptosis; increase cell |
| | | survival, e.g., during erythropoiesis |
| | | and manufacturing. |

TABLE 3
Exemplary exogenous polypeptides for expression in erythroid cells

| Protein | |
|---|---|
| name | Sequence |
| 4-1BBL | ACPWAVSGARASPGSAASPRLREGPELSPDDPAGLLDLRQGMFAQLVAQN |
| | VLLIDGPLSWYSDPGLAGVSLTGGLSYKEDTKELVVAKAGVYYVFFQLEL |
| | RRVVAGEGSGSVSLALHLQPLRSAAGAAALALTVDLPPASSEARNSAFGFQ |
| | GRLLHLSAGQRLGVHLHTEARARHAWQLTQGATVLGLFRVTPEIPAGLPSP |
| | RSE (SEQ ID NO: 5) |
| Anti- | Rituximab heavy chain chimeric: |
| CD20 | QVQLQQPGAELVKPGASVKMSCKASGYTFTSYNMHWVKQTPGRGLEWIG |
| | AIYPGNGDTSYNQKFKGKATLTADKSSSTAYMQLSSLTSEDSAVYYCARST |
| | YYGGDWYFNVWGAGTTVTVSAASTKGPSVFPLAPSSKSTSGGTAALGCLV |
| | KDYFPEPVTVSWNSGALTSGVHTFPAVLQSSGLYSLSSVVTVPSSSLGTQTY |
| | ICNVNHKPSNTKVDKKAEPKSCDKTHTCPPCPAPELLGGPSVFLFPPKPKDT |
| | LMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK (SEQ |
| | ID NO: 6) |
| | Rituximab light chain chimeric: |
| | QIVLSQSPAILSASPGEKVTMTCRASSSVSYIHWFQQKPGSSPKPWIYATSNL |
| | ASGVPVRFSGSGSGTSYSLTISRVEAEDAATYYCQQWTSNPPTFGGGTKLEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKS |
| | FNRGEC (SEQ ID NO: 7) |
| TRAIL | Soluble TRAIL variant DR4-1 |
| | MAMMEVQGGPSLGQTCVLIVIFTVLLQSLCVAVTYVYFTNELKQMQDKYS |
| | KSGIACFLKEDDSYWDPNDEESMNSPCWQVKWQLRQLVRKMILRTSEETIS |
| | TVQEKQQNISPLVRERGPQRVAAHITGTRRRSNTLSSPNSKNEKALGRKINS |
| | WESSRSGHSFLSNLHLRNGELVIHEKGFYYIYSQTYFRFQEEIKENTKNDKQ |
| | MVQYIYKYTSYPDPILLMKSARNSCWSKDAEYGLYSIYQGGIFELKENDRIF |
| | VSVTNEHLIDMDHEASFFGAFLVG (SEQ ID NO: 8) |
| | Soluble TRAIL variant DR4-2 |
| | MAMMEVQGGPSLGQTCVLIVIFTVLLQSLCVAVTYVYFTNELKQMQDKYS |
| | KSGIACFLKEDDSYWDPNDEESMNSPCWQVKWQLRQLVRKMILRTSEETIS |
| | TVQEKQQNISPLVRERGPQRVAAHITGTRGRSNTLSSPNSKNEKALGRKINS |
| | WESSRRGHSFLSNLHLRNGELVIHEKGFYYIYSQTYFRFQEEIKENTKNDKQ |
| | MVQYIYKYTSYPDPILLMKSARNSCWSKDAEYGLYSIYQGGIFELKENDRIF |
| | VSVTNEHLIDMDHEASFFGAFLVG (SEQ ID NO: 9) |
| | Soluble TRAIL variant DR4-3 |
| | MAMMEVQGGPSLGQTCVLIVIFTVLLQSLCVAVTYVYFTNELKQMQDKYS |
| | KSGIACFLKEDDSYWDPNDEESMNSPCWQVKWQLRQLVRKMILRTSEETIS |
| | TVQEKQQNISPLVRERGPQRVAAHITGTRRRSNTLSSPNSKNEKALGIKINS |
| | WESSRRGHSFLSNLHLRNGELVIHEKGFYYIYSQTYFRFQEEIKENTKNDKQ |
| | MVQYIYKYTDYPDPILLMKSARNSCWSKDAEYGLYSIYQGGIFELKENDRI |
| | FVSVTNEHLIDMDHEASFFGAFLVG (SEQ ID NO: 10) |
| | Soluble TRAIL variant DR5-1 |
| | MAMMEVQGGPSLGQTCVLIVIFTVLLQSLCVAVTYVYFTNELKQMQDKYS |
| | KSGIACFLKEDDSYWDPNDEESMNSPCWQVKWQLRQLVRKMILRTSEETIS |
| | TVQEKQQNISPLVRERGPQRVAAHITGTRGRSNTLSSPNSKNEKALGRKINS |
| | WESSRSGHSFLSNLHLRNGELVIHEKGFYYIYSQTYFRFQEEIKENTKNDKQ |
| | MVQYIYKYTSYPDPILLMKSARNSCWSKDAEYGLYSIYQGGIFELKENDRIF |
| | VSVTNEHLIDMHHEASFFGAFLVG (SEQ ID NO: 11) |
| | Soluble TRAIL variant DR5-2 |
| | MAMMEVQGGPSLGQTCVLIVIFTVLLQSLCVAVTYVYFTNELKQMQDKYS |
| | KSGIACFLKEDDSYWDPNDEESMNSPCWQVKWQLRQLVRKMILRTSEETIS |
| | TVQEKQQNISPLVRERGPQRVAAHITGTRGRSNTLSSPNSKNEKALGRKINS |
| | WESSRSGHSFLSNLHLRNGELVIHEKGFYYIYSQTYFRFQERIKENTKNDKQ |
| | MVQYIYKYTSYPDPILLMKSARNSCWSKDAEYGLYSIYQGGIFELKENDRIF |
| | VSVTNEHLIDMHHEASFFGAFLVG (SEQ ID NO: 12) |
| Anti-PD- | VQLVESGGGLVQPGGSLRLSCAASGFTFSDSWIHWVRQAPGKGLEWVAWI |
| L1 scFv | SPYGGSTYYADSVKGRFTISADTSKNTAYLQMNSLRAEDTAVYYCARRHW |
| | PGGFDYWGQGTLVTVSSGGGGSGGGGSGGGGSIQMTQSPSSLSASVGDRV |
| | TITCRASQDVSTAVAWYQQKPGKAPKLLIYSASFLYSGVPSRFSGSGSGTDF |
| | TLTISSLQPEDFATYYCQQYLYHPATFGQGTKVEIK (SEQ ID NO: 13) |
| phenylalanine | MKTLSQAQSKTSSQQFSFTGNSSANVIIGNQKLTINDVARVARNGTLVSLTN |
| ammonia | NTDILQGIQASCDYINNAVESGEPIYGVTSGFGGMANVAISREQASELQTNL |
| lyase | VWFLKTGAGNKLPLADVRAAMLLRANSHMRGASGIRLELIKRMEIFLNAG |
| (PAL) | VTPYVYEFGSIGASGDLVPLSYITGSLIGLDPSFKVDFNGKEMDAPTALRQL |
| | NLSPLTLLPKEGLAMMNGTSVMTGIAANCVYDTQILTAIAMGVHALDIQAL |
| | NGTNQSFHPFIHNSKPHPGQLWAADQMISLLANSQLVRDELDGKHDYRDH |
| | ELIQDRYSLRCLPQYLGPIVDGISQIAKQIEIEINSVTDNPLIDVDNQASYHGG |
| | NFLGQYVGMGMDHLRYYIGLLAKHLDVQIALLASPEFSNGLPPSLLGNRER |
| | KVNMGLKGLQICGNSIMPLLTFYGNSIADRFPTHAEQFNQNINSQGYTSATL |
| | ARRSVDIFQNYVAIALMFGVQAVDLRTYKKTGHYDARACLSPATERLYSA |
| | VRHVVGQKPTSDRPYIWNDNEQGLDEHIARISADIAAGGVIVQAVQDILPCLH |
| | (SEQ ID NO: 14) |
| Y vb | MADKLPNIVILATGGTIAGSAATGTQTTGYKAGALGVDTLINAVPEVKKLA |
| Asparaginase | NVKGEQFSNMASENMTGDVVLKLSQRVNELLARDDVDGVVITHGTDTVE |
| | ESAYFLHLTVKSDKPVVFVAAMRPATAISADGPMNLLEAVRVAGDKQSRG |
| | RGVMVVLNDRIGSARYITKTNASTLDTFKANEEGYLGVIIGNRIYYQNRIDK |
| | LHTTRSVFDVRGLTSLPKVDILYGYQDDPEYLYDAAIQHGVKGIVYAGMG |
| | AGSVSVRGIAGMRKAMEKGVVVIRSTRTGNGIVPPDEELPGLVSDSLNPAH |
| | ARILLMLALTRTSDPKVIQEYFHTY (SEQ ID NO: 15) |
| Anti-α4β7 | Heavy chain variable region: |
| | QVQLVQSGAEVKKPGASVKVSCKGSGYTFTSYWMHWVRQAPGQRLEWIG |
| | EIDPSESNTNYNQKFKGRVTLTVDISASTAYMELSSLRSEDTAVYYCARGG |
| | YDGWDYAIDYWGQGTLVTVSS (SEQ ID NO: 16) |
| | Light chain variable region: |
| | DVVMTQSPLSLPVTPGEPASISCRSSQSLAKSYGNTYLSWYLQKPGQSPQLL |
| | IYGISNRFSGVPDRFSGSGSGTDFTLKISRVEAEDVGVYYCLQGTHQPYTFG |
| | QGTKVEIK (SEQ ID NO: 17) |
| Human | SPGQGTQSENSCTHFPGNLPNMLRDLRDAFSRVKTFFQMKDQLDNLLLKES |
| IL10 | LLEDFKGYLGCQALSEMIQFYLEEVMPQAENQDPDIKAHVNSLGENLKTLR |
| | LRLRRCHRFLPCENKSKAVEQVKNAFNKLQEKGIYKAMSEFDIFINYIEAY |
| | MTMKIRN (SEQ ID NO: 18) |
| Clotting | ANSFLEEMKKGHLERECMEETCSYEEAREVFEDSDKTNEFWNKYKDGDQ |
| Factor X | CETSPCQNQGKCKDGLGEYTCTCLEGFEGKNCELFTRKLCSLDNGDCDQF |
| | CHEEQNSVVCSCARGYTLADNGKACIPTGPYPCGKQTLERRKRSVAQATSS |
| | SGEAPDSITWKPYDAADLDPTENPFDLLDFNQTQPERGDNNLTRIVGGQEC |
| | KDGECPWQALLINEENEGFCGGTILSEFYILTAAHCLYQAKRFKVRVGDRN |
| | TEQEEGGEAVHEVEVVIKHNRFTKETYDFDIAVLRLKTPITFRMNVAPACLP |
| | ERDWAESTLMTQKTGIVSGFGRTHEKGRQSTRLKMLEVPYVDRNSCKLSS |
| | SFIITQNMFCAGYDTKQEDACQGDSGGPHVTRFKDTYFVTGIVSWGEGCAR |
| | KGKYGIYTKVTAFLKWIDRSMKTRGLPKAKSHAPEVITSSPLK (SEQ ID |
| | NO: 19) |

TABLE 4
Autoimmune diseases and antigens

| Disease | Antigen |
|---|---|
| acute rheumatic fever | cross reactive antibodies to cardiac muscle |
| alopecia areata | trychohyalin, keratin 16 |
| ANCA-associated vasculitis | neutrophil cytoplasmic antigen, proteinase 3, |
| | myeloperodixase, bacterial permiability increasing |
| | factor |
| autoimmune gastritis | H, K adenosine triphosphatase |
| autoimmune hemolytic anemia | Rh blood group antigens, I antigen |
| autoimmune hepatitis | nuclear protein, liver-kidney microsome type 1, liver |
| | cytosol type 1 |
| autoimmune myocarditis | cardiac myosin |
| autoimmune thyroiditis | thyroid peroxidase, thyroglobulin, thyroid- |
| | stimulating hormone receptor |
| autoimmune uveitis | retinal arrestin (S-antigen) |
| dermatomyositis | mi2 ATPase |
| diabetes (type 1) | pancreatic beta cell antigen, insulin, proinsulin, |
| | preproinsulin, glutamate decarboxylase (GAD65), |
| | insulinoma antigen-2 (IA-2) |
| Goodpasture's syndrome | noncollagenous domain of basement membrane |
| | collagen type IV |
| Graves' disease | thyroid stimulating hormone receptor, thyrotropin |
| | receptor |
| Guillain-Barre syndrome | neurofascin-186, gliomedin, nodal adhesion |
| | molecueles |
| hypoglycemia | insulin receptor |
| idiopathic thrombocytopenic purpura | platelet integrin GpIIb, GpIIIa |
| insulin resistant diabetes | insulin receptor |
| membranous nephritis | phospholipase A2 |
| mixed essential cryoglobulinemia | rheumatoid factor IgG complexes |
| multiple sclerosis | myelin basic protein, proteolipid protein, myelin |
| | oligodendrocyte glycoprotein (MOG) |
| myasthenia gravis | acetylcholine receptor |
| myasthenia gravis-MUSC | muscarinic receptor |
| pemphigus/pemphigoid | epidermal cadherin |
| pemphigus vulgaris, pemphigus | desmoglein 1, desmoglein 3 |
| foliaceus | |
| pernicious anemia | intrinsic factor (Gastric) |
| polymyositis | nuclear and nucleolar antigen |
| primary biliary cirrhosis | neutrophil nuclear antigen, mitochondrial |
| | multienzyme complex |
| psoriasis | PSO p27 |
| rheumatoid arthritis | rheumatoid factor IgG complexes, synovial joint |
| | antigen, citrullinated protein, carbamylated protein |
| scleroderma/systemic sclerosis | scl-86, nucleolar scleroderma antigen |
| Sjögren's syndrome | SS-B, Lupus La protein |
| systemic lupus erythematosus | DNA, histones, ribosomes, snRNP, scRNP |
| vitiligo | VIT-90, VIT-75, VIT-40 |
| Wegener's granulomatosis | neutrophil nuclear antigen |
| anti-phospholipid syndrome (APS) & | beta-2 glycoprotein 1 |
| catastrophic APS | |
| chemotherapy-induced peripheral | neuronal antigens |
| neuropathy | |
| thrombotic thrombocytopenic purpura | ADAMTS13 |
| atypical hemolytic uremic syndrome | complement factor H (CFH) |
| experimental autoimmune | myelin oligodendrocyte glycoprotein (MOG), myelin |
| encephalomyelitis (EAE), multiple | basic protein (MBP), proteolipid protein (PLP) |
| sclerosis (MS) | |
| neuromyelitis optica (NMO) | aquaporin 4 (AQP4) |
| Membranous glomerulonephritis | PLA2R |

### EXAMPLES

### Example 1

### Generation of an Enucleated Erythroid Cell with Increased Levels of an Endogenous Protein

A construct to specifically induce CD47 is produced. This construct uses dCpf1 to provide specific binding, though other polypeptides such as Cas9, a zinc finger protein, or a TALE protein could be substituted. dCpf1 is fused to transcriptional activators, and to an NLS to facilitate nuclear import. A guide RNA is also produced to provide target specificity.

More specifically, the construct is designed and made as follows. First, target-binding sites are chosen for dCpf1 upstream of the CD47 transcription start site. Four target sequences with 5′TTTN PAM sequences, suitable for use with a dLbCpf1 effector, are shown in the Table below. In this Example, all four target sequences are used in order to deliver four molecules of dCpf1 to the CD47 promoter.

TABLE
Exemplary CD47 target sequences.

| Location | | PAM | | SEQ |
|---|---|---|---|---|
| (GRCh37, | | (5′- | | ID |
| chr3) | Strand | TTTN) | Target Sequence 5′-3′ | NO: |
| 107810424 | + | TTTC | CTCCGGACGCGGCCGTCTAGC | 20 |
| 107810248 | + | TTTC | GCGCGGCGCTGGGCCCCACTG | 21 |
| 107810680 | − | TTTG | CGACAATGCTCGCTAGTCCCG | 22 |
| 107810302 | + | TTTC | ACTCCCACCCTCGCGCTTCAG | 23 |

A guide RNA specific for the target sequence is designed, and a vector encoding the guide RNA is assembled. Specifically, the guide crRNA is assembled in a multiplex array shown below. crRNA expression is driven by U6 promotor (dotted underlining). The four crRNA sequences (double underlining) are separated by Lachnospiraceae bacterium direct repeat stem loops (single underlining). An RNA polymerase termination symbol (dashed underlining) is downstream of the crRNAs and direct repeat stem loops. Alternative crRNAs can be designed, e.g., using the UCSC Genome browser (available on the world wide web at genome.ucsc.edu) or the FANTOM5/CAGE promoter atlas (available on the world wide web at fantom.gsc.riken.jp/5/).

(SEQ ID NO: 24)

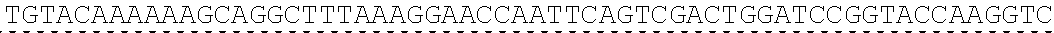

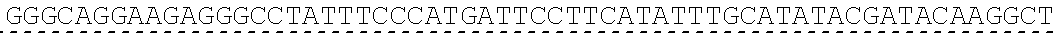

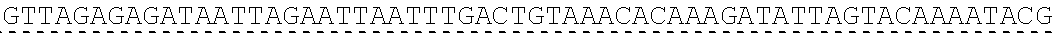

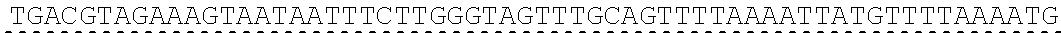

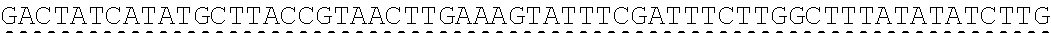

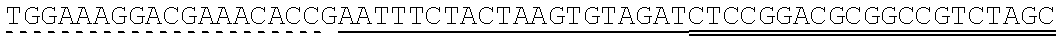

AATTTCTACTAAGTGTAGATGCGCGGCGCTGGGCCCCACTGAATTTCTACTAAGTGTAGA
TCGACAATGCTCGCTAGTCCCGAATTTCTACTAAGTGTAGATACTCCCACCCTCGCGCTT

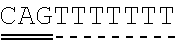

Next, the dCpf1-VPR protein construct is produced. This construct comprises, from N-terminus to C-terminus, the D832 nuclease-dead CRISPR protein dLbCpf1 (D832A) (amino acids 1-1228 of the sequence below), a nuclear localization sequence (NLS) (amino acids 1229-1244 of the sequence below), the transcriptional activator VP64 (amino acids 1245-1318 of the sequence below), the human NF-KB p65 activation domain (p65, amino acids 1319-1579 of the sequence below), and the Epstein-Barr-virus-derived R transactivator (Rta, amino acids 1586-1775 of the sequence below). See, e.g., Tak et al. (2017) Nature Methods 14: 1163-1166.

```
(SEQ ID NO: 25)
      1 MSKLEKFTNCYSLSKTLRFKAIPVGKTQENIDNKRLLVEDEKRAEDYKGV
    51 KKLLDRYYLSFINDVLHSIKLKNLNNYISLFRKKTRTEKENKELENLEIN
  101 LRKEIAKAFKGNEGYKSLFKKDIIETILPEFLDDKDEIALVNSFNGFTTA
  151 FTGFFDNRENMFSEEAKSTSIAFRCINENLTRYISNMDIFEKVDAIFDKH
  201 EVQEIKEKILNSDYDVEDFFEGEFFNFVLTQEGIDVYNAIIGGFVTESGE
  251 KIKGLNEYINLYNQKTKQKLPKFKPLYKQVLSDRESLSFYGEGYTSDEEV
  301 LEVFRNTLNKNSEIFSSIKKLEKLFKNFDEYSSAGIFVKNGPAISTISKD
  351 IFGEWNVIRDKWNAEYDDIHLKKKAVVTEKYEDDRRKSFKKIGSFSLEQL
  401 QEYADADLSVVEKLKEIIIQKVDEIYKVYGSSEKLFDADFVLEKSLKKND
  451 AVVAIMKDLLDSVKSFENYIKAFFGEGKETNRDESFYGDFVLAYDILLKV
  501 DHIYDAIRNYVTQKPYSKDKFKLYFQNPQFMGGWDKDKETDYRATILRYG
  551 SKYYLAIMDKKYAKCLQKIDKDDVNGNYEKINYKLLPGPNKMLPKVFFSK
  601 KWMAYYNPSEDIQKIYKNGTFKKGDMFNLNDCHKLIDFFKDSISRYPKWS
  651 NAYDFNFSETEKYKDIAGFYREVEEQGYKVSFESASKKEVDKLVEEGKLY
  701 MFQIYNKDFSDKSHGTPNLHTMYFKLLFDENNHGQIRLSGGAELFMRRAS
  751 LKKEELVVHPANSPIANKNPDNPKKTTTLSYDVYKDKRFSEDQYELHIPI
  801 AINKCPKNIFKINTEVRVLLKHDDNPYVIGIARGERNLLYIVVVDGKGNI
  851 VEQYSLNEIINNFNGIRIKTDYHSLLDKKEKERFEARQNWTSIENIKELK
  901 AGYISQVVHKICELVEKYDAVIALEDLNSGFKNSRVKVEKQVYQKFEKML
  951 IDKLNYMVDKKSNPCATGGALKGYQITNKFESFKSMSTQNGFIFYIPAWL
1001 TSKIDPSTGFVNLLKTKYTSIADSKKFISSFDRIMYVPEEDLFEFALDYK
1051 NFSRTDADYIKKWKLYSYGNRIRIFRNPKKNNVFDWEEVCLTSAYKELFN
1101 KYGINYQQGDIRALLCEQSDKAFYSSFMALMSLMLQMRNSITGRTDVDFL
1151 ISPVKNSDGIFYDSRNYEAQENAILPKNADANGAYNIARKVLWAIGQFKK
1201 AEDEKLDKVKIAISNKEWLEYAQTSVKHKRPAATKKAGQAKKKKEASGSG
1251 RADALDDFDLDMLGSDALDDFDLDMLGSDALDDFDLDMLGSDALDDFDLD
1301 MLINSRSSGSPKKKRKVGSQYLPDTDDRHRIEEKRKRTYETFKSIMKKSP
1351 FSGPTDPRPPPRRIAVPSRSSASVPKPAPQPYPFTSSLSTINYDEFPTMV
1401 FPSGQISQASALAPAPPQVLPQAPAPAPAPAMVSALAQAPAPVPVLAPGP
1451 PQAVAPPAPKPTQAGEGTLSEALLQLQFDDEDLGALLGNSTDPAVFTDLA
1501 SVDNSEFQQLLNQGIPVAPHTTEPMLMEYPEAITRLVTGAQRPPDPAPAP
1551 LGAPGLPNGLLSGDEDFSSIADMDFSALLGSGSGSRDSREGMFLPKPEAG
1601 SAISDVFEGREVCQPKRIRPFHPPGSPWANRPLPASLAPTPTGPVHEPVG
1651 SLTPAPVPQPLDPAPAVTPEASHLLEDPDEETSQAVKALREMADTVIPQK
1701 EEAAICGQMDLSHPPPRGHLDELTTTLESMTEDLNLDSPLTPELNEILDT
1751 FLNDECLLHAMHISTGLSIFDTSLF
```

Human pluripotent stem cells are obtained, cultured, and differentiated as described in Sugimura et al. (2017) Nature 545(7655):432-438.

Nucleic acid encoding the dCpf1-VPR and crRNA array is introduced into erythroid cell precursors using lentiviral transduction. Briefly, lentivirus is produced in 293T cells by transfecting the cells with lipofectamine. 20 μl (10 μg) pPACKH1 (SYSTEM BIOSCIENCES) plasmid mix+2 μg lenti construct+20 μl Plus reagent (LIFE TECHNOLOGIES) are combined in 400 μl Optimem and incubated 15 min at RT. 30 μl of LF2000 (LIFE TECHNOLOGIES) is diluted into 400 μl Optimem, added dropwise to DNA mix, and incubated for 15 min RT. DNA mix is added to cells. Cells are incubated for 6 hours and then the medium is changed to DMEM/10% FBS. The virus supernatant is collected 48 hours post-transfection by centrifugation at 1,500 rpm for 5 minutes. Target cells are then transduced. 5×10\hat{ }5 cultured cells are plated in 500 μL of medium containing 20 μg/mL polybrene in a 24-well plate. For each virus, cells are transduced in triplicate wells. Virus supernatant is added in another 500 μL of medium and the sample is mixed by pipetting. Infection is achieved by spinoculation, spinning the plate at 2000 rpm for 90 minutes at room temperature. After spinoculation, the cells are incubated at 37° C. overnight, and the next day 1 mL of fresh IMDM medium with appropriate cytokines is added.

Induction of CD47 expression is assayed. To detect CD47 mRNA levels, RT-PCR is performed using a Taqman primer/probe set (THERMO FISHER SCIENTIFIC Catalog# 4331182, Assay ID: Hs00179953_m1). To detect CD47 levels on the surface of the cells, FACS is performed using a CD47-FITC Monoclonal Antibody (THERMO FISHER SCIENTIFIC #11-0478-42). In some embodiments, CD47 expression in the transduced erythroid cells is at least 10%, 20%, 30%, 40%, 50%, 2-fold, 5-fold, or 10-fold higher than CD47 expression in an otherwise similar cell not transduced with the CRISPR component.

### Example 2

### Generation of an Enucleated Erythroid Cell Lacking an Endogenous Protein

A construct to specifically downregulate CD58 is produced. This construct uses a TALE protein to provide specific binding, though other polypeptides such as a zinc finger protein, Cas9, or Cpf1 could be substituted. The TALE protein is fused to the KRAP transcriptional repressor domain and to an NLS to facilitate nuclear import.

More specifically, the construct is designed and made as follows. First, target-binding sites are chosen for the TALE protein downstream of the CD58 transcription start site. A suitable target-binding site for CD58 transcriptional repression is 5′-cccggcccacagcgacccgt-3′ (SEQ ID NO: 30). Next, a TALE protein specific for the target-binding site is designed, e.g., as described in Cermak et al. (2011) Nucleic Acids Research 39(12):e82. The TALE protein is fused to an NLS and a KRAB transcriptional repressor domain. A suitable fusion protein construct is shown below, comprising from N-terminus to C-terminus, an N-terminal TALE domain (dotted underline), a C-terminal TALE domain (dashed underlining), a TALE repeat domain (single underline), an NLS (double underlining), and a KRAB repressor domain (wave underline). The TALE repeat domain contains repeat variable diresidues that confer specific binding to the target-binding site: diresidues HD binds to cytosine, diresidues NI binds to adenine, diresidues NG binds to thymine, and diresidues NH binds to guanine.

Full TALE-NLS-KRAB sequence to repress CD58:

(SEQ ID NO: 26)

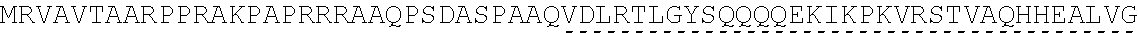

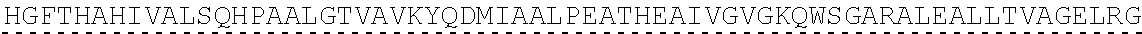

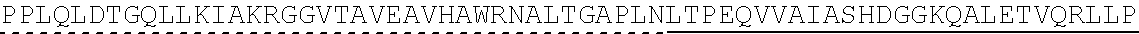

VLCQAHGLTPEQVVAIASHDGGKQALETVQRLLPVLCQAHGLTPEQVVAIASHDGGKQALETVQR
LLPVLCQAHGLTPEQVVAIASNHGGKQALETVQRLLPVLCQAHGLTPEQVVAIASNHGGKQALET
VQRLLPVLCQAHGLTPEQVVAIASHDGGKQALETVQRLLPVLCQAHGLTPEQVVAIASHDGGKQA
LETVQRLLPVLCQAHGLTPEQVVAIASHDGGKQALETVQRLLPVLCQAHGLTPEQVVAIASNIGG
KQALETVQRLLPVLCQAHGLTPEQVVAIASHDGGKQALETVQRLLPVLCQAHGLTPEQVVAIASN
IGGKQALETVQRLLPVLCQAHGLTPEQVVAIASNHGGKQALETVQRLLPVLCQAHGLTPEQVVAI
ASHDGGKQALETVQRLLPVLCQAHGLTPEQVVAIASNHGGKQALETVQRLLPVLCQAHGLTPEQV
VAIASNIGGKQALETVQRLLPVLCQAHGLTPEQVVAIASHDGGKQALETVQRLLPVLCQAHGLTP
EQVVAIASHDGGKQALETVQRLLPVLCQAHGLTPEQVVAIASHDGGKQALETVQRLLPVLCQAHG

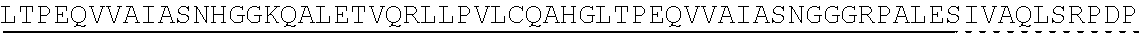

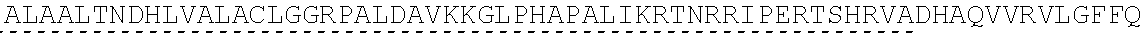

CHSHPAQAFDDAMTQFGMSRHGLLQLFRRVGVTELEARSGTLPPASQRWDRILQASGMKRAKPSP

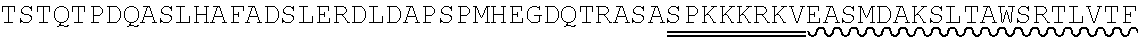

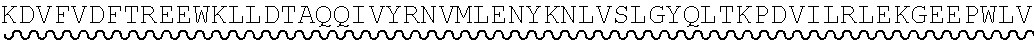

Human pluripotent stem cells are obtained, cultured, and differentiated as described in Sugimura et al., “Haematopoietic stem and progenitor cells from human pluripotent stem cells” Nature 2017 May 25; 545(7655):432-438. Nucleic acid encoding TALE-NLS-KRAB protein is introduced into erythroid cell precursors using lentiviral transduction as described in the previous example.

Repression of CD58 expression is assayed. To detect CD58 mRNA levels, RT-PCR is performed using a Taqman primer/probe set (Thermo Fisher Scientific Catalog# 4331182, Assay ID: Hs00156385_m1). To detect CD58 levels on the surface of the cells, FACS is performed using a CD58-FITC Monoclonal Antibody (Thermo Fisher Scientific #MA1-19575). In some embodiments, CD47 expression in the transduced erythroid cells is at least 10%, 20%, 30%, 40%, 50%, 2-fold, 5-fold, or 10-fold higher than CD47 expression in an otherwise similar cell not transduced with the TALE component.

### Example 3

### Generation of CD47 Knockout Erythroid Cells

To generate CD47−knockout human erythroid cells, the following experiment was performed.

Human CD34+ cells derived from mobilized peripheral blood cells from normal human donors were purchased frozen from AllCells Inc. The expansion/differentiation procedure comprised 3 stages. In the first stage, thawed CD34+ erythroid precursors were cultured in Iscove's MDM medium comprising recombinant human insulin, human transferrin, recombinant human recombinant human stem cell factor, and recombinant human interleukin 3. In the second stage, erythroid cells were cultured in Iscove's MDM medium supplemented with human serum albumin, recombinant human insulin, human transferrin, human recombinant stem cell factor, human recombinant erythropoietin, and L-glutamine. In the third stage, erythroid cells were cultured in Iscove's MDM medium supplemented with human transferrin, recombinant human insulin, human recombinant erythropoietin, and heparin. The cultures were maintained at 37° C. in 5% CO2 incubator.

Briefly, 2.5×10⁶ D1 cells (cells in the first day of the third stage) were resuspended in 1×P3 Primary Cell Nucleofector™ Solution containing 0.56 U RNasin® ribonuclease inhibitor (PROMEGA), 3.375 μg CleanCap® mRNA encoding Streptococcus pyogenes SF370 Cas9 (TRILINK BIOTECHNOLOGIES, Cat. No. L-7606) and either 6 μM of a mixture of CD47-specific synthetic guide RNA (sgRNA) including 2′O-methyl analogs and phosphorothioate internucleotide linkages (5′-U*U*G*CACUACUAAAGUCA-3′ (SEQ ID NO: 27); 5′-C*U*U*GUUUAGAGCUCAU-3′ (SEQ ID NO: 28); and 5′-C*U*U*GUUUAGAGCUCCAU-3′ (SEQ ID NO: 29); INVITROGEN) or no sgRNA (as control), and electroporated. Following electroporation, cells were incubated at 37° C. for 10 minutes, and transferred to 80 μl media, and cultured at 37° C.

The presence of CD47 on the surface of the erythroid cells was determined by staining the cells with fluorescein isothiocyanate (FITC)-conjugated anti-human CD47 antibody (BIOLEGEND) and performing fluorescence-activated cell sorting (FACS) after electroporation. Staining was performed on cells two days after electroporation (when the population still comprises primarily nucleated erythroid cells) and seven days after electroporation (when the population comprises primarily enucleated erythroid cells.)

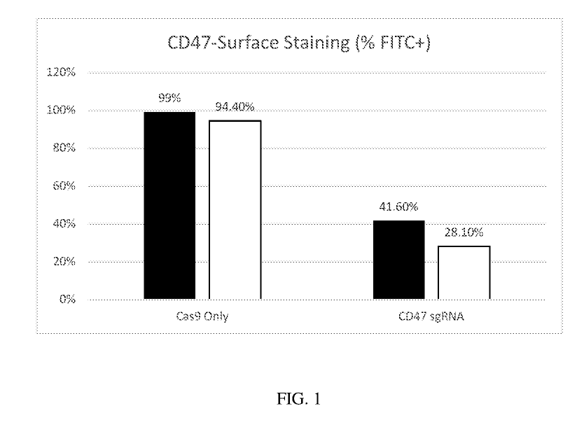

As shown in FIG. 1, knockout of CD47 was successful: two days after electroporation, only 41.60% of CD47 sgRNA-treated cells were positive for CD47, compared to 99% of Cas9-only controls being positive for CD47. Seven days after electroporation, only 28.1% of sgRNA-treated cells were positive for CD47, compared to 94.4% of Cas9-only controls being positive for CD47.

### Example 4

### CD47 Knockout Enucleated Erythroid Cells Exhibit Increased Attachment and Engulfment by Macrophages

To determine whether CD47 knockout enucleated erythroid cells are more readily engulfed by macrophages, the following experiment was performed.

Briefly, CD47 knockdown enucleated erythroid cells or control enucleated erythroid cell (electroporated with mRNA encoding Cas9 alone) were generated as described in Example 3. The enucleated erythroid cells were labeled with CellTrace™ Far Red (CTFR) (THERMOFISHER) according to manufacturer's instructions. 2×10⁵ lipopolysaccharide (LPS)-stimulated or 2 ×10⁵ non-stimulated adherently-cultured macrophages were trypsinized and admixed with either 2×10⁶ CD47 knockout enucleated erythroid cells or 2×10⁶ control enucleated erythroid cells, and incubated for approximately 16 hours at 37° C. After the incubation, supernatant media was removed, and cells were washed with 500 μL phosphate buffered saline (PBS), trypsinized using TrypLE (THERMOFISHER) for 15 minutes at 37° C., and detached from the culture plates using a scraper. Macrophages were stained with Pacific Blue-labelled anti-CD14 antibody (BIOLEGEND). Cells were analyzed using flow cytometry, whereby CD14-positive cells were indicative of all macrophages, and CD14-positive/CTFR-positive cells were indicative of macrophages that were attached to or had engulfed an erythroid cell.

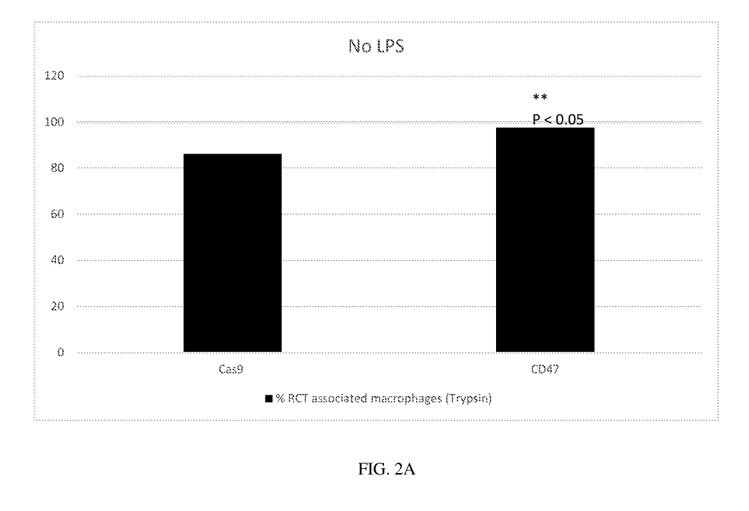

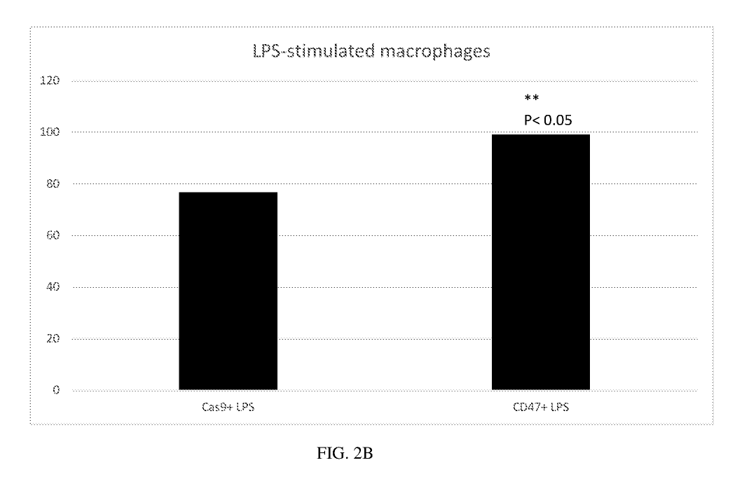

As shown in FIGS. 2A and 2B, both non-activated and LPS-stimulated macrophages exhibited increased adhesion and/or engulfment of CD47 knockout enucleated erythroid cells as compared to control enucleated erythroid cells. More specifically, FIG. 2A shows that 97.5% of cells in the CD47 knockout population adhered to or were engulfed by non-LPS stimulated macrophages, while only 86.1% of Cas9-only control cells were adhered to or were engulfed by the macrophages. This difference is statistically significant (p<0.05). FIG. 2B shows an even more striking difference, where 99.2% of cells in the CD47 knockout population adhered to or were engulfed by LPS-stimulated macrophages, while only 76.7% of Cas9-only control cells were adhered to or were engulfed by the macrophages. This difference is statistically significant (p<0.05). Both engulfment and attachment of the CD47− enucleated cells by macrophages were observed using an imaging flow cytometer (data not shown).

### Example 5

### Assessment of Immunogenicity and Tolerance Induction

### Tolerance Induction in Mice

In mice, tolerance can be induced by administering one ore mode doses (e.g., 3 sequential intravenous injections) of a population of enucleated erythroid cells described herein, e.g., CD47-negative cell comprising an antigenic protein, in this example ovalbumin (OVA). Naive mice are injected on days −7, −3 and −1 with either free OVA or enucleated erythroid cells that are CD47-negative and comprises OVA antigen. Mice are then immunized to OVA by two injections of the antigen mixed with poly I:C adjuvant (INVIVOGEN, San Diego, Calif.) to induce a strong immune response.

### Assessment of Antibody Titer

IgG levels in mouse serum are evaluated by standard ELISA. Briefly, serum is obtained at various time points from blood samples of mice that have been injected with the CD47-OVA+enucleated erythroid cells, and from mice that have been injected with free or recombinant OVA. A standard ELISA assay is used, with OVA as the antigen (1 μg/ml in 50 mM carbonate buffer, pH 9.7) adsorbed onto assay plates. Serum samples are serially diluted in the range of 1:50-1:200 for pre-treatment or no-treatment serum, and 1:400-1:500,000 for post-treatment serum and tested in duplicate. The binding of anti-OVA antibodies in serum to the adsorbed recombinant OVA is detected colorimetrically with a secondary anti-mouse immunoglobulin conjugated to horseradish peroxidase, followed by treatment with a chromogenic substrate.

Analysis of T cell Responses

Tolerance is induced to the antigenic protein OVA as described herein. Mice are euthanized 7 days after the 2nd administration of the immunization phase injection of OVA, and their spleens are collected. Spleen cell suspensions are obtained by straining the organs through a 70 micron cell strainer and after RBC lysis with a 0.8% ammonium chloride solution (STEM-CELL TECHNOLOGIES, Grenoble, France). All samples are incubated with anti-Fc receptor antibody (purified anti-CD16/32, OZYME, San Diego, Calif.) to prevent non-specific binding prior to incubations with antibodies for analysis. The following monoclonal antibodies (Abs) are used for spleen cell staining: PC5-anti-CD62L (MEL14) and PC7-anti-CD8, purchased from BIOLEGEND. OVA peptide-MHC tetramers (PE-Kb-SIINFEKL tetramers) are purchased from BECKMANN COULTER. OVA-specific T cells are identified by flow cytometry as cells that are double positive by staining with anti-CD8 and OVA peptide-MCH tetramers. Of this cell population, the percentage of OVA-specific CD8 T cells that are activated is determined by the fraction of cells that are positive by staining anti-CD62L antibody.

In vivo T cell Lysis Assay

Naive spleen cells are pulsed with 10 micrograms/ml of SIINFEKL (SEQ ID NO: 31) peptide (GENSCRIPT, Piscataway, N.J.) at 37C for 1 hour and then labeled with 0.4 microM CFSE (CFSE low). A control population of untreated splenocytes is labeled with 4 microM CFSE (CFSE high). CFSE low and CFSE high cells are combined in a ratio of 1:1 and 1E7 cells per mouse are injected by i.v. route to mice that have previously been tolerized to OVA antigen as described herein or to mice that have been immunized with OVA antigen as described herein. Sixteen hours later, spleen single-cell suspensions are prepared and analyzed using flow cytometry to determine the CFSE low/CFSE high cell ratio.

### Example 6

### Treatment of Diseases

1. Hemophilia

A subject suffering from hemophilia A is diagnosed. A composition of CD47-negative enucleated erythroid cells comprising exogenous FVIII is prepared as described herein. 1×10⁹ of the cells are administered intravenously to the subject. The clotting rate is assessed with a standard in vitro clotting time assay known in the art. Circulating antibodies against FVIII are detected in serum as described herein. The levels of circulating antibodies are assessed to track the effectiveness of immune tolerance induction. If the clotting cascade activity is insufficient to ensure healthy coagulation, recombinant or isolated FVIII are administered concurrently intravenously in order to reduce the symptoms of hemophilia A.

2. Atypical Hemolytic Uremic Syndrome

A subject suffering from atypical hemolytic uremic syndrome (aHUS) is diagnosed. A composition of CD47-negative enucleated erythroid cells comprising exogenous CFH is prepared as described herein. 1×10⁹ of the cells are administered intravenously to the subject. The symptomatic hemolysis rate is assessed with a standard urinary hemolysis assay known in the art. Circulating antibodies against CFH are detected in serum (e.g., using an immunoassay). The levels of circulating antibodies are assessed to track the effectiveness of immune tolerance induction. The subject is administered the treatment until the symptoms of the disease are seen to ameliorate using the assays described herein.

3. Multiple Sclerosis

An individual with multiple sclerosis (MS) receives a single infusion of 1×10⁹ CD47-negative enucleated erythroid cells expressing exogenous myelin basic protein (MBP), produced and formulated as described herein. At the day of study drug administration, the subject is monitored in a phase 1 insubject unit for 24 hours. Measurement of the primary outcome is performed at month 3 and additional safety follow-up is performed until month 6 with consecutive clinical, MRI, and general physical examinations as well as clinical and laboratory analyses to assess adverse events and monitor MS disease activity. The procedure is repeated until tolerance is induced such that the symptoms of MS are ameliorated in the individual. See, for example, Lutterotti et al. Sci Transl Med 5, 188ra75 (2013).

Frequency of different cell subsets is analyzed in whole blood (EDTA tubes) by flow cytometry with the following antibody panels: for immune cell subsets (granulocytes, eosinophils, monocytes, and B, T, NK, and NK T cells): anti-CD45 labelled with PE-Cy7 (EBIOSCIENCE), anti-CD16 labelled withAPC-Cy7 (BIOLEGEND), anti-CD19 labelled with fluorescein isothiocyanate (FITC) (BECTON DICKINSON (BD)), anti-CD14 labelled with V450 (BD), anti-CD3 labelled with peridinin chlorophyll protein (PerCP) (BD), and anti-CD56 labelled withphycoerythrin (PE) (EBIOSCIENCE); for T cell subsets including CD4+, FoxP3+ Tregs, regulatory CD8+CD57+ILT2+, and proinflammatory CD8+CD161high T cells: anti-CD3 labelled with PE-Cy7 (EBIOSCIENCE), anti-CD4 labelled with APC (EBIOSCIENCE), anti-CD8 labelled with Pacific Blue (PB) (DAKO-BIOZOL), anti-FoxP3 labelled with PE (MILTENYI), anti-CD25 labelled with APC (EBIOSCIENCE), anti-CD57 labelled with FITC (BD), anti-ILT2 labelled with PE (BECKMAN), and anti-CD161 labelled with APC (MILTENYI). The corresponding isotype controls are included in all stainings. Cells are analyzed with an LSR-II flow cytometer (BD) and FACSDiva Software (BD).

Peripheral blood mononuclear cells (PBMCs) are isolated by Ficoll density gradient centrifugation (PAA), and functional phenotype of T cells is evaluated by intracellular cytokine staining as follows: 5×10⁵ freshly isolated PBMCs are incubated overnight in 200 ml of X-VIVO 15 (LONZA) in a sterile FACS tube. The next day, cells are stimulated with phorbol 12-myristate 13-acetate (50 ng/ml, SIGMA) and ionomycin (1 mg/ml, SIGMA) in the presence of brefeldin A (10 mg/ml, eBioscience) for 5 hours. After washing with phosphate-buffered saline, cells are stained with LiveDead kit (AMCYAN, INVITROGEN), fixed, permeabilized, and stained with different antibodies: anti-IL-17 labelled with Alexa Fluor 647 (EBIOSCIENCE), anti-IL-4 labelled with PE-Cy7 (BIOLEGEND), anti-IFN-gamma labelled with FITC (BIOLEGEND), anti-IL-10 labelled with PE (BIOLEGEND), anti-CD3 labelled with PE DAKO CYTOMATION), anti-CD4 labelled with PB (DAKO CYTOMATION), and anti-CD8 labelled with PB (BIOLEGEND), or with the corresponding isotype controls.

The antigen-specific T cell responses toward the MBP used in the study are measured in freshly isolated PBMCs before the tolerization procedure and after 3 months. Antigen-specific T cell responses are analyzed by proliferation assays with thymidine incorporation. Briefly, isolated PBMCs are seeded in 96-well plates at 1.5×10\hat{ }5 PBMCs per well in X-VIVO 15 medium (LONZA) with 1 mM of MBP. Forty-eight wells are seeded per antigen, and six wells only with medium as negative control in each plate. TTx (5 mg/ml) (NOVARTIS BEHRING) is used as positive control. On day 7, plates are incubated for 15 hours with 1 mCi of [³H] thymidine (HARTMANN ANALYTIC). [³H]-thymidine-pulsed plates are analyzed with a scintillation counter (WALLAC 1450, PERKINELMER). The scintillation counts (CPM) of each well are measured. Wells showing CPM higher than the mean+3 SDs of the unstimulated wells will be considered as positive.

1. An erythroid cell lacking an endogenous protein chosen from CD47, CD58, PLSCR1 (Scramblase), or Bim, or having the endogenous protein present at a level less than about 50%, compared to an unmodified otherwise similar erythroid cell.

2. The erythroid cell of claim 1, which is an enucleated erythroid cell.

3. The erythroid cell of claim 2, which is a reticulocyte or a mature red blood cell.

4. The erythroid cell of claim 1, wherein the erythroid cell is substantially purified.

5. The erythroid cell of claim 1, wherein the erythroid cell was produced using an exogenous site-specific DNA binding protein.

6. The erythroid cell of claim 5, wherein the exogenous site-specific DNA binding protein is selected from a Cas9 polypeptide, ZF polypeptide, TALE polypeptide, or a viral vector component.

7. The erythroid cell of claim 1, wherein the erythroid cell was produced using a nucleic acid inhibitor chosen from siRNA or shRNA.

8. The erythroid cell of claim 1, wherein the endogenous protein is CD47.

9. The erythroid cell of claim 8, which further comprises an exogenous polypeptide.

10. The erythroid cell of claim 9, wherein the exogenous polypeptide is an antigen.

11. The erythroid cell of claim 10, wherein the antigen is an autoimmune antigen.

12. The erythroid cell of claim 10, wherein the antigen is intracellular.

13. The erythroid cell of claim 10, wherein the antigen is present at the surface of the erythroid cell.

14. The erythroid cell of claim 8, which substantially lacks endogenous CD47 or CD47-encoding RNA.

15. The erythroid cell of claim 8, which was made by a process comprising genetically modifying the DNA locus encoding CD47.

16. A population of cells comprising a plurality of erythroid cells of claim 1.

17. The population of cells of claim 16, which is at least 60%, 65%, 70%, 75%, 80%, 85%, 90%, 95%, 96%, 97%, 98%, 99%, or 100% enucleated.

18. A method of inducing immune tolerance, reducing immune activation, or treating an autoimmune disease in a subject, comprising administering an effective number of cells of claim 8 to a subject in need thereof, thereby inducing immune tolerance, reducing immune activation, or treating the autoimmune disease in the subject.


SEQUENCE LISTING (31 sequences)

SEQ ID NO: 1 - protein, 15 aa, Unknown
  Description of Unknown Cas9 motif sequence
IGLDIGTNSVGWAVI

SEQ ID NO: 2 - protein, 8 aa, Unknown
  Description of Unknown Cas9 motif sequence
IVIEMARE

SEQ ID NO: 3 - protein, 27 aa, Unknown
  Description of Unknown Cas9 motif sequence
DVDHIVPQSFLKDDSIDNKVLTRSDKN

SEQ ID NO: 4 - protein, 8 aa, Unknown
  Description of Unknown Cas9 motif sequence
HHAHDAYL

SEQ ID NO: 5 - protein, 205 aa, Unknown
  Description of Unknown 4-1BBL sequence
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 9 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRRGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 10 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGIKINSWESSRRGHSFLSNLHLRNGELVIHEKGFYYIYSQTYFRFQEEIKENTKNDKQMVQYIYKYTDYPDPILLMKSARNSCWSKDAEYGLYSIYQGGIFELKENDRIFVSVTNEHLIDMDHEASFFGAFLVG

SEQ ID NO: 11 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 12 - protein, 281 aa, Artificial Sequence
  Description of Artificial Sequence Synthetic polypeptide
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQERIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 13 - protein, 238 aa, Artificial Sequence
  Description of Artificial Sequence Synthetic polypeptide
VQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 14 - protein, 567 aa, Unknown
  Description of Unknown PAL sequence
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 15 - protein, 328 aa, Unknown
  Description of Unknown Y vb Asparaginase sequence
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 16 - protein, 121 aa, Artificial Sequence [sequence omitted]

SEQ ID NO: 17 - protein, 112 aa, Artificial Sequence [sequence omitted]

SEQ ID NO: 18 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 19 - protein, 448 aa, Unknown [sequence omitted]

SEQ ID NO: 20 - DNA, 25 bp, Homo sapiens
tttcctccggacgcggccgtctagc

SEQ ID NO: 21 - DNA, 25 bp, Homo sapiens
tttcgcgcggcgctgggccccactg

SEQ ID NO: 22 - DNA, 25 bp, Homo sapiens
tttgcgacaatgctcgctagtcccg

SEQ ID NO: 23 - DNA, 25 bp, Homo sapiens
tttcactcccaccctcgcgcttcag

SEQ ID NO: 24 - DNA, 490 bp, Artificial Sequence
  Description of Artificial Sequence Synthetic polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaatttctactaagtgtagatctccggacgcggccgtctagcaatttctactaagtgtagatgcgcggcgctgggccccactgaatttctactaagtgtagatcgacaatgctcgctagtcccgaatttctactaagtgtagatactcccaccctcgcgcttcagttttttt

SEQ ID NO: 25 - protein, 1775 aa, Artificial Sequence
  Description of Artificial Sequence Synthetic polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 26 - protein, 1099 aa, Artificial Sequence
  Description of Artificial Sequence Synthetic polypeptide
MRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNHGGKQALETVQRLLPVLCQAHGLTPEQVVAIASNHGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNHGGKQALETVQRLLPVLCQAHGLTPEQVVAIASHDGGKQALETVQRLLPVLCQAHGLTPEQVVAIASNHGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNHGGKQALETVQRLLPVLCQAHGLTPEQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASASPKKKRKVEASMDAKSLTAWSRTLVTFKDVFVDFTREEWKLLDTAQQIVYRNVMLENYKNLVSLGYQLTKPDVILRLEKGEEPWLV

SEQ ID NO: 27 - RNA, 17 nt, Artificial Sequence
  Description of Artificial Sequence Synthetic oligonucleotide
uugcacuacuaaaguca

SEQ ID NO: 28 - RNA, 16 nt, Artificial Sequence
  Description of Artificial Sequence Synthetic oligonucleotide
cuuguuuagagcucau

SEQ ID NO: 29 - RNA, 17 nt, Artificial Sequence
  Description of Artificial Sequence Synthetic oligonucleotide
cuuguuuagagcuccau

SEQ ID NO: 30 - DNA, 20 bp, Artificial Sequence
  source: /note=“Description of Artificial Sequence Synthetic oligonucleotide”
cccggcccacagcgacccgt

SEQ ID NO: 31 - protein, 8 aa, Gallus gallus
SIINFEKL